(19) United States
(12) Patent Application Publication
(10) Pub. No.: US 2011/0178154 A1
(43) Pub. Date: Jul. 21, 2011

(54) GENE EXPRESSION PROFILE THAT PREDICTS OVARIAN CANCER SUBJECT RESPONSE TO CHEMOTHERAPY

(76) Inventors: Michael J. Birrer, Mt. Airy, MD (US); Laurent L. Ozbun, Germantown, MD (US); Tomas A. Bonome, Washington, DC (US); Samuel Mok, Boston, MA (US)

(21) Appl. No.: 12/526,025

(22) PCT Filed: Feb. 6, 2008
(86) PCT No.: PCT/US2008/053225

§ 371 Date: Jan. 18, 2011

Related U.S. Application Data
(60) Provisional application No. 60/899,942, filed on Feb. 6, 2007.

(51) Int. Cl. A61K 31/713; C40B 30/00; C12Q 1/68; G01N 33/68; C40B 30/04; C40B 40/00; A61P 35/00

(52) U.S. Cl. 514/44 A; 506/7; 435/6.12; 435/6.18; 436/501; 506/9; 506/13

(57) ABSTRACT

A gene profiling signature is disclosed herein. The gene signature can predict whether a subject with ovarian cancer will be chemorefractory, chemoresistant or chemosensitive. Thus, methods are disclosed for determining whether a subject with ovarian cancer is sensitive to treatment with a chemotherapeutic agent. Methods are also provided for increasing sensitivity to the chemotherapeutic agent if the presence of differential expression indicates that the ovarian cancer has a decreased sensitivity to chemotherapeutic agent.

### CROSS REFERENCE TO RELATED APPLICATION

This application claims the benefit of U.S. Provisional Application No. 60/899,942, filed on Feb. 6, 2007, which is incorporated herein by reference in its entirety.

### FIELD OF THE DISCLOSURE

This disclosure relates to the field of cancer chemotherapy and in particular, to methods for predicting chemoresponsiveness in subjects with ovarian cancer and for identifying treatment modalities for subjects with ovarian cancer.

### BACKGROUND

Ovarian cancer is the fifth most common form of cancer in women in the United States, accounting for three percent of the total number of cancer cases and twenty-six percent of those occurring in the female genital tract. The American Cancer Society estimates that 15,310 deaths would be caused in women living in the United States in 2006. A large majority of women who die of ovarian cancer will have had serous carcinoma of the ovarian epithelium, a condition which occurs in sixty percent of all cases of ovarian cancer (Boring et al., Cancer J. Clin. 44: 7-26, 1994).

Women with ovarian cancer are typically asymptomatic until the cancer has metastasized. As a result, most women with ovarian cancer are not diagnosed until the cancer has progressed to an advanced and usually incurable stage (Boente et al., Curr. Probl. Cancer 20: 83-137, 1996). Survival rates are much better in women diagnosed with early-stage ovarian cancers, about ninety percent of these women are still alive five years after diagnosis.

Treatment of ovarian cancer typically involves a variety of treatment modalities. Generally, surgical intervention serves as the basis for treatment (Dennis S. Chi & William J Hoskins, Primary Surgical Management of Advanced Epithelial Ovarian Cancer, in Ovarian Cancer 241, Stephen C. Rubin & Gregory P. Sutton eds., 2d ed. 2001). Treatment of serous carcinoma often involves cytoreductive surgery (hysterectomy, bilateral salpingo-oophorectomy, omentectomy, and lymphadenectomy) followed by adjuvant chemotherapy with paclitaxel and either cisplatin or carboplatin (Eltabbakh, G. H. & Awtrey, C. S., Expert Op. Pharmacother. 2(10): 109-24, 2001).

Despite a clinical response rate of 80% to primary treatment with surgery and chemotherapy, most subjects experience tumor recurrence within two years of treatment. The overwhelming majority of subjects will eventually develop chemoresistance and die as a result of their cancer. Thus, a need exists to identify subjects that will develop chemoresistivity.

### SUMMARY OF THE DISCLOSURE

A gene profiling signature is disclosed herein that can be used to determine the chemotherapy response in subjects with ovarian cancer, such as papillary serous ovarian cancer. This gene signature can predict whether a subject will not respond to chemotherapy (chemorefractory), show an initial response but relapse within six months after a chemotherapy cycle is completed (chemoresistant), or will respond positively to chemotherapy (chemosensitive), for example, with a sensitivity of at least 71% and a specificity of at least 83% for a chemorefractory ovarian cancer and a sensitivity of at least 77% and a specificity of at least 83% for a chemoresistant ovarian cancer. Thus, methods of determining whether a subject with ovarian cancer will likely be sensitive to treatment with a chemotherapeutic agent are disclosed.

In one example, the method of determining if a subject is sensitive to treatment with a chemotherapeutic agent includes detecting expression of at least six chemotherapy sensitivity-related molecules in a sample obtained from the subject with ovarian cancer. The presence of differential expression of the at least six chemotherapy sensitivity-related molecules, for example relative to a reference value, indicates that the ovarian cancer has a decreased sensitivity to the chemotherapeutic agent. As such, the subject may not respond to the chemotherapeutic agent in a manner sufficient to treat the ovarian cancer. In an example, the at least six chemotherapy sensitivity-related molecules are represented by any of the molecules listed in Tables 1, such as ribonuclease L (2′,5′-oligoisoadenylate synthetase-dependent)(RNASEL)), REV3-like, catalytic subunit of DNA polymerase zeta (REV3L), DNA polymerase eta (POLH), collagen, type V, alpha 1(COL5A1), Dual-Specificity Phosphatase 1 (DUSP1), and collagen, type I, alpha 1 (COL1A1), and are indicative of a chemorefractory disease. In other examples, the at least six chemotherapy sensitivity-related molecules are selected from the list of chemotherapy sensitivity-related molecules shown in Table 5 and are indicative of chemoresistance.

In some examples, the methods include detecting expression of chemotherapy sensitivity-related molecules at either the nucleic acid level or protein level. In another example, the methods include determining whether a gene expression profile from the subject indicates chemoresponsiveness by using an array of molecules. In one example, the array includes oligonucleotides complementary to all chemotherapy sensitivity-related genes listed in Table 1 or all those listed in Table 5.

The disclosed gene expression signature has significant implications for the treatment of ovarian cancer. For example, the chemotherapy sensitivity-related molecules identified by the gene profile signature can serve as targets for specific molecular therapeutic molecules that can increase the sensitivity of ovarian cancer to standard chemotherapy. Thus, methods are disclosed for identifying an agent that alters the activity of a chemotherapy sensitivity-related molecule, such as RNASEL, POLH, COL5A1, DUSP1, REV3L, or COL1A1. Such identified agents can be used in ovarian cancer treatments.

In an example, a method of identifying an agent that alters an activity of a chemotherapy sensitivity-related molecule includes contacting an ovarian cancer cell with one or more test agents under conditions sufficient for the one or more test agents to alter the activity (such as the expression level) of at least six chemotherapy sensitivity-related molecules listed in Table 1, Table 5, or both Tables. The expression of the chemotherapy sensitivity-related molecules in the presence of the one or more test agents can be compared with expression in the absence of such agents. The presence of differential expression of the chemotherapy sensitivity-related molecules indicates that the test agent alters the activity of the one or more chemotherapy sensitivity-related molecules and thus may have therapeutic potential and can be selected for further analysis.

The disclosed methods can further include administering to the subject a therapeutically effective treatment to increase sensitivity to the chemotherapeutic agent if the presence of differential expression indicates that the ovarian cancer has a decreased sensitivity to a chemotherapeutic agent. In an example, the treatment includes administering a therapeutically effective amount of a composition, such as a specific binding agent that preferentially binds to one or more chemotherapy-sensitivity related molecules listed in Tables 1 and 5. For instance, the specific binding agent can be an inhibitor of one or more of the chemotherapy-sensitivity related molecules, such as a siRNA. Such inhibitors are useful for treatment of ovarian cancer.

Also disclosed are kits, including arrays, for determining chemoresponsive of an ovarian tumor. For example, an array can include one or more of the disclosed chemotherapy-sensitivity related molecules listed in Tables 1 and 5. Arrays can include other molecules, such as positive and negative controls.

The foregoing and other features of the disclosure will become more apparent from the following detailed description of several embodiments which proceeds with reference to the accompanying figures.

### BRIEF DESCRIPTION OF THE FIGURES

FIG. 1 is a graph illustrating the comparative fold change relative expression levels between microarray data and real-time quantitative RT-PCR data of selected genes from the refractory gene signature list provided in Table 1.

FIG. 2 is a graph illustrating the comparative fold change relative expression levels between microarray data and real-time quantitative RT-PCR data of selected genes from the resistant gene signature list provided in Table 5.

FIG. 3 is a graph illustrating that the A2780CP20 ovarian cancer cell line has increased sensitivity to cisplatin following pretreatment with POLH siRNAs.

FIG. 4 is a graph illustrating that the A2780CP20 ovarian cancer cell line has increased sensitivity to cisplatin following pretreatment with REV3L siRNAs.

FIG. 5 is a graph illustrating that the A2780CP20 ovarian cancer cell line has increased sensitivity to cisplatin following pretreatment with POLH and REV3L siRNAs.

FIG. 6 is a digital image illustrating the ability of POLH-5 siRNA to reduce or inhibit the expression of POLH 24 hours, 48 hours, 72 hours or 96 hours post-transfection with POLH-5 siRNA.

FIG. 7 is a bar graph illustrating the ability of combination POLH siRNA and cisplatin therapy to significantly reduce tumor weight.

### DETAILED DESCRIPTION OF SEVERAL EMBODIMENTS

### I. Introduction

Chemoresistance is a main contributor to the lethality of ovarian cancer. The inventors have identified a gene expression profile from ovarian carcinoma samples that can predict the response to chemotherapy with a sensitivity of at least 71% and a specificity of at least 83% for a chemorefractory ovarian cancer and a sensitivity of at least 77% and a specificity of at least 83% for a chemoresistant ovarian cancer in subjects that have been diagnosed with ovarian cancer, such as papillary serous ovarian cancer. For example, the disclosed gene profiling signature can predict if a subject will be refractory, resistant or sensitive to standard chemotherapy. This finding is important for it allows a subject's likely response to chemotherapy to be determined prior to receiving the treatment.

The disclosed gene signature also identifies genes and collections or sets of genes that serve as effective molecular markers for chemoresistance/chemorefraction in ovarian cancer, as well as such genes or gene sets that can provide clinically effective therapeutic targets for ovarian cancer. This has implications for the treatment of ovarian cancer. For example, methods are disclosed for increasing the sensitivity of a subject with ovarian cancer to a chemotherapeutic agent by targeting the chemotherapy sensitivity-related molecules identified by the gene profile signature. In an example, a therapeutically effective amount of a specific binding agent is administered to a subject. For example, the specific binding agent preferentially binds to one or more of the identified chemotherapy-sensitivity related molecules listed in Tables 1, 5, or both Tables. If the chemotherapy-sensitivity related molecule is up-regulated or overexpessed in a chemoresistant or chemorefractory tumor, a specific binding agent that inhibits such molecule can be administered. Alternatively, if the chemotherapy-sensitivity related molecule is downregulated in such tumor, a specific binding agent that activates this molecule (for example, expression or activity of the molecule) can be administered.

In a particular example, the specific binding agent preferentially binds to one or more molecules associated with a chemorefractory disease as listed in Table 1, such as agents that reduce or inhibit biological activity or expression of one or more of RNASEL, POLH, COL5A1, DUSP1, REV3L, or COL1A1. In another particular example, the specific binding agent binds to one or more molecules associated with chemoresistance, such as those listed in Table 5. In one example, the specific binding agent is an inhibitor, such as a siRNA, of one or more of the disclosed chemotherapy sensitivity-related molecules, such as those that are upregulated in subjects with a ovarian tumor resistant/refractory to chemotherapy.

### II. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

Administration: To provide or give a subject an agent, such as a chemotherapeutic agent, by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Amplifying a nucleic acid molecule: To increase the number of copies of a nucleic acid molecule, such as a gene or fragment of a gene, for example a region of a chemotherapy sensitivity-related gene. The resulting products are called amplification products.

An example of in vitro amplification is the polymerase chain reaction (PCR), in which a biological sample obtained from a subject (such as a sample containing ovarian cancer cells) is contacted with a pair of oligonucleotide primers, under conditions that allow for hybridization of the primers to a nucleic acid molecule in the sample. The primers are extended under suitable conditions, dissociated from the template, and then re-annealed, extended, and dissociated to amplify the number of copies of the nucleic acid molecule. Other examples of in vitro amplification techniques include quantitative real-time PCR, strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

A commonly used method for real-time quantitative polymerase chain reaction involves the use of a double stranded DNA dye (such as SYBR Green I dye). For example, as the amount of PCR product increases, more SYBR Green I dye binds to DNA, resulting in a steady increase in fluorescence. Another commonly used method is real-time quantitative TaqMan PCR (Applied Biosystems). This type of PCR has reduced the variability traditionally associated with quantitative PCR, thus allowing the routine and reliable quantification of PCR products to produce sensitive, accurate, and reproducible measurements of levels of gene expression. The 5′ nuclease assay provides a real-time method for detecting only specific amplification products. During amplification, annealing of the probe to its target sequence generates a substrate that is cleaved by the 5′ nuclease activity of Taq DNA polymerase when the enzyme extends from an upstream primer into the region of the probe. This dependence on polymerization ensures that cleavage of the probe occurs only if the target sequence is being amplified. The use of fluorogenic probes makes it possible to eliminate post-PCR processing for the analysis of probe degradation. The probe is an oligonucleotide with both a reporter fluorescent dye and a quencher dye attached. While the probe is intact, the proximity of the quencher greatly reduces the fluorescence emitted by the reporter dye by Förster resonance energy transfer (FRET) through space. Probe design and synthesis has been simplified by the finding that adequate quenching is observed for probes with the reporter at the 5′ end and the quencher at the 3′ end.

Antibody: A polypeptide ligand comprising at least a light chain or heavy chain immunoglobulin variable region which specifically recognizes and binds an epitope of an antigen, such as a COL1A1, COL5A1, DUSP1, POLH, RNASEL, or REV3L protein or a fragment thereof. Antibodies are composed of a heavy and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′2 fragments, single chain Fv proteins (“scFv”), and disulfide stabilized Fv proteins (“dsFv”). The term also includes recombinant forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, Immunology, 3rd Ed., W.H. Freeman & Co., New York, 1997.

Array: An arrangement of molecules, such as biological macromolecules (such as peptides or nucleic acid molecules) or biological samples (such as tissue sections), in addressable locations on or in a substrate. A “microarray” is an array that is miniaturized so as to require or be aided by microscopic examination for evaluation or analysis. Arrays are sometimes called DNA chips or biochips.

The array of molecules (“features”) makes it possible to carry out a very large number of analyses on a sample at one time. In certain example arrays, one or more molecules (such as an oligonucleotide probe) will occur on the array a plurality of times (such as twice), for instance to provide internal controls. The number of addressable locations on the array can vary, for example from at least one, to at least 6, to at least 10, at least 20, at least 30, at least 50, at least 75, at least 100, at least 150, at least 200, at least 300, at least 500, least 550, at least 600, at least 800, at least 1000, at least 10,000, or more. In particular examples, an array includes nucleic acid molecules, such as oligonucleotide sequences that are at least 15 nucleotides in length, such as about 15-40 nucleotides in length. In particular examples, an array includes oligonucleotide probes or primers which can be used to detect sensitive to chemotherapy-associated sequences, such as at least one of those listed in Tables 1 and 5, such as at least 6, at least 10, at least 20, at least 30, at least 50, at least 60, at least 80, at least 100, at least 110, at least 120 of the sequences listed in any of Tables 1 and 5. In an example, the array is a commercially available such as a U133 Plus 2.0 oligonucleotide array from AFFYMETRIX® (AFFYMETRIX®, Santa Clara, Calif.).

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Protein-based arrays include probe molecules that are or include proteins, or where the target molecules are or include proteins, and arrays including nucleic acids to which proteins are bound, or vice versa. In some examples, an array contains antibodies to chemotherapy sensitivity-related proteins, such as any combination of those listed in Tables 1 and 5, such as at least 1, at least 6, at least 10, at least 20, at least 30, at least 50, at least 60, at least 80, at least 100, at least 110, at least 120 of the sequences listed in any of Tables 1 and 5.

Binding or stable binding: An association between two substances or molecules, such as the hybridization of one nucleic acid molecule to another (or itself), the association of an antibody with a peptide, or the association of a protein with another protein or nucleic acid molecule. An oligonucleotide molecule binds or stably binds to a target nucleic acid molecule if a sufficient amount of the oligonucleotide molecule forms base pairs or is hybridized to its target nucleic acid molecule (such as those listed in Tables 1 and 5), to permit detection of that binding.

Binding can be detected by any procedure known to one skilled in the art, such as by physical or functional properties of the target:oligonucleotide complex. For example, binding can be detected functionally by determining whether binding has an observable effect upon a biosynthetic process such as expression of a gene, DNA replication, transcription, translation, and the like.

Physical methods of detecting the binding of complementary strands of nucleic acid molecules, include but are not limited to, such methods as DNase I or chemical footprinting, gel shift and affinity cleavage assays, Northern blotting, dot blotting and light absorption detection procedures. For example, one method involves observing a change in light absorption of a solution containing an oligonucleotide (or an analog) and a target nucleic acid at 220 to 300 nm as the temperature is slowly increased. If the oligonucleotide or analog has bound to its target, there is a sudden increase in absorption at a characteristic temperature as the oligonucleotide (or analog) and target disassociate from each other, or melt. In another example, the method involves detecting a signal, such as a detectable label, present on one or both nucleic acid molecules (or antibody or protein as appropriate).

The binding between an oligomer and its target nucleic acid is frequently characterized by the temperature (Tₘ) at which 50% of the oligomer is melted from its target. A higher (Tₘ) means a stronger or more stable complex relative to a complex with a lower (Tₘ).

Cancer: The “pathology” of cancer includes all phenomena that compromise the well-being of the subject. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc. “Metastatic disease” refers to cancer cells that have left the original tumor site and migrate to other parts of the body for example via the bloodstream or lymph system.

cDNA (complementary DNA): A piece of DNA lacking internal, non-coding segments (introns) and regulatory sequences which determine transcription. cDNA can be synthesized by reverse transcription from messenger RNA extracted from cells.

Chemorefractory or chemorefraction: A condition that does not respond to chemotherapy. For example, a tumor such as an ovarian tumor is chemorefractory if the tumor does not respond to the initial chemotherapy treatment, such as platinum-paclitaxel chemotherapy.

Chemoresistant or chemoresistance: A condition that is initially responsive to chemotherapy treatment, but relapses within six months of completing the initial treatment. For example, a tumor is chemoresistant if the tumor initially responds to chemotherapy treatment, but reappears within approximately six months of completing such treatment.

Chemosensitive: A condition that is responsive to the initial chemotherapy treatment and does not relapse following completion of that therapy. In one example, the condition does not relapse within about six months following completion of the therapy.

Chemotherapeutic agent or Chemotherapy: Any chemical agent with therapeutic usefulness in the treatment of diseases characterized by abnormal cell growth. Such diseases include tumors, neoplasms, and cancer as well as diseases characterized by hyperplastic growth such as psoriasis. In one embodiment, a chemotherapeutic agent is an agent of use in treating ovarian cancer, such as papillary serous ovarian cancer. In one example, a chemotherapeutic agent is a radioactive compound. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). Chemotherapeutic agents used for treating ovarian cancer include carboplatin, cisplatin, paclitaxel, docetaxel, doxorubicin, epirubicin, topotecan, irinotecan, gemcitabine, iazofurine, gemcitabine, etoposide, vinorelbine, tamoxifen, valspodar, cyclophosphamide, methotrexate, fluorouracil, mitoxantrone and vinorelbine. Combination chemotherapy is the administration of more than one agent to treat cancer.

Chemotherapy sensitivity-related (or associated) molecule: A molecule whose expression affects the ability of a subject to respond to chemotherapy. Such molecules include, for instance, nucleic acid sequences (such as DNA, cDNA, or mRNAs) and proteins. Specific genes include those listed in Tables 1 and 5, as well as fragments of the full-length genes, cDNAs, or mRNAs (and proteins encoded thereby) whose expression is altered (such as upregulated or downregulated) in response to ovarian cancer. Expression of chemotherapy sensitivity-related molecules can be used to detect chemorefraction and chemoresistance.

Examples of chemotherapy sensitivity-related molecules whose expression is upregulated or downregulated in ovarian cancers that are chemoresistant or chemorefractory include sequences related to collagens, apoptosis, cell survival and DNA repair genes, such as those listed in Tables 1 and 5. In an example, a chemotherapy sensitivity-related molecule is any molecule listed in Tables 1 and 5. Specific examples of chemotherapy sensitivity-related molecules that are indicative of chemorefraction are provided in Table 1 and include RNASEL, POLH, COL5A1, DUSP1, REV3L, or COL1A1. Examples of chemotherapy sensitivity-related molecules that are indicative of chemoresistance are listed in Table 5.

Chemotherapy sensitivity-related molecules can be involved in or influenced by cancer in different ways, including causative (in that a change in a chemotherapy sensitivity-related molecule leads to development of or progression of ovarian cancer that is chemoresistant or chemorefractory) or resultive (in that development of or progression of ovarian cancer that is chemoresistant or chemorefractory, causes or results in a change in the chemotherapy sensitivity-related molecule).

Collagen, type I, alpha 1 or COL1A1: Collagens are among the most abundant extracellular matrix proteins in vertebrate organisms. They maintain the structural integrity of tissues and mediate a wide variety of cell-matrix interactions. Type I collagen is a heterotrimer composed of two polypeptides encoded by the COL1A1 and COL1A2 genes. Although both transcriptional and posttranscriptional mechanisms are involved in regulation, the concordance between mRNA levels and type I collagen synthesis suggests that the predominant mode of control is transcriptional.

In particular examples, expression of COL1A1 is increased in ovarian cancer cells that are chemorefractory. The term COL1A1 includes any COL1A1 gene, cDNA, mRNA, or protein from any organism and that is COL1A1 and is expressed at elevated levels in a chemorefractory ovarian cancer cell relative to a non-chemorefractory ovarian cancer cell.

Nucleic acid and protein sequences for COL1A1 are publicly available. For example, GenBank Accession Nos.: NM_{—}000088, X54876 and BC036531 disclose COL1A1 nucleic acid sequences, and GenBank Accession Nos.: AAB59373, AAH59281 and AAA52052 disclose COL1A1 protein sequences, all of which are incorporated by reference as provided by GenBank on Feb. 1, 2007.

In one example, COL1A1 includes a full-length wild-type (or native) sequence, as well as COL1A1 allelic variants, fragments, homologs or fusion sequences that retain the ability to be increased during treatment of a chemorefractory ovarian cancer with chemotherapeutic agents and/or modulate sensitivity to such agents. In certain examples, COL1A1 has at least 80% sequence identity, for example at least 85%, 90%, 95%, or 98% sequence identity to COL1A1. In other examples, COL1A1 has a sequence that hybridizes to AFFYMETRIX® Probe ID No. 202310_s_at (UniGene ID No. Hs.172928, Locus Link ID No. 1277) and retains COL1A1 activity (such as the capability to be expressed during treatment of ovarian cancer with chemotherapeutic agents and/or modulate sensitivity to such agents).

Collagen, type V, alpha 1 or COL5A1: A type of collagen that is synthesized by fibroblasts and has been reported to play a role in fibril assembly. For example, COL5A1 can co-polymerize with type I collagen to form heterotypic fibrils. In particular examples, expression of COL5A1 is increased in ovarian cancer samples that are chemorefractory. The term COL5A1 includes any COL5A1 gene, cDNA, mRNA, or protein from any organism and that is COL5A1 and is expressed during chemorefraction.

Nucleic acid and protein sequences for COL5A1 are publicly available. For example, GenBank Accession Nos.: NM_{—}000093, BC008760 and AB009993 disclose COL5A1 nucleic acid sequences, and GenBank Accession Nos.: AAH08760, NP 604447 and BAD26732 disclose COL5A1 protein sequences, all of which are incorporated by reference as provided by GenBank on Feb. 1, 2007.

In one example, COL5A1 includes a full-length wild-type (or native) sequence, as well as COL5A1 allelic variants, fragments, homologs or fusion sequences that retain the ability to be increased during treatment of a chemorefractory ovarian cancer with chemotherapeutic agents and/or modulate sensitivity to such agents. In certain examples, COL5A1 has at least 80% sequence identity, for example at least 85%, 90%, 95%, or 98% sequence identity to COL5A1. In other examples, COL5A1 has a sequence that hybridizes to AFFYMETRIX® Probe ID No. 203325_s_at (UniGene ID No. Hs.210283, Locus Link ID No. 1289) and retains COL5A1 activity (such as the capability to be expressed during treatment of ovarian cancer with chemotherapeutic agents and/or modulate sensitivity to such agents).

Complementarity and percentage complementarity: Molecules with complementary nucleic acids form a stable duplex or triplex when the strands bind, (hybridize), to each other by forming Watson-Crick, Hoogsteen or reverse Hoogsteen base pairs. Stable binding occurs when an oligonucleotide molecule remains detectably bound to a target nucleic acid sequence under the required conditions.

Complementarity is the degree to which bases in one nucleic acid strand base pair with the bases in a second nucleic acid strand. Complementarity is conveniently described by percentage, that is, the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between an oligonucleotide molecule and a target nucleic acid sequence (such as a chemotherapy sensitivity-related molecule, for example any of the genes listed in Tables 1 and 5) to achieve detectable binding. When expressed or measured by percentage of base pairs formed, the percentage complementarity that fulfills this goal can range from as little as about 50% complementarity to full (100%) complementary. In general, sufficient complementarity is at least about 50%, for example at least about 75% complementarity, at least about 90% complementarity, at least about 95% complementarity, at least about 98% complementarity, or even at least about 100% complementarity.

A thorough treatment of the qualitative and quantitative considerations involved in establishing binding conditions that allow one skilled in the art to design appropriate oligonucleotides for use under the desired conditions is provided by Beltz et al. Methods Enzymol. 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Contacting: Placement in direct physical association, including both a solid and liquid form. Contacting can occur in vitro with isolated cells or tissue or in vivo by administering to a subject.

Determining expression of a gene product: Detection of a level of expression in either a qualitative or quantitative manner, for example by detecting nucleic acid or protein by routine methods known in the art.

Diagnosis: The process of identifying a disease by its signs, symptoms and results of various tests. The conclusion reached through that process is also called “a diagnosis.” Forms of testing commonly performed include blood tests, medical imaging, urinalysis, and biopsy.

DNA (deoxyribonucleic acid): A long chain polymer which includes the genetic material of most living organisms (some viruses have genes including ribonucleic acid, RNA). The repeating units in DNA polymers are four different nucleotides, each of which includes one of the four bases, adenine, guanine, cytosine and thymine bound to a deoxyribose sugar to which a phosphate group is attached. Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

Differential expression: A difference, such as an increase or decrease, in the conversion of the information encoded in a gene (such as a chemotherapy sensitivity-related molecule) into messenger RNA, the conversion of mRNA to a protein, or both. In some examples, the difference is relative to a control or reference value, such as an amount of gene expression that is expected in an ovarian cancer cell from a subject who does not have ovarian cancer or has a chemosensitive ovarian cancer. Detecting differential expression can include measuring a change in gene expression. For example, the genes listed in Table 1 are differentially expressed in ovarian cancers that are chemorefractory as compared to ovarian cancers that are chemosensitive.

Downregulated or inactivation: When used in reference to the expression of a nucleic acid molecule, such as a gene, refers to any process which results in a decrease in production of a gene product. A gene product can be RNA (such as mRNA, rRNA, tRNA, and structural RNA) or protein. Therefore, gene downregulation or deactivation includes processes that decrease transcription of a gene or translation of mRNA. For example, the genes listed in Table 1 with a negative t-value are downregulated relative to expression of the gene in a subject with a chemosensitive ovarian cancer.

Examples of processes that decrease transcription include those that facilitate degradation of a transcription initiation complex, those that decrease transcription initiation rate, those that decrease transcription elongation rate, those that decrease processivity of transcription and those that increase transcriptional repression. Gene downregulation can include reduction of expression above an existing level. Examples of processes that decrease translation include those that decrease translational initiation, those that decrease translational elongation and those that decrease mRNA stability.

Gene downregulation includes any detectable decrease in the production of a gene product. In certain examples, production of a gene product decreases by at least 2-fold, for example at least 3-fold or at least 4-fold, as compared to a control (such an amount of gene expression in a normal cell). In one example, a control is a relative amount of gene expression or protein expression in a biological sample taken from a subject who does not have ovarian cancer.

Dual-Specificity Phosphatase 1 or DUSP1: A phosphatase (otherwise known as mitogen-activated protein kinase [MAPK] phosphatase 1) which dephosphorylates and inactivates MAPKs. DUSP1 participates in immune-mediated inflammatory diseases and the treatment thereof.

In particular examples, expression of DUSP1 is increased in ovarian cancer samples that are chemorefractory. The term DUSP1 includes any DUSP1 gene, cDNA, mRNA, or protein from any organism and that is DUSP1 and is expressed during chemorefraction.

Nucleic acid and protein sequences for DUSP1 are publicly available. For example, GenBank Accession Nos.: NM_{—}004417, NM_{—}013642 and NM_{—}053769 disclose DUSP1 nucleic acid sequences, and GenBank Accession Nos.: P28563, P28562 and Q64623 disclose DUSP1 protein sequences, all of which are incorporated by reference as provided by GenBank on Feb. 1, 2007.

In one example, DUSP1 includes a full-length wild-type (or native) sequence, as well as DUSP1 allelic variants, fragments, homologs or fusion sequences that retain the ability to be expressed during treatment of a chemorefractory ovarian cancer with chemotherapeutic agents and/or modulate sensitivity to such agents. In certain examples, DUSP1 has at least 80% sequence identity, for example at least 85%, 90%, 95%, or 98% sequence identity to DUSP1. In other examples, DUSP1 has a sequence that hybridizes to AFFYMETRIX® Probe ID No. 201041_s_at (UniGene ID No. Hs.171695, Locus Link ID No. 1843) and retains DUSP1 activity (such as the capability to be expressed during treatment of a chemorefractory ovarian cancer with chemotherapeutic agents and/or modulate sensitivity to such agents).

Expression: The process by which the coded information of a gene is converted into an operational, non-operational, or structural part of a cell, such as the synthesis of a protein. Gene expression can be influenced by external signals. For instance, exposure of a cell to a hormone may stimulate expression of a hormone-induced gene. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

The expression of one nucleic acid molecule can be altered relative to a nucleic acid molecule, such as a normal (wild type) nucleic acid molecule. Alterations in gene expression, such as differential expression, include but are not limited to: (1) overexpression; (2) underexpression; or (3) suppression of expression. Alternations in the expression of a nucleic acid molecule can be associated with, and in fact cause, a change in expression of the corresponding protein.

Protein expression can also be altered in some manner to be different from the expression of the protein in a normal (wild type) situation. This includes but is not necessarily limited to: (1) a mutation in the protein such that one or more of the amino acid residues is different; (2) a short deletion or addition of one or a few (such as no more than 10-20) amino acid residues to the sequence of the protein; (3) a longer deletion or addition of amino acid residues (such as at least 20 residues), such that an entire protein domain or sub-domain is removed or added; (4) expression of an increased amount of the protein compared to a control or standard amount; (5) expression of a decreased amount of the protein compared to a control or standard amount; (6) alteration of the subcellular localization or targeting of the protein; (7) alteration of the temporally regulated expression of the protein (such that the protein is expressed when it normally would not be, or alternatively is not expressed when it normally would be); (8) alteration in stability of a protein through increased longevity in the time that the protein remains localized in a cell; and (9) alteration of the localized (such as organ or tissue specific or subcellular localization) expression of the protein (such that the protein is not expressed where it would normally be expressed or is expressed where it normally would not be expressed), each compared to a control or standard. Controls or standards for comparison to a sample, for the determination of differential expression, include samples believed to be normal (in that they are not altered for the desired characteristic, for example a sample from a subject who does not have cancer, such as ovarian cancer) as well as laboratory values, even though possibly arbitrarily set, keeping in mind that such values can vary from laboratory to laboratory.

Laboratory standards and values may be set based on a known or determined population value (e.g., a value representing expression of a gene for a particular parameter, such as ovarian cancer chemorefraction, chemoresistance, or chemosensitivity) and can be supplied in the format of a graph or table that permits comparison of measured, experimentally determined values.

Gene expression profile (or fingerprint): Differential or altered gene expression can be measured by changes in the detectable amount of gene expression (such as cDNA or mRNA) or by changes in the detectable amount of proteins expressed by those genes. A distinct or identifiable pattern of gene expression, for instance a pattern of high and low expression of a defined set of genes or gene-indicative nucleic acids such as ESTs; in some examples, as few as one or two genes provides a profile, but more genes can be used in a profile, for example at least 3, at least 4, at least 5, at least 6, at least 10, at least 20, at least 25, at least 30, at least 50, at least 80, at least 120 or more. A gene expression profile (also referred to as a fingerprint) can be linked to a tissue or cell type (such as ovarian cancer cell), to a particular stage of normal tissue growth or disease progression (such as advanced ovarian cancer), or to any other distinct or identifiable condition that influences gene expression in a predictable way (e.g., chemoresistance, chemorefraction, and chemosensitive). Gene expression profiles can include relative as well as absolute expression levels of specific genes, and can be viewed in the context of a test sample compared to a baseline or control sample profile (such as a sample from a subject who does not have ovarian cancer or has a chemosensitive ovarian cancer). In one example, a gene expression profile in a subject is read on an array (such as a nucleic acid or protein array). For example, a gene expression profile is performed using a commercially available array such as a Human Genome U133 2.0 Plus Microarray from AFFYMETRIX® (AFFYMETRIX®, Santa Clara, Calif.).

Hybridization: To form base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (Detects Sequences that Share at Least 90% Identity)

| | |
|---|---|
| Hybridization: | 5x SSC at 65° C. for 16 hours |
| Wash twice: | 2x SSC at room temperature (RT) for |
| | 15 minutes each |
| Wash twice: | 0.5x SSC at 65° C. for 20 minutes each |

High Stringency (Detects Sequences that Share at Least 80% Identity or Greater)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (Detects Sequences that Share Greater than 50% Identity)

| | |
|---|---|
| Hybridization: | 6x SSC at RT to 55° C. for 16-20 hours |
| Wash at least twice: | 2x-3x SSC at RT to 55° C. for 20-30 minutes each. |

Inhibitor: Any chemical compound, nucleic acid molecule, peptide such as an antibody, specific for a gene product that can reduce activity of a gene product or directly interfere with expression of a gene, such as those genes listed in Table 1 or 5 that are upregulated in ovarian cancers that are chemoresistant or chemorefractory. An inhibitor of the disclosure, for example, can inhibit the activity of a protein that is encoded by a gene either directly or indirectly. Direct inhibition can be accomplished, for example, by binding to a protein and thereby preventing the protein from binding an intended target, such as a receptor. Indirect inhibition can be accomplished, for example, by binding to a protein's intended target, such as a receptor or binding partner, thereby blocking or reducing activity of the protein. Furthermore, an inhibitor of the disclosure can inhibit a gene by reducing or inhibiting expression of the gene, inter alia by interfering with gene expression (transcription, processing, translation, post-translational modification), for example, by interfering with the gene's mRNA and blocking translation of the gene product or by post-translational modification of a gene product, or by causing changes in intracellular localization.

Isolated: An “isolated” biological component (such as a nucleic acid molecule, protein, or cell) has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins. For example, an isolated cell, is a serous papillary ovarian cancer cell that is substantially separated from other ovarian cell subtypes, such as endometrioid, clear cell or mucinous subtypes.

Label: An agent capable of detection, for example by ELISA, spectrophotometry, flow cytometry, or microscopy. For example, a label can be attached to a nucleic acid molecule or protein, thereby permitting detection of the nucleic acid molecule or protein. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Nucleic acid array: An arrangement of nucleic acids (such as DNA or RNA) in assigned locations on a matrix, such as that found in cDNA arrays, or oligonucleotide arrays.

Nucleic acid molecules representing genes: Any nucleic acid, for example DNA (intron or exon or both), cDNA, or RNA (such as mRNA), of any length suitable for use as a probe or other indicator molecule, and that is informative about the corresponding gene.

Nucleic acid molecules: A deoxyribonucleotide or ribonucleotide polymer including, without limitation, cDNA, mRNA, genomic DNA, and synthetic (such as chemically synthesized) DNA. The nucleic acid molecule can be double-stranded or single-stranded. Where single-stranded, the nucleic acid molecule can be the sense strand or the antisense strand. In addition, nucleic acid molecule can be circular or linear.

The disclosure includes isolated nucleic acid molecules that include specified lengths of a chemotherapy sensitivity-related molecule nucleotide sequence, for sequences for genes listed in Tables 1 and 5. Such molecules can include at least 10, at least 15, at least 20, at least 25, at least 30, at least 35, at least 40, at least 45 or at least 50 consecutive nucleotides of these sequences or more, and can be obtained from any region of a chemotherapy sensitivity-related molecule.

Nucleotide: Includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Oligonucleotide: A plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Particular oligonucleotides and oligonucleotide analogs can include linear sequences up to about 200 nucleotides in length, for example a sequence (such as DNA or RNA) that is at least 6 nucleotides, for example at least 8, at least 10, at least 15, at least 20, at least 21, at least 25, at least 30, at least 35, at least 40, at least 45, at least 50, at least 100 or even at least 200 nucleotides long, or from about 6 to about 50 nucleotides, for example about 10-25 nucleotides, such as 12, 15 or 20 nucleotides.

Oligonucleotide probe: A short sequence of nucleotides, such as at least 8, at least 10, at least 15, at least 20, at least 21, at least 25, or at least 30 nucleotides in length, used to detect the presence of a complementary sequence by molecular hybridization. In particular examples, oligonucleotide probes include a label that permits detection of oligonucleotide probe:target sequence hybridization complexes.

Ovarian cancer: A malignant ovarian neoplasm (an abnormal growth located on or in the ovaries). Cancer of the ovaries includes ovarian carcinoma, papillary serous cystadenocarcinoma, mucinous cystadenocarcinoma, endometrioid tumors, celioblastoma, clear cell carcinoma, unclassified carcinoma, granulosa-thecal cell tumors, Sertoli-Leydig cell tumors, dysgerminoma, and malignant teratoma. The most common type of ovarian cancer is papillary serous carcinoma.

Surgery is generally performed in treatment of ovarian cancer and is frequently necessary for diagnosis. The type of surgery depends upon how widespread the cancer is when diagnosed (the cancer stage), as well as the type and grade of cancer. The surgeon may remove one (unilateral oophorectomy) or both ovaries (bilateral oophorectomy), the fallopian tubes (salpingectomy), and the uterus (hysterectomy). For some very early tumors (stage 1, low grade or low-risk disease), only the involved ovary and fallopian tube will be removed (called a “unilateral salpingo-oophorectomy,” USO), especially in young females who wish to preserve their fertility. In advanced disease as much tumor as possible is removed (debulking surgery). In cases where this type of surgery is successful, the prognosis is improved compared to subjects where large tumour masses (more than 1 cm in diameter) are left behind.

Chemotherapy is often used after surgery to treat any residual disease. For example, systemic chemotherapy often includes a platinum derivative with a taxane and in some examples is used to treat advanced ovarian cancer. Chemotherapy is also often used to treat subjects who have a recurrence.

Polymerase (DNA directed) eta or POLH: A DNA polymerase involved in translesion DNA synthesis on DNA templates damaged by ultraviolet light (UV). For example, DNA polymerase eta has been reported to be responsible for the group variant of xeroderma pigmentosum.

In particular examples, expression of POLH is increased in ovarian cancer samples that are chemorefractory. The term POLH includes any POLH gene, cDNA, mRNA, or protein from any organism and that is POLH and is expressed during chemorefraction.

Nucleic acid and protein sequences for POLH are publicly available. For example, GenBank Accession Nos.: NM_{—}006502, NM_{—}030715 and BC128366 disclose POLH nucleic acid sequences, and GenBank Accession Nos.: AAI28367, AAH15742 and NP_{—}006493 disclose POLH protein sequences, all of which are incorporated by reference as provided by GenBank on Feb. 1, 2007.

In one example, POLH includes a full-length wild-type (or native) sequence, as well as POLH allelic variants, fragments, homologs or fusion sequences that retain the ability to be increased during treatment of chemorefractory ovarian cancer with chemotherapeutic agents and/or modulate sensitivity to such agents. In certain examples, POLH has at least 80% sequence identity, for example at least 85%, 90%, 95%, or 98% sequence identity to POLH. In other examples, POLH has a sequence that hybridizes to AFFYMETRIX® Probe ID No. 233852_at (UniGene ID No. Hs.439153, Locus Link ID No. 5429) and retains POLH activity (such as the capability to be expressed during treatment of chemorefractory ovarian cancer with chemotherapeutic agents and/or modulate sensitivity to such agents).

Predisposition: Refers to an effect of a factor or factors that render a subject susceptible to a condition, disease, or disorder, such as cancer. In the context of this disclosure, the factor(s) that render the subject susceptible to the condition are genetic and/or epigenetic factors. In some instances testing is able to identify a subject predisposed to developing a condition, disease, or disorder, such as being resistant to chemotherapy for treating ovarian cancer.

Primers: Short nucleic acid molecules, for instance DNA oligonucleotides 10 -100 nucleotides in length, such as about 15, 20, 25, 30 or 50 nucleotides or more in length. Primers can be annealed to a complementary target DNA strand (e.g., such as to those listed in Tables 1 and 5) by nucleic acid hybridization to form a hybrid between the primer and the target DNA strand. Primer pairs can be used for amplification of a nucleic acid sequence, such as by PCR or other nucleic acid amplification methods known in the art.

Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990). PCR primer pairs can be derived from a known sequence, for example, by using computer programs intended for that purpose such as Primer (Version 0.5, © 1991, Whitehead Institute for Biomedical Research, Cambridge, Mass.). One of ordinary skill in the art will appreciate that the specificity of a particular primer increases with its length. Thus, for example, a primer including 30 consecutive nucleotides of a chemotherapy sensitivity-related nucleotide molecule will anneal to a target sequence, such as another homolog of the designated chemotherapy sensitivity-related protein, with a higher specificity than a corresponding primer of only 15 nucleotides. Thus, in order to obtain greater specificity, primers can be selected that include at least 20, at least 25, at least 30, at least 35, at least 40, at least 45, at least 50 or more consecutive nucleotides of a chemotherapy sensitivity-related nucleotide sequence.

Purified: The term “purified” does not require absolute purity; rather, it is intended as a relative term. Thus, for example, a purified protein preparation is one in which the protein referred to is more pure than the protein in its natural environment within a cell. For example, a preparation of a protein is purified such that the protein represents at least 50% of the total protein content of the preparation. Similarly, a purified oligonucleotide preparation is one in which the oligonucleotide is more pure than in an environment including a complex mixture of oligonucleotides.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

REV3-like, catalytic subunit of DNA polymerase zeta or REV3L: A product of the REV3 gene and reported to be involved in UV-induced mutagenesis. In particular examples, expression of REV3L is increased in ovarian cancer samples that are chemorefractory. The term REV3L includes any REV3L gene, cDNA, mRNA, or protein from any organism and that is REV3L and is expressed during chemorefraction.

Nucleic acid and protein sequences for REV3L are publicly available. For example, GenBank Accession Nos.: NM_{—}002912 and AY684169 disclose REV3L nucleic acid sequences, and GenBank Accession Nos.: CAI20998, CAI20997 and CAI20509 disclose REV3L protein sequences, all of which are incorporated by reference as provided by GenBank on Feb. 1, 2007.

In one example, REV3L includes a full-length wild-type (or native) sequence, as well as REV3L allelic variants, fragments, homologs or fusion sequences that retain the ability to be increased during treatment of a chemorefractory ovarian cancer with chemotherapeutic agents and/or modulate sensitivity to such agents. In certain examples, REV3L has at least 80% sequence identity, for example at least 85%, 90%, 95%, or 98% sequence identity to REV3L. In other examples, REV3L has a sequence that hybridizes to AFFYMETRIX® Probe ID No. 208070_{—}2_at (UniGene ID No. Hs.232021, Locus Link ID No. 5980) and retains REV3L activity (such as the capability to be expressed during treatment of a chemorefractory ovarian cancer with chemotherapeutic agents and/or modulate sensitivity to such agents).

Ribonuclease L (2′,5′-oligoisoadenylate synthetase-dependent) or RNASEL: An enzyme that has been implicated in the molecular mechanisms of interferon action and the fundamental control of RNA stability in mammalian cells.

In particular examples, expression of RNASEL is increased in ovarian cancer samples that are chemorefractory. The term RNASEL includes any RNASEL gene, cDNA, mRNA, or protein from any organism and that is RNASEL and is expressed during chemorefraction.

Nucleic acid and protein sequences for RNASEL are publicly available. For example, GenBank Accession Nos.: NM_{—}021133, NM_{—}011882 and NM_{—}182673 disclose RNASEL nucleic acid sequences, and GenBank Accession Nos.: AAP22025, AAH90934 and NP_{—}066956 disclose RNASEL protein sequences, all of which are incorporated by reference as provided by GenBank on Feb. 1, 2007.

In one example, RNASEL includes a full-length wild-type (or native) sequence, as well as RNASEL allelic variants, fragments, homologs or fusion sequences that retain the ability to be increased during treatment of chemorefractory ovarian cancer with chemotherapeutic agents and/or modulate sensitivity to such agents. In certain examples, RNASEL has at least 80% sequence identity, for example at least 85%, 90%, 95%, or 98% sequence identity to RNASEL. In other examples, RNASEL has a sequence that hybridizes to AFFYMETRIX® Probe ID No. 229285_at (UniGene ID No. Hs.518545, Locus Link ID No. 6041) and retains RNASEL activity (such as the capability to be expressed during treatment of chemorefractory ovarian cancer with chemotherapeutic agents and/or modulate sensitivity to such agents).

Sample (or biological sample): A biological specimen containing genomic DNA, RNA (including mRNA), protein, or combinations thereof, obtained from a subject. Examples include, but are not limited to, peripheral blood, urine, saliva, tissue biopsy, surgical specimen, amniocentesis samples and autopsy material. In one example, a sample includes a microdissected advanced stage, high-grade papillary serous ovarian cancer tissue biopsy.

Sensitivity: A measurement of activity, such as biological activity, of a molecule or a collection molecules in a given condition. In an example, sensitivity refers to the activity of any chemotherapeutic sensitivity-related molecule listed in Tables 1 and 5 in the presence of a chemotherapeutic agent. In other examples, sensitivity refers to the activity of an agent (such as a chemotherapeutic agent) on the growth, development or progression of a disease, such as ovarian cancer. For example, a decreased sensitivity refers to a state in which a tumor is less responsive to a given chemotherapeutic agent as compared to a tumor that is responsive to the treatment.

In certain examples, sensitivity or responsiveness can be assessed using any endpoint indicating a benefit to the subject, including, without limitation, (1) inhibition, to some extent, of tumor growth, including slowing down and complete growth arrest; (2) reduction in the number of tumor cells; (3) reduction in tumor size; (4) inhibition (such as reduction, slowing down or complete stopping) of tumor cell infiltration into adjacent peripheral organs and/or tissues; (5) inhibition (such as reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for

Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

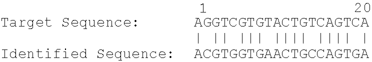

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. AppL Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a protein listed in Tables 1 and 5.

When aligning short peptides (fewer than around 30 amino acids), the alignment is be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, 99% sequence identity to a protein listed in Tables 1 and 5. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 60%, 70%, 80%, 90%, 95%, 98%, or 99% sequence identity to a nucleic acid listed in Tables 1 and 5 determined by this method. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Short interfering RNA (siRNA): A double stranded nucleic acid molecule capable of RNA interference or “RNAi.” (See, for example, Bass Nature 411: 428-429, 2001; Elbashir et al., Nature 411: 494-498, 2001; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914.) As used herein, siRNA molecules need not be limited to those molecules containing only RNA, but further encompasses chemically modified nucleotides and non-nucleotides having RNAi capacity or activity. In an example, an siRNA molecule is one that reduces or interferes with the biological activity of one or more chemotherapy sensitivity-related molecules disclosed in Tables 1 and 5, such as COL1A1, COL5A1, DUSP1, POLH, RNASEL or REV3L.

Subject: Living multi-cellular vertebrate organisms, a category that includes human and non-human mammals, such as veterinary subjects.

Target sequence: A sequence of nucleotides located in a particular region in the human genome that corresponds to a desired sequence, such as a chemotherapy sensitivity-related sequence. The target can be for instance a coding sequence; it can also be the non-coding strand that corresponds to a coding sequence. Examples of target sequences include those sequences associated with chemotherapy sensitivity, such as any of those listed in Tables 1 and 5.

Test agent: Any substance, including, but not limited to, a protein (such as an antibody), nucleic acid molecule (such as a siRNA), organic compound, inorganic compound, or other molecule of interest. In particular examples, a test agent can permeate a cell membrane (alone or in the presence of a carrier).

Therapeutically effective amount: An amount of a pharmaceutical preparation that alone, or together with a pharmaceutically acceptable carrier or one or more additional therapeutic agents, induces the desired response. A therapeutic agent, such as a chemotherapeutic agent, is administered in therapeutically effective amounts.

Effective amounts a therapeutic agent can be determined in many different ways, such as assaying for a reduction in tumor size or improvement of physiological condition of a subject having cancer, such as ovarian cancer. Effective amounts also can be determined through various in vitro, in vivo or in situ assays.

Therapeutic agents can be administered in a single dose, or in several doses, for example daily, during a course of treatment. However, the effective amount of can be dependent on the source applied, the subject being treated, the severity and type of the condition being treated, and the manner of administration.

In one example, it is an amount sufficient to partially or completely alleviate chemoresistance in the subject with ovarian cancer. Treatment can involve only slowing the progression to chemoresistance (for example resistance occurs after 6 months, such as 30 months from the initial chemotherapy treatment), but can also include halting or reversing chemoresistance/chemorefraction permanently. For example, a pharmaceutical preparation can decrease chemoresistance by at least 20%, at least 50%, at least 70%, at least 90%, at least 98%, or even at least 100%, as compared to chemoresistance observed in the absence of the pharmaceutical preparation. In other examples, a pharmaceutical preparation can render a chemorefractory tumor, chemosensitive.

Tissue: A plurality of functionally related cells. A tissue can be a suspension, a semi-solid, or solid. Tissue includes cells collected from a subject such as the ovaries or a portion thereof.

Treating a disease: “Treatment” refers to a therapeutic intervention that ameliorates a sign or symptom of a disease or pathological condition, such as a sign or symptom of ovarian cancer. Treatment can also induce remission or cure of a condition, such as ovarian cancer. In particular examples, treatment includes preventing a disease, for example by inhibiting the full development of a disease. Prevention of a disease does not require a total absence of disease. For example, a decrease of at least 50% can be sufficient.

Tumor: All neoplastic cell growth and proliferation, whether malignant or benign, and all pre-cancerous and cancerous cells and tissues.

Under conditions sufficient for: A phrase that is used to describe any environment that permits the desired activity. In one example, includes administering a test agent to an ovarian cancer cell or a subject sufficient to allow the desired activity. In particular examples, the desired activity is altering the activity (such as the expression) of a chemotherapy sensitivity-related molecule.

Upregulated or activation: When used in reference to the expression of a nucleic acid molecule, such as a gene, refers to any process which results in an increase in production of a gene product. A gene product can be RNA (such as mRNA, rRNA, tRNA, and structural RNA) or protein. Therefore, gene upregulation or activation includes processes that increase transcription of a gene or translation of mRNA. For example, the genes with a positive t-value in Table 1 are upregulated relative to expression of the gene in a subject with a chemosensitive ovarian cancer.

Examples of processes that increase transcription include those that facilitate formation of a transcription initiation complex, those that increase transcription initiation rate, those that increase transcription elongation rate, those that increase processivity of transcription and those that relieve transcriptional repression (for example by blocking the binding of a transcriptional repressor). Gene upregulation can include inhibition of repression as well as stimulation of expression above an existing level. Examples of processes that increase translation include those that increase translational initiation, those that increase translational elongation and those that increase mRNA stability.

Gene upregulation includes any detectable increase in the production of a gene product. In certain examples, production of a gene product increases by at least 2-fold, for example at least 3-fold or at least 4-fold, as compared to a control (such an amount of gene expression in a normal cell or in an ovarian cancer cell that is chemosensitive). In one example, a control is a relative amount of gene expression in a biological sample, such as in an ovarian tissue biopsy obtained from a subject that does not have ovarian cancer or has an overian cancer that is chemosensitive.

### Gene Expression Profile

Disclosed herein is a gene expression profile that can be used to determine the chemotherapeutic response in subjects with ovarian cancer, such as papillary serous ovarian cancer. This gene signature can be used to determine an ovarian cancer's sensitivity to a chemotherapeutic treatment, for example, to predict whether a subject will not respond to chemotherapy (referred to as chemorefactory), show an initial response but relapse (such as within six months) after completing the chemotherapy cycle (referred to as chemoresistant), or will respond positively to chemotherapy (referred to as chemosensitive). In some examples, the gene profile can predict with a sensitivity of at least 70% and a specificity of at least 80% for a chemorefractory ovarian cancer, such as a sensitivity of at least 75%, at least 80%, at least 85%, at least 90%, and at least 95% (for example, 71%, 72%, 73%, 74%, 75%, 76%, 77%, 78%, 79%, 80%, 83%, 86%, 90%, 91%, 92%, 93%, 94%, 95%, 96%, 97%, 98%, 99% or 100%) and a specificity of at least of at least 80%, at least 85%, at least 90%, and at least 95% (for example, 81%, 82%, 83%, 84%, 85%, 86%, 90%, 91%, 92%, 93%, 94%, 95%, 96%, 97%, 98%, 99% or 100%). In other examples, the gene profile can predict with a sensitivity of at least 70% and a specificity of at least 80% for a chemoresistant ovarian cancer, such as a sensitivity of at least 75%, at least 80%, at least 85%, at least 90%, and at least 95% (for example, 71%, 72%, 73%, 74%, 75%, 76%, 77%, 78%, 79%, 80%, 83%, 86%, 90%, 91%, 92%, 93%, 94%, 95%, 96%, 97%, 98%, 99% or 100%) and a specificity of at least 80%, at least 85%, at least 90%, and at least 95% (for example, 81%, 82%, 83%, 84%, 85%, 86%, 90%, 91%, 92%, 93%, 94%, 95%, 96%, 97%, 98%, 99% or 100%).

In an example, the gene expression profile includes at least six of the chemotherapy sensitivity-related molecules listed in Table 1 and/or Table 5, such as at least 10, at least 20, at least 30, at least 40, at least 50, at least 60, at least 70, at least 80, at least 90, at least 100, at least 110, at least 120, or at least 130 molecules (for example, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135 or 136 of those listed).

In a particular example, the gene expression profile includes at least 6, at least 10, at least 20, at least 30, at least 40, at least 50, at least 60, at least 70, at least 80, at least 90, or at least 100 molecules (for example, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or 105) of the molecules indicative of chemorefraction listed in Table 1. In a particular example, the at least six molecules that are indicative of chemorefraction include RNASEL, POLH, COL5A1, DUSP1, REV3L, and COL1A1.

In other particular examples, the gene expression profile includes at least 6, at least 10, at least 20, or at least 30 molecules (for example, 6, 7, 8, 9, 10, 11, 12, 13, 14, 15, 16, 17, 18, 19, 20, 25, 30, or 31) that are indicative of chemoresistance and is represented by any of the molecules listed in Table 5. For example, the profile can include thirty-one chemotherapy sensitivity-related molecules listed in Table 5.

### Chemotherapy Sensitivity-Related Molecules

Chemotherapy sensitivity-related molecules can include nucleic acid sequences (such as DNA, cDNA, or mRNAs) and proteins. In a specific example, detecting expression of the chemotherapy sensitivity-related molecules includes detecting mRNA expression of the disclosed chemotherapy sensitivity-related molecules. In another example, detecting expression of the chemotherapy sensitivity-related molecules includes detecting protein expression of the disclosed chemotherapy sensitivity-related molecules.

### Altered Chemotherapy Sensitivity-Related Molecule Expression

In an example, an alteration in the expression or biological activity of one or more of the disclosed chemotherapy sensitivity-related molecules includes an increase or decrease in production of a gene product, such as RNA or protein. For example, an alteration can include processes that downregulate or decrease transcription of a gene or translation of mRNA. Gene downregulation includes any dectable decrease in the production of a gene product. In certain examples, production/expression of a gene product decreases by at least 2-fold, for example at least 3-fold or at least 4-fold, as compared to a control or reference value (such an amount or range of amounts of gene expression expected in a normal ovarian cell or an ovarian cancern that is chemosensitive). For example, genes listed in Table 1 with a negative t-value, such as LOC11508, FAIM2, SLC5A1, Cl8orf30, MGC50559, LOC400752, PAIP2, CCNL1, SLC5A1 and CTSE, are downregulated in ovarian cancers that are chemorefractory relative to ovarian cancers that are chemosensitive.

In another example, an alteration can include processes that increase transcription of a gene or translation of mRNA. Gene upregulation includes any detectable increase in the production of a gene product. In certain examples, production/expression of a gene product increases by at least 2-fold, for example at least 3-fold or at least 4-fold, as compared to a control (such an amount of gene expression in a normal ovarian cell or an ovarian cancer that is chemosensitive). For example, genes listed in Table 1 with a positive t-value, such as RNASEL, POLH, COL5A1, DUSP1, REV3L, and COL1A1, are upregulated in ovarian cancers that are chemorefractory relative to ovarian cancers that are chemosensitive.

In certain examples, a control is a relative amount of gene or protein expression in a biological sample, such as in an ovarian tissue biopsy obtained from a subject that does not have ovarian cancer or has an ovarian cancer that is chemosensitive. In other examples, a control is relative to a standard or reference value of the gene expression or protein expression expected to be present in a subject who does not have ovarian cancer or from a subject that has an ovarian cancer that is chemosensitive. Reference values can include a range of values, real or relative expected to occur under certain conditions. These values can be compared with experimental values to determine if a given molecule is up-regulated or down-regulated.

### Screening Subjects for Chemoresponsiveness

Methods are disclosed herein for determining if a subject is sensitive to treatment with a chemotherapeutic agent, such as platinum-paclitaxel chemotherapy. Subjects can be screened to determine whether the subject with a tumor, such as ovarian cancer, is chemorefractory or is likely to develop chemoresistance by using the disclosed gene signature profile. For example, the differential expression of six or more of the disclosed chemotherapy sensitivity-related molecules relative to a control/reference value can indicate that the subject is likely not to respond to standard chemotherapy, such as those listed in Table 1, or become resistant to such therapy, such as those listed in Table 5. Thus, the methods can be used to determine if the subject is a candidate for receiving standard chemotherapies or one of the therapies disclosed herein.

In one example, the chemotherapy sensitivity-related molecules are detected in a biological sample. In a particular example, the biological sample is a tumor biopsy, such as an ovarian tumor biopsy. In another example, chemotherapy sensitivity-related molecules are detected in a serum sample, such as chemotherapy sensitivity-related molecules secreted or cell surface molecules that are susceptible to enzymatic cleavage at the cell surface.

In an example, chemoresponsiveness can be screened for by detecting at least six of the disclosed chemotherapy sensitivity-related molecules listed in Tables 1 and 5 or a combination thereof. For example, the method can include detecting at least 10, at least 20, at least 30, at least 40, at least 50, at least 60, at least 70, at least 80, at least 90, at least 100, at least 110, at least 120, or at least 130 of these molecules (for example, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135 or 136). Such molecules include, for instance, nucleic acid sequences (such as DNA, cDNA, or mRNAs) and proteins. Specific genes include those listed in Tables 1 and 5, as well as fragments of the full-length genes, cDNAs, or mRNAs (and proteins encoded thereby).

In particular examples, the method indicates if a subject is chemorefractive. In these examples, the expression of at least 1, at least 6, at least 10, at least 20, at least 30, at least 40, at least 50, at least 60, at least 70, at least 80, at least 90, or at least 100 molecules (for example, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or 105 molecules) indicative of chemorefraction as listed in Table 1 are detected. For example, the method can identify an ovarian tumor as chemorefractory by detecting alterations in expression of at least six chemotherapy sensitivity-related molecules listed in Table 1, such as RNASEL, POLH, COL5A1, DUSP1, REV3L, and COL1A1, wherein increased expression in these six molecules indicates the tumor is chemorefractory.

In other particular examples, the method indicates if an ovarian tumor is chemoresistant by detecting at least 1, at least 6, at least 10, at least 20, or at least 30 (for example, 2, 3, 4, 5, 6, 7, 8, 9, 10, 11, 12, 13, 14, 15, 16, 17, 18, 19, 20, 25, 30, or 31) of the molecules listed in Table 5. For example, the method can identify an ovarian tumor as chemoresistant by detecting alterations in expression of at least one chemotherapy sensitivity-related molecule listed in Table 5, such as MARCKS, LOXL1, COL12A1, E2F7 or C5orf13, wherein increased expression in one or more of these molecules indicates the tumor is chemoresistant.

In several examples, the method involves detecting expression of chemotherapy sensitivity-related molecules at either the nucleic acid level or protein level. Certain methods involve determining whether a gene expression profile from the subject indicates chemoresponsiveness by using an array of molecules. For example, the array can include oligonucleotides complementary to all chemotherapy sensitivity-related genes listed in Table 1 and/or Table 5.

In an example, the array includes oligonucleotides complementary to at least one of the disclosed chemotherapy sensitivity-related molecules listed in Tables 1 and 5 or a subset thereof, such as at least 6, at least 10, at least 20, at least 30, at least 40, at least 50, at least 60, at least 70, at least 80, at least 90, at least 100, at least 110, at least 120, or at least 130 molecules (for example, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135 or 136 of those listed). In particular examples, the array includes oligonucleotides complementary to at least 6, at least 10, at least 20, at least 30, at least 40, at least 50, at least 60, at least 70, at least 80, at least 90, or at least 100 molecules (for example, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or 105) indicative of chemorefraction as listed in Table 1. In other particular examples, the array includes oligonucleotides complementary to at least 6, at least 10, at least 20, or at least 30 (for example, 1, 2, 3, 4, 5, 6, 7, 8, 9, 10, 11, 12, 13, 14, 15, 16, 17, 18, 19, 20, 25, 30, or 31) of chemotherapy sensitivity-related molecules indicative of chemoresistance listed in Table 5. However, one skilled in the art will appreciate that an array can include other molecules such as positive or negative controls (e.g., housekeeping genes such as β-actin) and other ovarian cancer markers.

### Detection of Chemotherapy Sensitivity-Related Nucleic Acids

Expression of a nucleic acid in a sample can be detected using routine methods. In some examples, nucleic acids in a biological sample are isolated, amplified, or both, prior to detecting expression. In some examples, amplication and detection of expression occur simultaneously or nearly simultaneously. For example, nucleic acids can be isolated and amplified by employing commercially available kits. In an example, the biological sample can be incubated with primers that permit the amplification of one or more of the disclosed chemotherapy sensitivity-related mRNAs, under conditions sufficient to permit amplification of such products. The resulting amplicons can be detected.

In another example, the biological sample is incubated with probes that can bind to one or more of the disclosed chemotherapy sensitivity-related molecule nucleic acid sequences (such as cDNA, genomic DNA, or RNA (such as mRNA)) under high stringency conditions. The resulting hybridization can then be detected using methods known in the art.

In other examples, a subject is screened by applying isolated nucleic acid molecules obtained from a biological sample including ovarian cancer cells to an array. In such example, the array includes oligonucleotides complementary to all chemotherapy sensitivity-related genes listed in Tables 1 and 5 or a subset thereof, such as at least 6, 20, 50 or 100 of the genes listed. In a particular example, the array is a commercially available array such as a U133 Plus 2.0 oligonucleotide array from AFFYMETRIX® (AFFYMETRIX®, Santa Clara, Calif.).

In an example, the isolated nucleic acid molecules are incubated with the array including oligonucleotides complementary to the chemotherapy sensitivity-related molecules listed in Tables 1 and 5 for a time sufficient to allow hybridization between the isolated nucleic acid molecules and oligonucleotide probes, thereby forming isolated nucleic acid molecule:oligonucleotide complexes. The isolated nucleic acid molecule:oligonucleotide complexes are then analyzed to determine if expression of the isolated nucleic acid molecules is altered. The presence of differential expression in at least 6, at least 10, at least 20, at least 30, at least 40, at least 50, at least 60, at least 70, at least 80, at least 90, at least 100, at least 110, at least 120, or at least 130 molecules listed in Table 1 and/or Table 5 (for example, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135 or 136 of those listed) indicates that the ovarian cancer cells have a decreased sensitivity to a chemotherapeutic agent.

In a particular example, expression is detected in at least 6, at least 10, at least 20, at least 30, at least 40, at least 50, at least 60, at least 70, at least 80, at least 90, or at least 100 molecules listed in Table 1 (for example, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or 105 molecules) of the chemotherapy sensitivity-related molecules indicative of chemorefraction as listed in Table 1. In this example, the presence of differential expression in these chemotherapy sensitivity-related molecules indicates that the ovarian cancer cells are chemorefactory to chemotherapy treatment. In a further example, the at least six genes include RNASEL, POLH, COL5A1, DUSP1, REV3L and COL1A1 which are all up-regulated in subjects with chemorefractory ovarian cancer.

In other particular examples, differential expression is detected in at least 6, at least 10, at least 20, or at least 30 molecules (for example, 7, 8, 9, 10, 11, 12, 13, 14, 15, 16, 17, 18, 19, 20, 25, 30, or 31 molecules) that are indicative of chemoresistance and are represented by any of the molecules listed in Table 5. In this example, the presence of differential expression of at least six chemotherapy sensitivity-related molecules indicates that the ovarian cancer cells are resistant to a chemotherapeutic agent.

### Detecting Chemotherapy-Sensitivity Related Proteins

As an alternative or in addition to detecting nucleic acids, proteins can be detected. using routine methods such as Western blot or mass spectrometry. In some examples, proteins are purified before detection. In one example, chemotherapy sensitivity-related proteins can be detected by incubating the biological sample with an antibody that specifically binds to one or more of the disclosed chemotherapy sensitivity-related proteins encoded by the genes listed in Table 1 and/or Table 5. The primary antibody can include a detectable label. For example, the primary antibody can be directly labeled, or the sample can be subsequently incubated with a secondary antibody that is labeled (for example with a fluorescent label). The label can then be detected, for example by microscopy, ELISA, flow cytometery, or spectrophotometry. In another example, the biological sample is analyzed by Western blotting for the presence of at least one of the disclosed chemotherapy sensitivity-related molecules (see Tables 1 and 5).

In one example, the antibody that specifically binds a chemotherapy sensitivity-related molecule (such as those listed in Tables 1 and 5) is directly labeled with a detectable label. In another example, each antibody that specifically binds a chemotherapy sensitivity-related molecule (the first antibody) is unlabeled and a second antibody or other molecule that can bind the human antibody that specifically binds the respective chemotherapy sensitivity-related molecule is labeled. As is well known to one of skill in the art, a second antibody is chosen that is able to specifically bind the specific species and class of the first antibody. For example, if the first antibody is a human IgG, then the secondary antibody can be an anti-human-IgG. Other molecules that can bind to antibodies include, without limitation, Protein A and Protein G, both of which are available commercially.

Suitable labels for the antibody or secondary antibody include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary magnetic agent is gadolinium, and non-limiting exemplary radioactive labels include ¹²⁵I, ¹³¹I, ³⁵_{S} or ³_{H}.

In an alternative example, chemotherapy sensitivity-related molecules can be assayed in a biological sample by a competition immunoassay utilizing chemotherapy sensitivity-related molecule standards labeled with a detectable substance and an unlabeled antibody that specifically binds the desired chemotherapy sensitivity-related molecule. In this assay, the biological sample (such as serum, tissue biopsy, or cells isolated from a tissue biopsy), the labeled chemotherapy sensitivity-related molecule standards and the antibody that specifically binds the desired chemotherapy sensitivity-related molecule are combined and the amount of labeled chemotherapy sensitivity-related molecule standard bound to the unlabeled antibody is determined. The amount of chemotherapy sensitivity-related molecule in the biological sample is inversely proportional to the amount of labeled chemotherapy sensitivity-related molecule standard bound to the antibody that specifically binds the chemotherapy sensitivity-related molecule.

In some examples, a subject is screened by detecting protein expression. In one example, a subject is screened by determining whether they have differential expression of one or more of the disclosed chemotherapy sensitivity-related molecules. For example, a subject is screened to determine whether they have increased levels of one or more of the disclosed chemotherapy sensitivity-related molecules that is upregulated in chemoresistant or chemorefractory ovarian cancers in their serum (for example relative to a level present in a serum sample from a subject no having a tumor or having a chemosensitive ovarian cancer), for example using an antibody that specifically binds one or more of the disclosed chemotherapy sensitivity-related molecule (such as those described below).

### Comparing Detected Chemotherapy-Sensitivity Related Molecules to Reference or Control Values

The expression of chemotherapy-sensitivity related molecules can be compared to a reference value or control sample to determine if there is differential expression of the detected molecules. In one example, the expression of chemotherapy-sensitivity related molecules detected in a test sample is compared to a reference value, such as an amount of the given gene or protein expected to be expressed in an ovarian cell obtained from a subject who does not have ovarian cancer or who has ovarian cancer that is chemoresponsive. In other examples, the expression level of one or more chemotherapy-sensitivity related molecules is compared to a control sample, such as a sample obtained from a subject who does not have ovarian cancer or who has a chemoresponsive ovarian cancer.

### Methods of Identifying Chemosensitivity Altering Agents

This disclosure has shown, among other things, that differential expression of chemotherapy sensitivity-related molecules can be used to identify ovarian tumors that are chemosensitive, chemoresistant or chemorefractory. This discovery permits, for instance, methods for identifying agents that alter the chemoresponsiveness of a tumor. In specific examples, the method includes identifying an agent that alters activity (including expression) of one or more of the chemotherapy sensitivity-related molecules listed in Table 1 and/or Table 5. For example, genes that are upregulated in ovarian cancers that are chemorefactory (Table 1, with a positive t-value) can be used to screen for agents that reduce or inhibit this expression or activity. In contrast, genes that are downregulated in ovarian cancers that are chemorefractory (Table 1, with a negative t-value) can be used to screen for agents that increase this expression or activity. Such identified agents can be used to treat chemorefractory or chemoresistant ovarian cancers.

In one example, a chemosensitivity altering agent is identified by contacting a tumor cell, such as an ovarian cancer cell with one or more test agents under conditions sufficient for the one or more test agents to alter the activity of chemotherapy sensitivity-related molecules, such as those listed in Table 1 and/or Table 5. In some examples, multiple chemotherapy sensitivity-related molecules in Tables 1 and 5 are screened, such as at least 6, at least 20, or at least 100 of those shown can be assayed in the presence of the test agents. For example, expression of at least six chemotherapy sensitivity-related molecules are detected in the presence and absence of one or more test agents, such as at least six test agents, and the expression levels are compared whereby the presence of differential expression of the chemotherapy sensitivity-related molecules in the presence/absence of the agents indicates that the test agents alter the activity (such as expression level) of such molecules. The one or more test agents can be any substance, including, but not limited to, a protein (such as an antibody), nucleic acid molecule (such as a siRNA), organic compound, inorganic compound, or other molecule of interest. In a particular example, the test agent is a siRNA or antibody specific for any of the disclosed chemotherapy sensitivity-related molecules listed in Tables 1 and 5 that are overexpressed in chemoresistant or chemorefractory ovarian tumors. In some examples, such siRNAs or antibodies decrease the expression or activity of these chemotherapy sensitivity-related molecules. The test agenst can be contacted with an ovarian cancer cell in vitro or in vivo (e.g., by administrating the test agent to a laboratory animal model for ovarian cancer). Agents that reverse the undesired expression or activity can be selected for further study.

In one specific example, the one or more test agent alters the activity (such as the expression level) of at least 1, at least 6, at least 10, at least 20, at least 30, at least 40, at least 50, at least 60, at least 70, at least 80, at least 90, or at least 100 (for example, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 or 105) chemotherapy sensitivity-related molecules associated with chemorefraction listed in Table 1.

In other examples, the one or more test agent alters the activity of at least 1, at least 6, at least 10, at least 20, or at least 30 (for example, 2, 3, 4, 5, 6, 7, 8, 9, 10, 11, 12, 13, 14, 15, 16, 17, 18, 19, 20, 25, 30, or 31) chemotherapy sensitivity-related molecules associated with chemoresistance listed in Table 5.

A. Agents

Any agent that has potential (whether or not ultimately realized) to alter chemotherapy sensitivity-related molecule expression (for instance in ovarian tumor cells), affect a chemotherapy sensitivity-related molecule function (such as, decrease chemotherapy sensitivity-related molecule-dependent resistance to chemotherapy), affect the interaction (in vivo or in vitro) between chemotherapy sensitivity-related molecule and one or more of its signal transduction pathway member molecules (such as, its specific binding partners) or otherwise be a chemotherapy sensitivity-related molecule mimetic is contemplated for use in the methods of this disclosure. Such agents may include, but are not limited to, siRNAs, peptides such as for example, soluble peptides, including but not limited to members of random peptide libraries (see, e.g., Lam et al., Nature, 354:82-84, 1991; Houghten et al., Nature, 354:84-86, 1991), and combinatorial chemistry-derived molecular library made of D- and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab′)₂ and Fab expression library fragments, and epitope-binding fragments thereof), and small organic or inorganic molecules (such as so-called natural products or members of chemical combinatorial libraries).

Libraries (such as combinatorial chemical libraries) useful in the disclosed methods include, but are not limited to, peptide libraries (see, e.g., U.S. Pat. No. 5,010,175; Furka, Int. J. Pept. Prot. Res., 37:487-493, 1991; Houghton et al., Nature, 354:84-88, 1991; PCT Publication No. WO 91/19735), encoded peptides (e.g., PCT Publication WO 93/20242), random bio-oligomers (e.g., PCT Publication No. WO 92/00091), nucleic acid libraries (see Sambrook et al. Molecular Cloning, A Laboratory Manual, Cold Springs Harbor Press, N.Y., 1989; Ausubel et al., Current Protocols in Molecular Biology, Green Publishing Associates and Wiley Interscience, N.Y., 1989), peptide nucleic acid libraries (see, e.g., U.S. Pat. No. 5,539,083), antibody libraries (see, e.g., Vaughn et al., Nat. Biotechnol., 14:309-314, 1996; PCT App. No. PCT/US96/10287), carbohydrate libraries (see, e.g., Liang et al., Science, 274:1520-1522, 1996; U.S. Pat. No. 5,593,853), small organic molecule libraries and the like.

Libraries useful for the disclosed screening methods can be produced in a variety of manners including, but not limited to, spatially arrayed multipin peptide synthesis (Geysen, et al., Proc. Natl. Acad. Sci., 81(13):3998-4002, 1984), “tea bag” peptide synthesis (Houghten, Proc. Natl. Acad. Sci., 82(15):5131-5135, 1985), phage display (Scott and Smith, Science, 249:386-390, 1990), spot or disc synthesis (Dittrich et al., Bioorg. Med. Chem. Lett., 8(17):2351-2356, 1998), or split and mix solid phase synthesis on beads (Furka et al., Int. J. Pept. Protein Res., 37(6):487-493, 1991; Lam et al., Chem. Rev., 97(2):411-448, 1997). Libraries may include a varying number of compositions (members), such as up to about 100 members, such as up to about 1000 members, such as up to about 5000 members, such as up to about 10,000 members, such as up to about 100,000 members, such as up to about 500,000 members, or even more than 500,000 members.

In one embodiment, high throughput screening methods involve providing a nucleic acid (e.g., RNAi) or antibody library containing a large number of potential therapeutic compounds (e.g., potential chemoresponsiveness altering agents, chemotherapy sensitivity-related molecule mimetics, or affectors of chemotherapy sensitivity-related molecule-signal transduction molecule interaction). Such libraries are then screened in one or more assays as described herein to identify those library members (particularly chemical species or subclasses) that display a desired characteristic activity (such as decreasing chemotherapy sensitivity-related molecule expression, affecting chemotherapy sensitivity-related molecule signal transduction pathway, or specific binding to a chemotherapy sensitivity-related molecule-specific antibody). The compounds thus identified can serve as conventional “lead compounds” or can themselves be used as potential or actual therapeutics. In some instances, pools of candidate agents may be identified and further screened to determine which individual or subpools of agents in the collective have the desired activity.

B. Assays

Screening methods may include, but are not limited to, methods employing solid phase, liquid phase, cell-based or virtual (in silico) screening assays. In some exemplary assays, compounds that affect the expression or a function of chemotherapy sensitivity-related molecule (such as decrease expression or activity of chemotherapy sensitivity-related molecules upregulated in chemoresistant or chemorefractory ovarian tumors) are identified. For instance, certain assays may identify compounds that bind to chemotherapy sensitivity-related molecule gene regulatory sequences (e.g., promoter sequences) and which may modulate chemotherapy sensitivity-related molecule gene expression (e.g., decrease expression or activity of such molecules that are overexpressed in chemoresistant or chemorefractory ovarian tumors or increase expression or activity of those molecules down-regulated in said samples). Other representative assays identify compounds that interfere with or otherwise affect a protein-protein interaction between chemotherapy sensitivity-related molecule and one or more of its signal transduction pathway members (such as a specific binding partners), or compounds that are specifically recognized by an anti-chemotherapy sensitivity-related molecule antibody (such as an antibody specific for a chemotherapy sensitivity-related molecule). Compounds identified via assays such as those described herein may be useful, for example, for treating ovarian cancer or to design and/or further identify ovarian cancer treatments.

1. Agents that Modulate the Expression of a Chemotherapy Sensitivity-Related Molecule Gene, Transcript or Polypeptide

Also disclosed herein are methods of identifying agents that modulate the expression of a chemotherapy sensitivity-related molecule polypeptide or a nucleic acid encoding it (such as a chemotherapy sensitivity-related molecule gene or transcript). Generally, such methods involve contacting (directly or indirectly) with a test agent an expression system comprising a nucleic acid sequence encoding a chemotherapy sensitivity-related molecule polypeptide, or a reporter gene operably linked to a chemotherapy sensitivity-related molecule transcription regulatory sequence, and detecting a change (e.g., a decrease or increase) in the expression of the chemotherapy sensitivity-related molecule-encoding nucleic acid or reporter gene. “Test agent” as used herein include all agents (and libraries of agents) described above.

Modulation of the expression of a chemotherapy sensitivity-related molecule gene or gene product (e.g., transcript or protein) can be determined using any expression system capable of expressing a chemotherapy sensitivity-related molecule polypeptide or transcript (such as a cell, tissue, or organism, or in vitro transcription or translation systems). In some embodiments, cell-based assays are performed. Non-limiting exemplary cell-based assays may involve test cells such as cells (including cell lines) that normally express a chemotherapy sensitivity-related molecule gene, its corresponding transcript(s) and/or chemotherapy sensitivity-related molecule protein(s), or cells (including cell lines) that have been transiently transfected or stably transformed with a reporter construct driven by a regulatory sequence of a chemotherapy sensitivity-related molecule gene.

As mentioned above, some disclosed methods involve cells (including cell lines) that have been transiently transfected or stably transformed with a reporter construct driven by a regulatory sequence of a chemotherapy sensitivity-related molecule gene. A “regulatory sequence” as used herein can include some or all of the regulatory elements that regulate the expression of a particular nucleic acid sequence (such as a chemotherapy sensitivity-related molecule gene) under normal circumstances. In particular examples, a regulatory region includes the contiguous nucleotides located at least 100, at least 500, at least 1000, at least 2500, at least 5000, or at least 7500 nucleotides upstream of the transcriptional start site of the regulated nucleic acid sequence (such as a chemotherapy sensitivity-related molecule gene).

In method embodiments involving a cell transiently or stably transfected with a reporter construct operably linked to a chemotherapy sensitivity-related molecule gene regulatory region, the level of the reporter gene product can be measured. Reporter genes are nucleic acid sequences that encode readily assayed proteins. Numerous reporter genes are commonly known and methods of their use are standard in the art. Non-limiting representative reporter genes are luciferase, β-galactosidase, chloramphenicol acetyl transferase, alkaline phosphatase, green fluorescent protein, and others. In the applicable methods, the reporter gene product is detected using standard techniques for that particular reporter gene product (see, for example, manufacturer's directions for human placental alkaline phosphatase (SEAP), luciferase, or enhance green fluorescent protein (EGPF) available from BDBiosciences (Clontech); or galactosidase/luciferase, luciferase, or galactosidase available from Applied Biosystems (Foster City, Calif., USA); or available from various other commercial manufacturers of reporter gene products). A difference in the level and/or activity of reporter gene measure in cells in the presence or absence of a test agent indicates that the test agent modulates the activity of the chemotherapy sensitivity-related molecule regulatory region driving the reporter gene.

A change in the expression of a chemotherapy sensitivity-related molecule gene (or a reporter gene), transcript or protein can be determined by any method known in the art. For example, the levels of a chemotherapy sensitivity-related molecule (or reporter gene) transcript or protein can be measured by standard techniques, such as for RNA, Northern blot, PCR (including RT-PCR or q-PCR), in situ hybridization, or nucleic acid microarray, or, for protein, Western blot, antibody array, or immunohistochemistry. Alternatively, test cells can be examined to determine whether one or more cellular phenotypes have been altered in a manner consistent with modulation of expression of chemotherapy sensitivity-related molecule.

2. Agents that Affect the Interaction Between Chemotherapy Sensitivity-Related Molecules and Their Signal Transduction Pathway Members

Differential expression of one or more of the disclosed chemotherapy sensitivity-related molecules may result in alterations of the signal transduction pathway member molecules regulated by the chemotherapy sensitivity-related molecules. Agents that affect an interaction between chemotherapy sensitivity-related molecule and one or more of its signal transduction family members can be identified by a variety of assays, including solid-phase or solution-based assays. In a solid-phase assay, a chemotherapy sensitivity-related molecule polypeptide (as described in detail elsewhere in this specification) and one or more chemotherapy sensitivity-related signal transduction molecules are mixed under conditions in which chemotherapy sensitivity-related molecule and its signaling molecule(s) normally interact. One of the molecules (e.g., a chemotherapy sensitivity-related molecule polypeptide or its specific signaling transduction molecule(s)) is labeled with a marker such as biotin, fluoroscein, EGFP, or enzymes to allow easy detection of the labeled component. The unlabeled binding partner is adsorbed to a support, such as a microtiter well or beads. Then, the labeled binding partner is added to the environment where the unlabeled molecule is immobilized under conditions suitable for interaction between the two molecules. One or more test compounds, such as compounds in one or more of the above-described libraries, are separately added to individual microenvironments containing the interacting molecules. Agents capable of affecting the interaction between such molecules are identified, for instance, as those that enhance retention or binding of the signal (i.e., labeled molecule) in the reaction microenvironment, for example, in a microtiter well or on a bead for example. As discussed previously, combinations of agents can be evaluated in an initial screen to identify pools of agents to be tested individually, and this process is easily automated with currently available technology.

In still other methods, solution phase selection can be used to screen large complex libraries for agents that specifically affect protein-protein interactions (see, e.g., Boger et al., Bioorg. Med. Chem. Lett., 8(17):2339-2344, 1998); Berg et al., Proc. Natl. Acad. Sci., 99(6):3830-3835, 2002). In this example, each of two proteins that are capable of physical interaction (for example, chemotherapy sensitivity-related molecule and one of its respective signal transduction molecules) are labeled with fluorescent dye molecule tags with different emission spectra and overlapping adsorption spectra. When these protein components are separate, the emission spectrum for each component is distinct and can be measured. When the protein components interact, fluorescence resonance energy transfer (FRET) occurs resulting in the transfer of energy from a donor dye molecule to an acceptor dye molecule without emission of a photon. The acceptor dye molecule alone emits photons (light) of a characteristic wavelength. Therefore, FRET allows one to determine the kinetics of two interacting molecules based on the emission spectra of the sample. Using this system, two labeled protein components are added under conditions where their interaction resulting in FRET emission spectra. Then, one or more test compounds, such as compounds in one or more of the above-described libraries, are added to the environment of the two labeled protein component mixture and emission spectra are measured. An increase in the FRET emission, with a concurrent decrease in the emission spectra of the separated components indicates that an agent (or pool of candidate agents) has affected (e.g., enhanced) the interaction between the protein components.

Interactions between chemotherapy sensitivity-related molecule and one or more of its specific signal transduction family members also can be determined (e.g., quantitatively or qualitatively) by co-immunoprecipitation of the relevant component polypeptides (e.g., from cellular extracts), by GST-pull down assay (e.g., using purified GST-tagged bacterial proteins), and/or by yeast two-hybrid assay, each of which methods is standard in the art. Conducting any one or more such assays in the presence and, optionally, absence of a test compound can be used to identify agents that affect the chemotherapy sensitivity-related molecule:specific signal transduction member interaction in the presence of the test compound as compared to in the absence of the test compound or as compared to some other standard or control. In particular methods, the formation of a chemotherapy sensitivity-related molecule:specific-signal transduction member complex is decreased or inhibited when the amount of such complex is at least 20%, at least 30%, at least 50%, at least 100% less than a control measurement (e.g., in the same test system prior to addition of a test agent, or in a comparable test system in the absence of a test agent). In some methods, inhibition of a chemotherapy sensitivity-related molecule:specific-signal transduction memberr interaction may be nearly complete such that substantially no protein-protein complex involving chemotherapy sensitivity-related molecule and that particular specific binding partner is detected using traditional detection methods. In other methods, the formation of a chemotherapy sensitivity-related molecule:specific-signal transduction member complex is increased or enhanced when the amount of such complex is at least 20%, at least 30%, at least 50%, at least 100% or at least 250% higher than a control measurement (e.g., in the same test system prior to addition of a test agent, or in a comparable test system in the absence of a test agent).

3. Identifying Agents that Affects a chemotherapy sensitivity-related molecule Function/Activity

Chemotherapy sensitivity-related molecule differential expression can regulate ovarian tumor responsiveness to chemotherapy. Accordingly, it is desirable to identify agents having the potential to alter one or more of these chemotherapy sensitivity-related molecule functions/activities (e.g., inhibit biological activity of up-regulated chemotherapy sensitivity-related molecules in chemoresistant/chemorefractory ovarian tumors or increase biological activity of those molecules downregulated in chemoresistant/chemorefractory ovarian tumors), at least, because such agents are candidates for ovarian cancer therapeutics.

As previously described, an alteration in the activity of one or more of the disclosed chemotherapy sensitivity-related molecules includes an increase or decrease in production of a gene product, such as RNA or protein. For example, an alteration can include processes that downregulate or decrease transcription of a gene or translation of mRNA. Gene downregulation includes any dectable decrease in the production of a gene product. In certain examples, production/expression of a gene product decreases by at least 2-fold, for example at least 3-fold or at least 4-fold, as compared to a control (such an amount of gene expression in a normal cell or a chemosensitive ovarian cancer cell or an amount of expression in absence of the test agent). In one example, a control is a relative amount of gene expression or protein expression in a biological sample (e.g., ovarian sample) obtained from a subject who does not have ovarian cancer or has a chemosensitive ovarian cancer.

In another example, an alteration can include processes that increase transcription of a gene or translation of mRNA. Gene upregulation includes any detectable increase in the production of a gene product. In certain examples, production/expression of a gene product increases by at least 2-fold, for example at least 3-fold or at least 4-fold, as compared to a control (such an amount of gene expression in a normal cell or a chemosensitive ovarian cancer cell or an amount of expression in absence of the test agent). In one example, a control is a relative amount of gene expression in a biological sample, such as in an ovarian tissue biopsy obtained from a subject that does not have ovarian cancer or has an ovarian cancer that is chemosensitive.

Exemplary assays to identify such agents can involve detecting a chemotherapy sensitivity-related molecule-dependent functional (e.g., phenotypic) difference in an in vitro or in vivo assay system. In these embodiments, the assay system is capable of undergoing the desired phenotypic change, e.g., increasing responsiveness to chemotherapy. Accordingly, certain cell-based systems are suitable for conducting such assays. In particular embodiments, the same type of cell is used for test and control assay systems.

To ensure that an observed phenotype is attributable to a chemotherapy sensitivity-related molecule polypeptide that is upregulated in ovarian cancers that are chemoresistant or chemorefractory, a control assay system will express substantially no chemotherapy sensitivity-related molecule (e.g., undetectable by Western blot) or substantially less chemotherapy sensitivity-related molecule as compared to a non-control assay system. In this context, substantially less means at least 25% less, at least 50% less, at least 75%, or at least 90% less chemotherapy sensitivity-related molecule in the control versus non-control assay system. A non-control assay system expresses or overexpresses chemotherapy sensitivity-related molecule (or otherwise is treated to have more chemotherapy sensitivity-related molecule) as compared to control (e.g., at least 10%, at least 25%, at least 50%, at least 75%, or at least 90% more chemotherapy sensitivity-related molecule expression than control). In some examples, such expression or overexpression is achieved by transfecting one or more cells with an expression vector encoding the chemotherapy sensitivity-related molecule polypeptide. In some examples, a GST-chemotherapy sensitivity-related molecule fusion protein can be expressed either in a transfected cell or transgenic animal. The GST module of such fusion protein permits rapid identification of chemotherapy sensitivity-related molecule-expressing cells.

One or more test agents are contacted to the control and non-control assay systems (e.g., cells of such assay systems), and a chemotherapy sensitivity-related molecule-dependent phenotype (such as responsiveness to chemotherapy) is detected. An agent having potential to reduce or inhibit chemoresistance/chemorefraction is one for which chemoresponsiveness is greater in the non-control, chemotherapy sensitivity-related molecule expressing or overexpressing system. For instance, in one specific non-limiting example, GFP-positive chemotherapy sensitivity-related molecule-overexpressing ovarian tumor cells are isolated from transgenic mice (e.g., expressing a heterologous GFP-chemotherapy sensitivity-related molecule fusion protein) are cultured on in the presence of test compounds or vehicle. Compounds are identified that enhance or attenuate chemotherapy sensitivity-related molecule-dependent chemoresponsiveness in ovarian tumor cells when compared to control cells (ovarian tumor cells receiving only vehicle). The GFP marker permits this assay to be used in a high-throughput automatic screening format using an imaging system.

In some cell-based method embodiments described here and throughout the specification, test cells or test agents can be presented in a manner suitable for high-throughput screening; for example, one or a plurality of test cells can be seeded into wells of a microtitre plate, and one or a plurality of test agents can be added to the wells of the microtitre plate. Alternatively, one or a plurality of test agents can be presented in a high-throughput format, such as in wells of microtitre plate (either in solution or adhered to the surface of the plate), and contacted with one or a plurality of test cells under conditions that, at least, sustain the test cells. Test agents can be added to test cells at any concentration that is not lethal to the cells. It is expected that different test agents will have different effective concentrations. Thus, in some methods, it is advantageous to test a range of test agent concentrations.

In particular methods, a function of a chemotherapy sensitivity-related molecule polypeptide that is upregulated in ovarian cancers that are chemoresistant or chemorefractory is reduced or inhibited when a quantitative or qualitative measure of such function is at least 20%, at least 30%, at least 50%, at least 100% or at least 250% less than a control measurement (e.g., in the same test system prior to addition of a test agent, in a comparable test system in the absence of a test agent or in test system treated with vehicle alone).

### Methods of Treatment

It is shown herein that chemotherapy sensitivity is associated with differential expression of chemotherapy sensitivity-related molecules. For example, the disclosed gene expression profile has identified one hundred and five chemotherapy sensitivity-related molecules associated with chemorefractory disease and thirty-one chemotherapy sensitivity-related molecules associated with chemoresistance. Based on these observations, methods of treatment to alter sensitivity to a chemotherapeutic agent, such as chemorefraction or chemoresistance associated with ovarian cancer, are disclosed.

Methods are disclosed herein for treating chemoresistance or chemorefraction, such as that associated with treating cancer with a chemotherapeutic agent. In some examples, the method includes determining if the subject has an ovarian tumor that is chemoresistant or chemorefractory (e.g., any methods provided herein). If negative, the subject is chemosensitive and standard chemotherapy can be administered. If the subject has an ovarian tumor that is chemoresistant or chemorefractory, then agents can be administered to reverse the pattern of expression of one or more of the genes/proteins associated with the chemoresistance/chemorefraction. In some examples, a therapy for treating chemorefraction/chemoresistance is selected and then administered.

In one example, the method includes administering a therapeutically effective amount of a composition to a subject who is chemoresistant/chemorefractory that includes a specific binding agent that preferentially binds to one or more chemotherapy sensitivity-related molecules listed in Tables 1 and 5 or a subset thereof, such as at least 1, at least 2, at least 3, at least 5, at least 6, at least 10, at least 20, at least 30, at least 40, at least 50, at least 60, at least 70, at least 80, at least 90, at least 100, at least 110, at least 120, or at least 130 (for example, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135 or 136 of those listed). Such chemotherapy sensitivity-related molecules include, for instance, nucleic acid sequences (such as DNA, cDNA, or mRNAs) and proteins. Specific genes include those listed in Tables 1 and 5, as well as fragments of the full-length genes, cDNAs, or mRNAs (and proteins encoded thereby) whose expression is altered (such as upregulated or downregulated) in ovarian cancer.

In particular examples, the one or more chemotherapy sensitivity-related nucleic acids or proteins include those listed in Table 1 (such as RNASEL, POLH, COL5A1, DUSP1, REV3L, or COL1A1) and are indicative of chemorefraction. In other particular examples, the one or more chemotherapy sensitivity-related molecules include those listed in Table 5 and are indicative of chemoresistance. In certain examples, chemotherapy sensitivity-related molecules whose expression is upregulated or downregulated in ovarian cancer include sequences related to collagens, apoptosis, cell survival and DNA repair genes, such as those listed in Tables 2 and 7. The specific binding agent can be an inhibitor such as a siRNA or an antibody to one or more of the chemotherapy sensitivity-related molecules, for example, to decrease expression or activity of a gene/protein that is increased in chemoresistance/chemorefraction. The specific binding agent can also be an agonist, for example to increase expression or activity of a gene/protein that is decreased in chemoresistance/chemorefracton.

### Increasing Sensitivity to a Chemotherapeutic Agent by Regulating a Chemotherapy Sensitivity-related Molecule

Chemoresistance is a complex phenomenon that involves a change in the expression and biological activity of several genes or gene products. For example, the genes or gene families that are expressed differentially in chemoresistant or chemorefractory subjects can be used as molecular targets for agents allowing a subject's sensitivity/responsiveness to a chemotherapeutic agent to be increased.

In an example, inhibiting chemotherapy sensitivity-related molecules that are up-regulated in chemorefractory or chemoresistant tumors can be used to treat a tumor. Inhibition of a chemotherapy sensitivity-related molecule does not require 100% inhibition, but can include at least a reduction if not a complete inhibition of cell growth or differentiation associated with a specific pathological condition. Treatment of a tumor by reducing the acitivty or expression of chemorefractory or chemoresistant molecules can include delaying the development of the tumor in a subject (such as preventing metastasis of a tumor) by increasing the responsiveness of the tumor to the given chemotherapeutic agent. Treatment of a tumor also includes reducing signs or symptoms associated with the presence of such a tumor (for example by reducing the size or volume of the tumor or a metastasis thereof) by increasing the responsiveness of the tumor to the given chemotherapeutic agent. Such reduced growth can in some examples decrease or slow metastasis of the tumor, or reduce the size or volume of the tumor by at least 10%, at least 20%, at least 50%, or at least 75%. For example, chemotherapy sensitivity-related molecules up-regulated in chemorefractory or chemoresist samples can be inhibited to treat ovarian cancer by increasing the responsiveness of the ovarian cancer to a chemotherapeutic agent, such as a platinum-based chemotherapeutic agent (e.g., carboplatin or cisplatin). In another example, inhibition of chemotherapy sensitivity-related molecules increased with chemorefraction or chemoresistance includes reducing the invasive activity of the tumor in the subject, for example by reducing the ability of the tumor to metastasize by increasing the responsiveness of the tumor to a given chemotherapeutic agent.

In some examples, treatments can include using activators, such as agonists, which increase the activity or expression of chemosensitivity-related molecules that are down-regulated in chemoresistant or chemorefractory tumors. Increasing the activity or expression of a chemotherapy sensitivity-related molecule can include delaying the development of the tumor in a subject (such as preventing metastasis of a tumor) by increasing the responsiveness of the tumor to the given chemotherapeutic agent. Treatment of a tumor also includes reducing signs or symptoms associated with the presence of such a tumor (for example by reducing the size or volume of the tumor or a metastasis thereof) by increasing the responsiveness of the tumor to the given chemotherapeutic agent. Such reduced growth can in some examples decrease or slow metastasis of the tumor, or reduce the size or volume of the tumor by at least 10%, at least 20%, at least 50%, or at least 75%. For example, chemotherapy sensitivity-related molecules down-regulated in chemorefractory or chemoresist samples can be activated to treat ovarian cancer by increasing the responsiveness of the ovarian cancer to a chemotherapeutic agent, such as a platinum-based chemotherapeutic agent (e.g., carboplatin or cisplatin). In another example, activation of chemotherapy sensitivity-related molecules down-regulated with chemorefraction or chemoresistance includes reducing the invasive activity of the tumor in the subject, for example by reducing the ability of the tumor to metastasize by increasing the responsiveness of the tumor to a given chemotherapeutic agent.

In some examples, treatment using the methods disclosed herein prolongs the time of survival of the subject.

### Specific Binding Agents

Specific binding agents are agents that bind with higher affinity to a molecule of interest, than to other molecules. For example, a specific binding agent can be one that binds with high affinity to one of the genes or gene products of the chemotherapy sensitivity-related molecules listed in Tables 1 and 5, but does not substantially bind to another gene or gene product. In a specific example, a specific binding agent binds to one or more genes listed in Tables 1 and 5 which is upregulated thereby reducing or inhibiting expression of the one or more genes. For example, the agent interfers with gene expression (transcription, processing, translation, post-translational modification), such as, by interfering with the gene's mRNA and blocking translation of the gene product or by post-translational modification of a gene product, or by causing changes in intracellular localization. In another specific example, a specific binding agent binds to a protein encoded by of one of the genes listed in Tables 1 and 5 with a binding affinity in the range of 0.1 to 20 nM. In one example, the specific binding agent is an antagonist and is used to inhibit the activity or expression of a chemotherapy sensitivity-related molecule that is up-regulated in a chemorefractory or chemoresistant ovarian tumor. In other examples, the specific binding agent is an agonist that stimulates the activity or expression of a chemotherapy sensitivity-related molecule that is down-regulated in a chemorefractory or chemoresistant ovarian tumor.

Examples of specific binding agents include, but are not limited to siRNA, antibodies, ligands, recombinant proteins, peptide mimetics, and soluble receptor fragments. One specific example of a specific binding agent is a siRNA. Methods of making siRNA that can be used clinically are known in the art. Particular siRNAs and methods that can be used to produce and administer them are described in detail below. In a particular example, siRNA hybridize to REV3L or POLH with high specificity, such as SEQ ID NOS:2-6, 8, 9, 11 and 12.

Another specific example of a specific binding agent is an antibody, such as a monoclonal or polyclonal antibody. Methods of making antibodies that can be used clinically are known in the art. Particular antibodies and methods that can be used to produce them are described in detail below.

In a further example, small molecular weight inhibitors or antagonists of the receptor protein can be used to regulate chemosensitivity. In a particular example, small molecular weight inhibitors or antagonists of the proteins encoded by the genes listed in Tables 1 and 5 are employed.

Specific binding agents can be therapeutic, for example by reducing or inhibiting the biological activity of a nucleic acid or protein whose activity is detrimental. For example, a specific binding agent that binds with high affinity to a gene listed in Tables 1 and 5, may substantially reduce the biological function of the gene or gene product (for example, the ability of the gene or gene product to impart chemorefraction or chemoresistance, to a tumor cell, respectively). In other examples, a specific binding agent that binds with high affinity to one of the proteins encoded by the genes listed in Tables 1 and 5, may substantially reduce the biological function of the protein (for example, the ability of the protein to promote chemorefraction or chemoresistance, respectively). Such agents can be administered in therapeutically effective amounts to subjects in need thereof, such as a subject having ovarian cancer, such as papillary serous ovarian cancer that is chemorefractory or chemoresistant.

### Pre-Screening Subjects

In some examples, subjects are initially screened to determine if they are likely to respond to chemotherapy by use of the disclosed gene expression profile (as discussed in detail above). For example, the disclosed gene expression profile can be used to determine if a subject with ovarian cancer is likely to be chemorefractory, chemoresistant or chemosensitive. In one example, a subject that is likely to be chemorefractory, chemoresistant or chemosensitive is selected. Subjects that are chemosensitive can receive standard chemotherapy. Subjects that are chemorefractory or chemoresistant can receive any of the therapies disclosed herein.

### Exemplary Tumors

A tumor is an abnormal growth of tissue that results from excessive cell division. A particular example of a tumor is cancer. For example, the current application provides methods for the treatment (such as the prevention or reduction of metastasis) of tumors (such as cancers) by altering a tumor's response to a chemotherapeutic agent. In some examples, the tumor is treated in vivo, for example in a mammalian subject, such as a human subject. Exemplary tumors that can be treated using the disclosed methods include, but are not limited to ovarian cancer, including metastases of such tumors to other organs.

### Administration of Therapeutic Agents

This disclosure contemplates pharmaceutical compositions including one or more chemotherapy sensitivity-related molecule polypeptides and/or one or more nucleic acids encoding such polypeptides, and further contemplates administering chemotherapy sensitivity-related molecule therapeutics to subjects in need thereof, such as to subjects having chemoresistant or chemorefractory ovarian tumors. Delivery systems and treatment regimens useful for such agents are known and can be used to administer these agents as therapeutics. In addition, representative embodiments are described below.

### 1. Administration of Nucleic Acid Molecules

In some embodiments where a therapeutic molecule is a nucleic acid encoding a therapeutic protein or peptide (for example, a nucleic acid molecule encoding a chemotherapy sensitivity-related molecule polypeptide that is downregulated in a chemoresistant or chemorefractory ovarian cancer), or another type of therapeutic nucleic acid molecule (such as an siRNA, anti-sense oligonucleotide, ribozyme or other inhibitory nucleic acid specific for a gene that is upregulated in chemoresistant or chemorefractory ovarian cancer), administration of the nucleic acid may be achieved in a variety of ways. All forms of nucleic acid delivery are contemplated by this disclosure, including, without limitation, synthetic oligos, naked DNA, naked RNA (such as capped RNA), and plasmid or viral vectors (which may or may not be integrated into a target cell genome). For example, an expressible nucleic acid can be administered by use of a viral vector (see U.S. Pat. No. 4,980,286), or by direct injection, or by use of microparticle bombardment (for example, a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci., 88:1864-8, 1991). Alternatively, the expressible nucleic acid can be introduced into a host cell (such as a stem cell, e.g., a stem cell capable of neural differentiation) for expression of a polypeptide therapeutic in the host cell. In some examples, transfected/transformed host cells can be transplanted into a subject. In some instances, a nucleic acid can be incorporated within host cell DNA, for example, by homologous or non-homologous recombination, for stably expressing a therapeutic.

Expression vectors are commonly available that provide, for instance, constitutive, regulated, or cell/tissue-specific expression of a transcribable nucleic acid (e.g., a nucleic acid encoding a chemotherapy sensitivity-related molecule polypeptide) included in the expression vector. All these vectors achieve the basic goal of delivering into the target cell a heterologous nucleic acid sequence and control elements needed for transcription. The vector pcDNA, which includes a strong viral promoter (CMV), is an example of an expression vector for constitutive expression of a heterologous DNA. Certain retroviral vectors (such as pRETRO-ON, Clontech) also use the constitutive CMV promoter but have the advantages of entering cells without any transfection aid, integrating into the genome of target cells only when the target cell is dividing. Regulated expression vectors include control elements that permit expression of an operably linked nucleic acid only when a corresponding regulator molecule (such as tetracycline or steroid hormones) is present. Exemplary regulated vectors include pMAM-neo (Clontech) or pMSG (Pharmacia), which use the steroid-regulated MMTV-LTR promoter, or pBPV (Pharmacia), which includes a metallothionein-responsive promoter. Numerous cell/tissue-specific expression vectors are also available for expression of heterologous nucleic acids in any of a variety of tissues or cell types.

Viral vectors, which are derived from various viral genomes, are similarly numerous and commercially available. Exemplary viral vectors are derived from retroviruses (such as lentivirus), adenovirus, herpes simplex virus (HSV; Margolskee et al., Mol. Cell. Biol. 8:2837-2847, 1988), adeno-associated virus (McLaughlin et al., J. Virol. 62:1963-1973, 1988), polio virus and vaccinia virus (Moss et al., Annu. Rev. Immunol. 5:305-324, 1987). Representative retroviral vectors are derived from lentiviruses, Moloney murine leukemia virus (MoMuLV), Harvey murine sarcoma virus (HaMuSV), murine mammary tumor virus (MuMTV), and Rous Sarcoma Virus (RSV). Multiple teachings concerning viral vectors are available, e.g., Anderson, Science, 226:401-409, 1984; Hughes, Curr. Comm. Mol. Biol., 71:1-12, 1988; Friedman, Science, 244:1275-1281, 1989 and Anderson, Science, 256:608-613, 1992. Some viral vectors are replication-deficient and/or non-infective. Non-limiting representative neurotrophic viral vectors include herpes simplex viral vectors (see, e.g., U.S. Pat. No. 5,673,344) and adenoviral vectors (see, e.g., Barkats et al., Prog. Neurobiol., 55:333-341, 1998), or AAV or lentiviral vectors pseudotyped with rabies-G glycoptroein (Mazarakis et al., Human Mol. Genet., 10:2109-2121, 2001; Azzouz, et al., J. Neurosci., 22:10302-10312, 2002; Azzouz, et al., Nature, 429:413-417, 2004).

Other methods of delivery are also contemplated. For instance, lipidic and liposome-mediated gene delivery has recently been used successfully for transfection with various genes (for reviews, see Templeton and Lasic, Mol. Biotechnol., 11:175 180, 1999; Lee and Huang, Crit. Rev. Ther. Drug Carrier Syst., 14:173-206, 1997; and Cooper, Semin. Oncol., 23:172-187, 1996). For instance, cationic liposomes have been analyzed for their ability to transfect monocytic leukemia cells, and shown to be a viable alternative to using viral vectors (de Lima et al., Mol. Membr. Biol., 16:103-109, 1999). Such cationic liposomes can also be targeted to specific cells through the inclusion of, for instance, monoclonal antibodies or other appropriate targeting ligands (Kao et al., Cancer Gene Ther., 3:250-256, 1996).

### 2. Administration of Polypeptides or Peptides

In some embodiments, therapeutic agents comprising polypeptides or peptides may be delivered by administering to the subject a nucleic acid encoding the polypeptide or peptide, in which case the methods discussed in the section entitled “Administration of Nucleic Acid Molecules” should be consulted. In other embodiments, polypeptide or peptide therapeutic agents may be isolated from various sources and administered directly to the subject. For example, a polypeptide or peptide may be isolated from a naturally occurring source. Alternatively, a nucleic acid encoding the polypeptide or peptide may be expressed in vitro, such as in an E. coli expression system, as is well known in the art, and isolated in amounts useful for therapeutic compositions. Such methods are discussed in detail elsewhere in this specification.

### 3. Methods of Administration, Formulations and Dosage

Methods of administering a disclosed therapeutic include, but are not limited to, intrathecal, intradermal, intramuscular, intraperitoneal (ip), intravenous (iv), subcutaneous, intranasal, epidural, intradural, intracranial, intraventricular, and oral routes. A therapeutic may be administered by any convenient route, including, for example, infusion or bolus injection, topical, absorption through epithelial or mucocutaneous linings (for example, oral mucosa, rectal and intestinal mucosa, vaginal mucosa and the like), ophthalmic, nasal, and transdermal, and may be administered together with other biologically active agents. Administration can be systemic or local. In some instances, injection may be facilitated by a catheter, for example, attached to a reservoir.

In a specific embodiment, it may be desirable to administer a pharmaceutical composition locally to the area in need of treatment. This may be achieved by, for example, and not by way of limitation, local or regional infusion or perfusion during or following surgery, topical application (for example, wound dressing), injection, catheter, suppository, or implant (for example, implants formed from porous, non-porous, or gelatinous materials, including membranes, such as sialastic membranes or fibers), and the like. In one embodiment, a pump may be used (see, e.g., Langer Science 249, 1527, 1990; Sefton Crit. Rev. Biomed. Eng. 14: 201, 1987; Buchwald et al., Surgery 88: 507, 1980; Saudek et al., N. Engl. J. Med. 321: 574, 1989). In one specific example, administration is achieved by intravenous, intradural, intracranial, intrathecal, or epidural infusion of a therapeutic using a transplanted minipump. Such minipump may be transplanted in any location that permits effective delivery of the therapeutic agent to the target site; for instance, a minipump may be transplanted near the tumor. In another embodiment, administration can be by direct injection at the site (or former site) of a tissue that is to be treated, such as the ovarian tumor site. In another embodiment, a therapeutic is delivered in a vesicle, in particular liposomes (see, e.g., Langer, Science 249, 1527, 1990; Treat et al., in Liposomes in the Therapy of Infectious Disease and Cancer, Lopez-Berestein and Fidler (eds.), Liss, N.Y., pp. 353-365, 1989).

In yet another embodiment, a therapeutic agent can be delivered in a controlled release system. In another embodiment, polymeric materials can be used (see, e.g., Ranger et al., Macromol. Sci. Rev. Macromol. Chem. 23: 61, 1983; Levy et al., Science 228: 190, 1985; During et al., Ann. Neurol. 25: 351, 1989; Howard et al., J. Neurosurg. 71: 105, 1989). Other controlled release systems, such as those discussed in the review by Langer (Science 249: 1527, 1990), can also be used.

The vehicle in which an agent is delivered can include pharmaceutically acceptable compositions known to those with skill in the art. For instance, in some embodiments, therapeutic agents disclosed herein are contained in a pharmaceutically acceptable carrier. The term “pharmaceutically acceptable” means approved by a regulatory agency of the federal or a state government or listed in the U.S. Pharmacopoeia or other generally recognized pharmacopoeia for use in animals, and, more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable, or synthetic origin, such as peanut oil, soybean oil, mineral oil, sesame oil, and the like. Water is an exemplary carrier when the pharmaceutical composition is administered intravenously. Saline solutions, blood plasma medium, aqueous dextrose, and glycerol solutions can also be employed as liquid carriers, particularly for injectable solutions. The medium may also contain conventional pharmaceutical adjunct materials such as for example, pharmaceutically acceptable salts to adjust the osmotic pressure, lipid carriers such as cyclodextrins, proteins such as serum albumin, hydrophilic agents such as methyl cellulose, detergents, buffers, preservatives and the like.

Examples of pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The therapeutic, if desired, can also contain minor amounts of wetting or emulsifying agents, or pH buffering agents. The therapeutic can take the form of solutions, suspensions, emulsion, tablets, pills, capsules, powders, sustained-release formulations, and the like. The therapeutic can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like. A more complete explanation of parenteral pharmaceutical carriers can be found in Remington: The Science and Practice of Pharmacy (19th Edition, 1995) in chapter 95.

Embodiments of other pharmaceutical compositions are prepared with conventional pharmaceutically acceptable counterions, as would be known to those of skill in the art.

Therapeutic preparations will contain a therapeutically effective amount of at least one active ingredient, preferably in purified form, together with a suitable amount of carrier so as to provide proper administration to the patient. The formulation should suit the mode of administration.

Therapeutic agents of this disclosure can be formulated in accordance with routine procedures as a pharmaceutical composition adapted for intravenous administration to human beings. Typically, compositions for intravenous administration are solutions in sterile isotonic aqueous buffer. Where desired, the composition may also include a solubilizing agent and biologically active or inactive compounds (or both), such as antineoplastic agents and conventional nontoxic pharmaceutically acceptable carriers, respectively.

The ingredients in various embodiments are supplied either separately or mixed together in unit dosage form, for example, in solid, semi-solid and liquid dosage forms such as tablets, pills, powders, liquid solutions, or suspensions, or as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to administration.

The amount of the therapeutic that will be effective depends on the nature of the disorder or condition to be treated, as well as the stage of the disorder or condition. Effective amounts can be determined by standard clinical techniques. The precise dose to be employed in the formulation will also depend on the route of administration, and should be decided according to the judgment of the health care practitioner and each patient's circumstances.

The specific dose level and frequency of dosage for any particular subject may be varied and will depend upon a variety of factors, including the activity of the specific compound, the metabolic stability and length of action of that compound, the age, body weight, general health, sex, diet, mode and time of administration, rate of excretion, drug combination, and severity of the condition of the host undergoing therapy.

The therapeutic agents of the present disclosure can be administered at about the same dose throughout a treatment period, in an escalating dose regimen, or in a loading-dose regime (for example, in which the loading dose is about two to five times the maintenance dose). In some embodiments, the dose is varied during the course of a treatment based on the condition of the subject being treated, the severity of the disease or condition, the apparent response to the therapy, and/or other factors as judged by one of ordinary skill in the art. In some examples, long-term treatment with a disclosed therapeutic is contemplated, for instance in order to have sustained decreased expression or activity of a chemotherapy sensitivity-related molecule which is increased in a chemorefractory or chemoresistant ovarian tumor.

In one example, the method includes daily administration of at least 1 μg of the composition to the subject (such as a human subject). For example, a human can be administered at least 1 μg or at least 1 mg of the composition daily, such as 10 μg to 100 μg daily, 100 μg to 1000 μg daily, for example 10 μg daily, 100 μg daily, or 1000 μg daily. In one example, the subject is administered at least 1 μg (such as 1-100 μg) intravenously of the composition including a binding agent that specifically binds to one or more of the disclosed chemotherapy sensitivity-related molecules. In one example, the subject is administered at least 1 mg intramuscularly (for example in an extremity) of such composition. The dosage can be administered in divided doses (such as 2, 3, or 4 divided doses per day), or in a single dosage daily.

In particular examples, the subject is administered the therapeutic composition that includes a binding agent specific for one or more of the disclosed chemotherapy sensitivity-related molecules on a multiple daily dosing schedule, such as at least two consecutive days, 10 consecutive days, and so forth, for example for a period of weeks, months, or years. In one example, the subject is administered the therapeutic composition that a binding agent specific for one or more of the disclosed chemotherapy sensitivity-related molecules daily for a period of at least 30 days, such as at least 2 months, at least 4 months, at least 6 months, at least 12 months, at least 24 months, or at least 36 months.

### Additional Treatments

In particular examples, prior to, during, or following administration of a therapeutic amount of an agent that reduces or inhibits chemoresistance or chemorefraction due to the interaction of a binding agent with one or more of the disclosed chemotherapy sensitivity-related molecules, the subject can receive one or more other therapies. In one example, the subject receives one or more treatments to remove or reduce the tumor prior to administration of a therapeutic amount of a composition including a binding agent specific for one or more of the disclosed chemotherapy sensitivity-related molecules.

Examples of such therapies include, but are not limited to, surgical treatment for removal or reduction of the tumor (such as surgical resection, cryotherapy, or chemoembolization), as well as anti-tumor pharmaceutical treatments which can include radiotherapeutic agents, anti-neoplastic chemotherapeutic agents, antibiotics, alkylating agents and antioxidants, kinase inhibitors, and other agents. Particular examples of additional therapeutic agents that can be used include microtubule binding agents, DNA intercalators or cross-linkers, DNA synthesis inhibitors, DNA and/or RNA transcription inhibitors, antibodies, enzymes, enzyme inhibitors, and gene regulators. These agents (which are administered at a therapeutically effective amount) and treatments can be used alone or in combination. Methods and therapeutic dosages of such agents are known to those skilled in the art, and can be determined by a skilled clinician.

“Microtubule binding agent” refers to an agent that interacts with tubulin to stabilize or destabilize microtubule formation thereby inhibiting cell division. Examples of microtubule binding agents that can be used in conjunction with the disclosed therapy include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used and are known to those of ordinary skill in the art. For example, suitable epothilones and epothilone analogs are described in International Publication No. WO 2004/018478. Taxoids, such as paclitaxel and docetaxel, as well as the analogs of paclitaxel taught by U.S. Pat. Nos. 6,610,860; 5,530,020; and 5,912,264 can be used.

Suitable DNA and/or RNA transcription regulators, including, without limitation, actinomycin D, daunorubicin, doxorubicin and derivatives and analogs thereof also are suitable for use in combination with the disclosed therapies.

DNA intercalators and cross-linking agents that can be administered to a subject include, without limitation, cisplatin, carboplatin, oxaliplatin, mitomycins, such as mitomycin C, bleomycin, chlorambucil, cyclophosphamide and derivatives and analogs thereof.

DNA synthesis inhibitors suitable for use as therapeutic agents include, without limitation, methotrexate, 5-fluoro-5′-deoxyuridine, 5-fluorouracil and analogs thereof.

Examples of suitable enzyme inhibitors include, without limitation, camptothecin, etoposide, formestane, trichostatin and derivatives and analogs thereof.

Suitable compounds that affect gene regulation include agents that result in increased or decreased expression of one or more genes, such as raloxifene, 5-azacytidine, 5-aza-2′-deoxycytidine, tamoxifen, 4-hydroxytamoxifen, mifepristone and derivatives and analogs thereof.

Kinase inhibitors include Gleevac, Iressa, and Tarceva that prevent phosphorylation and activation of growth factors.

Other therapeutic agents, for example anti-tumor agents, that may or may not fall under one or more of the classifications above, also are suitable for administration in combination with the disclosed therapies. By way of example, such agents include adriamycin, apigenin, rapamycin, zebularine, cimetidine, and derivatives and analogs thereof.

In one example, the therapeutic composition (such as one including a binding agent specific for one or more of the disclosed chemotherapy sensitivity-related molecules) is injected into the subject in the presence of an adjuvant. An adjuvant is an agent that when used in combination with an immunogenic agent augments or otherwise alters or modifies a resultant immune response. In some examples, an adjuvant increases the titer of antibodies induced in a subject by the immunogenic agent. In one example, the one or more peptides are administered to the subject as an emulsion with a IFA and sterile water for injection (for example an intravenous or intramuscular injection). Incomplete Freund's Adjuvant (Seppic, Inc.) can be used as the Freund's Incomplete Adjuvant (IFA) (Fairfield, N.J.). In some examples, IFA is provided in 3 ml of a mineral oil solution based on mannide oleate (Montanide ISA-51). At the time of injection, the peptide(s) is mixed with the Montanide ISA.51 and then administered to the subject. Other adjuvants can be used, for example, Freund's complete adjuvant, B30-MDP, LA-15-PH, montanide, saponin, aluminum hydroxide, alum, lipids, keyhole lympet protein, hemocyanin, a mycobacterial antigen, and combinations thereof.

In some examples, the subject receiving the therapeutic peptide composition (such as one including a binding agent specific for one or more of the disclosed chemotherapy sensitivity-related molecules) is also administered interleukin-2 (IL-2), for example via intravenous administration. In particular examples, IL-2 (Chiron Corp., Emeryville, Calif.) is administered at a dose of at least 500,000 IU/kg as an intravenous bolus over a 15 minute period every eight hours beginning on the day after administration of the peptides and continuing for up to 5 days. Doses can be skipped depending on subject tolerance.

In some examples, the disclosed compositions can be co-administered with a fully human antibody to cytotoxic T-lymphocyte antigen-4 (anti-CTLA-4). In some example subjects receive at least 1 mg/kg anti-CTLA-4 (such as 3 mg/kg every 3 weeks or 3 mg/kg as the initial dose with subsequent doses reduced to 1 mg/kg every 3 weeks).

In one example, at least a portion of the tumor (such as a metastatic tumor) is surgically removed (for example via cryotherapy), irradiated, chemically treated (for example via chemoembolization) or combinations thereof, prior to administration of the disclosed therapies (such as administration of a binding agent specific for one or more of the disclosed chemotherapy sensitivity-related molecules). For example, a subject having a metastatic tumor can have all or part of the tumor surgically excised prior to administration of the disclosed therapies (such as one including a binding agent specific for one or more of the disclosed chemotherapy sensitivity-related molecules). In an example, one or more chemotherapeutic agents is administered following treatment with a binding agent specific for one or more of the disclosed chemotherapy sensitivity-related molecules. In another particular example, the subject has a metastatic tumor and is administered radiation therapy, chemoembolization therapy, or both concurrently with the administration of the disclosed therapies (such as one including a binding agent specific for one or more of the disclosed chemotherapy sensitivity-related molecules).

### Generation and Administration of siRNA

In an example, certain inhibitors provided by this disclosure are species of siRNAs. One of ordinary skill in the art can readily generate siRNAs which specifically bind to one or more of the disclosed chemotherapy sensitivity-related molecules that are upregulated in chemorefractory or chemoresistant ovarian cancers. In an example, commercially available kits, such as siRNA molecule synthesizing kits from PROMEGA® (Madison, Wis.) or AMBION® (Austin, Tex.) may be used to synthesize siRNA molecules. In another example, siRNAs are obtained from commercial sources, such as from QIAGEN® Inc (Germantown, Md.), INVITROGEN® (Carlsbad, Calif.), AMBION (Austin, Tex.), DHARMACON® (Lafayette, Co.) or OPENBIOSYSTEMS® (Huntsville, Ala.).

In certain examples, expression vectors are employed to express the at least one siRNA molecule. For example, an expression vector can include a nucleic acid sequence encoding at least one siRNA molecule corresponding to at least one of the disclosed chemotherapy sensitivity-related molecules listed in Tables 1 and 5 that are upregulated in chemorefractory or chemoresistant ovarian cancers. In a particular example, the vector contains a sequence(s) encoding both strands of a siRNA molecule comprising a duplex. In another example, the vector also contains sequence(s) encoding a single nucleic acid molecule that is self-complementary and thus forms a siRNA molecule. Non-limiting examples of such expression vectors are described in Paul et al., Nature Biotechnology 19:505, 2002; Miyagishi and Taira, Nature Biotechnology 19:497, 2002; Lee et al., Nature Biotechnology 19:500, 2002; and Novina et al., Nature Medicine, online publication Jun. 3, 2003.

In other examples, siRNA molecules include a delivery vehicle, including inter alia liposomes, for administration to a subject, carriers and diluents and their salts, and can be present in pharmaceutical compositions. Nucleic acid molecules can be administered to cells by a variety of methods known to those of skill in the art, including, but not restricted to, encapsulation in liposomes, by iontophoresis, or by incorporation into other delivery vehicles, such as hydrogels, cyclodextrins, biodegradable nanocapsules, and bioadhesive microspheres, or by proteinaceous vectors (see, for example, O'Hare and Normand, International PCT Publication No. WO 00/53722).

Alternatively, the nucleic acid/vehicle combination can be locally delivered by direct injection or by use of an infusion pump. Direct injection of the nucleic acid molecules of the disclosure, whether subcutaneous, intramuscular, or intradermal, can take place using standard needle and syringe methodologies, or by needle-free technologies such as those described by Barry et al., International PCT Publication No. WO 99/31262. Other delivery routes, but are not limited to, oral delivery (such as in tablet or pill form), intrathecal or intraperitoneal delivery. For example, intraperitoneal delivery can take place by injecting the treatment into the peritoneal cavity of the subject in order to directly deliver the molecules to the tumor site. More detailed descriptions of nucleic acid delivery and administration are provided in Sullivan et al., PCT WO 94/02595, Draper et al., PCT WO93/23569, Beigelman et al., PCT WO99/05094, and Klimuk et al., PCT WO99/04819, all of which are incorporated by reference herein.

Alternatively, certain siRNA molecules can be expressed within cells from eukaryotic promoters. Those skilled in the art will recognize that any nucleic acid can be expressed in eukaryotic cells using the appropriate DNA/RNA vector. The activity of such nucleic acids can be augmented by their release from the primary transcript by an enzymatic nucleic acid (Draper et al., PCT WO 93/23569, and Sullivan et al., PCT WO 94/02595).

In other examples, siRNA molecules can be expressed from transcription units (see for example, Couture et al., 1996, TIG 12:510) inserted into DNA or RNA vectors. The recombinant vectors can be DNA plasmids or viral vectors. siRNA expressing viral vectors can be constructed based on, for example, but not limited to, adeno-associated virus, retrovirus, adenovirus, lentivirus or alphavirus. In another example, pol III based constructs are used to express nucleic acid molecules of the invention (see for example, Thompson, U.S. Pat. Nos. 5,902,880 and 6,146,886).

The recombinant vectors capable of expressing the siRNA molecules can be delivered as described above, and persist in target cells. Alternatively, viral vectors can be used that provide for transient expression of nucleic acid molecules. Such vectors can be repeatedly administered as necessary. Once expressed, the siRNA molecule interacts with the target mRNA and generates an RNAi response. Delivery of siRNA molecule expressing vectors can be systemic, such as by intravenous or intramuscular administration, by administration to target cells ex-planted from a subject followed by reintroduction into the subject, or by any other means that would allow for introduction into the desired target cell.

### Generation of Antibodies

One of ordinary skill in the art can readily generate antibodies which specifically bind to the disclosed chemotherapy sensitivity-related molecules. These antibodies can be monoclonal or polyclonal. They can be chimeric or humanized. Any functional fragment or derivative of an antibody can be used including Fab, Fab′, Fab2, Fab′2, and single chain variable regions. So long as the fragment or derivative retains specificity of binding for the chemotherapy sensitivity-related molecule it can be used in the methods provided herein. Antibodies can be tested for specificity of binding by comparing binding to appropriate antigen to binding to irrelevant antigen or antigen mixture under a given set of conditions. If the antibody binds to appropriate antigen at least 2, at least 5, at least 7 or 10 times more than to irrelevant antigen or antigen mixture, then it is considered to be specific.

In an example, monoclonal antibodies are generated to the chemotherapy sensitivity-related molecules disclosed in Tables 1 and 5. These monoclonal antibodies each include a variable heavy (V_{H}) and a variable light (V_{L}) chain and specifically bind to the specific chemotherapy sensitivity-related molecules. For example, the antibody can bind the specific chemotherapy sensitivity-related molecules with an affinity constant of at least 10⁶ M⁻¹, such as at least 10⁷ M⁻¹, at least 10⁸ M⁻¹, at least 5×10⁸ M⁻¹, or at least 10⁹ M⁻¹.

The specific antibodies can include a V_{L} polypeptide having amino acid sequences of the complementarity determining regions (CDRs) that are at least about 90% identical, such as at least about 95%, at least about 98%, or at least about 99% identical to the amino acid sequences of the specific chemotherapy sensitivity-related molecules and a V_{H} polypeptide having amino acid sequences of the CDRs that are at least about 90% identical, such as at least about 95%, at least about 98%, or at least about 99% identical to the amino acid sequences of the specific chemotherapy sensitivity-related molecules.

In one example, the sequence of the specificity determining regions of each CDR is determined. Residues that are outside the CDR (non-ligand contacting sites) are substituted. For example, in any of the CDR sequences, at most one, two or three amino acids can be substituted. The production of chimeric antibodies, which include a framework region from one antibody and the CDRs from a different antibody, is well known in the art. For example, humanized antibodies can be routinely produced. The antibody or antibody fragment can be a humanized immunoglobulin having CDRs from a donor monoclonal antibody that binds one of the disclosed chemotherapy sensitivity-related molecules and immunoglobulin and heavy and light chain variable region frameworks from human acceptor immunoglobulin heavy and light chain frameworks. Generally, the humanized immunoglobulin specifically binds to one of the disclosed chemotherapy sensitivity-related molecules with an affinity constant of at least 10⁷ M⁻¹, such as at least 10⁸ M⁻¹ at least 5×10⁸ M₋₁ or at least 10⁹ M⁻¹.

In another example, human monoclonal antibodies to the disclosed chemotherapy sensitivity-related molecules in Tables 1 and 5 are produced. Human monoclonal antibodies can be produced by transferring donor complementarity determining regions (CDRs) from heavy and light variable chains of the donor mouse immunoglobulin into a human variable domain, and then substituting human residues in the framework regions when required to retain affinity. The use of antibody components derived from humanized monoclonal antibodies obviates potential problems associated with the immunogenicity of the constant regions of the donor antibody. For example, when mouse monoclonal antibodies are used therapeutically, the development of human anti-mouse antibodies (HAMA) leads to clearance of the murine monoclonal antibodies and other possible adverse events. Chimeric monoclonal antibodies, with human constant regions, humanized monoclonal antibodies, retaining only murine CDRs, and “fully human” monoclonal antibodies made from phage libraries or transgenic mice have all been used to reduce or eliminate the murine content of therapeutic monoclonal antibodies.

Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Natl. Acad. Sci. U.S.A. 89:4285, 1992; Sandhu, Crit. Rev. Biotech. 12:437, 1992; and Singer et al., J. Immunol. 150:2844, 1993. The antibody may be of any isotype, but in several embodiments the antibody is an IgG, including but not limited to, IgG₁, IgG₂, IgG₃ and IgG₄.

In one example, the sequence of the humanized immunoglobulin heavy chain variable region framework can be at least about 65% identical to the sequence of the donor immunoglobulin heavy chain variable region framework. Thus, the sequence of the humanized immunoglobulin heavy chain variable region framework can be at least about 75%, at least about 85%, at least about 99% or at least about 95%, identical to the sequence of the donor immunoglobulin heavy chain variable region framework. Human framework regions, and mutations that can be made in a humanized antibody framework regions, are known in the art (see, for example, in U.S. Pat. No. 5,585,089, which is incorporated herein by reference).

Antibodies, such as murine monoclonal antibodies, chimeric antibodies, and humanized antibodies, include full length molecules as well as fragments thereof, such as Fab, F(ab′)₂, and Fv, which include a heavy chain and light chain variable region and are capable of binding the epitopic determinant. These antibody fragments retain some ability to selectively bind with their antigen or receptor. These fragments include: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)₂ is a dimer of two Fab′ fragments held together by two disulfide bonds; (4) Fv, a genetically engineered fragment containing the variable region of the light chain and the variable region of the heavy chain expressed as two chains; and (5) Single chain antibody (such as scFv), defined as a genetically engineered molecule containing the variable region of the light chain, the variable region of the heavy chain, linked by a suitable polypeptide linker as a genetically fused single chain molecule. Methods of making these fragments are known in the art (see, for example, Harlow and Lane, Antibodies: A Laboratory Manual, Cold Spring Harbor Laboratory, New York, 1988). Fv antibodies are typically about 25 kDa and contain a complete antigen-binding site with three CDRs per each heavy chain and each light chain. To produce these antibodies, the V_{H} and the V_{L} can be expressed from two individual nucleic acid constructs in a host cell. If the V_{H} and the V_{L} are expressed non-contiguously, the chains of the Fv antibody are typically held together by noncovalent interactions. However, these chains tend to dissociate upon dilution, so methods have been developed to crosslink the chains through glutaraldehyde, intermolecular disulfides, or a peptide linker. Thus, in one example, the Fv can be a disulfide stabilized Fv (dsFv), wherein the heavy chain variable region and the light chain variable region are chemically linked by disulfide bonds.

In an additional example, the Fv fragments include V_{H} and V_{L} chains connected by a peptide linker. These single-chain antigen binding proteins (scFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing scFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; and Sandhu, supra).

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

One of skill will realize that conservative variants of the antibodies can be produced. Such conservative variants employed in antibody fragments, such as dsFv fragments or in scFv fragments, will retain critical amino acid residues necessary for correct folding and stabilizing between the V_{H} and the V_{L} regions, and will retain the charge characteristics of the residues in order to preserve the low pI and low toxicity of the molecules. Amino acid substitutions (such as at most one, at most two, at most three, at most four, or at most five amino acid substitutions) can be made in the V_{H} and the V_{L} regions to increase yield. Conservative amino acid substitution tables providing functionally similar amino acids are well known to one of ordinary skill in the art. The following six groups are examples of amino acids that are considered to be conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

### Kits

Provided by this disclosure are kits that can be used to diagnose, prognose, or treat ovarian cancer that differentially expresses one or more of the disclosed chemotherapy-sensitivity related molecules. The disclosed kits can include instructional materials disclosing means of use of the compositions in the kit. The instructional materials can be written, in an electronic form (such as a computer diskette or compact disk) or can be visual (such as video files).

Kits are provided that can be used in the therapies and diagnostic assays disclosed herein. For example, kits can include one or more of the disclosed therapeutic compositions (such as a composition including one or more of the siRNAs directed to one or more of the chemotherapy sensitivity-related molecules upregulated in chemorefractory or chemoresistant ovarian cancer), one or more of the disclosed gene profile signatures, or combinations thereof. One skilled in the art will appreciate that the kits can include other agents to facilitate the particular application for which the kit is designed.

In one example, a kit is provided for treating an ovarian cancer that is chemoresistant or chemorefractory. For example, such kits can include one or more of the disclosed therapeutic compositions (such as a composition including a siRNA or antibody specific for one or more of the chemotherapy sensitivity-related molecules that are upregulated in chemorefractory or chemoresistant ovarian cancers).

In some example, a kit is provided for detecting one or more of the disclosed chemosensitivity-related molecules in a biological sample, such as serum. Kits for detecting chemosensitivity-related molecules can include one or more probes that specifically bind to the molecules. In an example, a kit includes an array with one or more chemorefractory or chemoresistant molecules and controls, such as positive and negative controls. In other examples, kits include antibodies that specifically bind to one of the chemoresistant or chemorefractory molecules disclosed herein. In some examples, the antibody is labeled (for example, with a fluorescent, radioactive, or an enzymatic label). Such a diagnostic kit can additionally contain means of detecting a label (such as enzyme substrates for enzymatic labels, filter sets to detect fluorescent labels, appropriate secondary labels such as a secondary antibody, or the like), as well as buffers and other reagents routinely used for the practice of a particular diagnostic method.

The disclosure is further illustrated by the following non-limiting Examples.

### EXAMPLE 1

### Materials and Methods

Tissue specimens. Tumor specimens were obtained from 52 previously untreated ovarian cancer subjects hospitalized at the Brigham and Women's hospital between 1990 and 2000. All of the specimens were obtained from primary ovarian tumors. Classification was determined according to the International Federation of Gynecology and Obstetrics (FIGO) standards.

Microdissection and RNA isolation. Frozen sections (7 μm) were affixed to FRAME Slides (Leica, Germany), fixed in 70% alcohol for 30 seconds, stained by 1% methylgreen, washed in water and air-dried. Microdissection was performed using a laser microdissecting microscope (Leica, Germany). Approximately 5,000 tumor cells were dissected for each case. RNA was isolated immediately in 65 μl RLT (Guanidine Isothiocyanate) lysis buffer and was extracted and purified using the RNEASY® Micro Kit according to the manufacturer's protocol (QIAGEN®, Valencia, Calif.). Total RNA was subsequently isolated using the RNEASY® Micro Kit (QIAGEN®, Valencia, Calif.). All purified total RNA specimens were quantified and checked for quality with a Bioanalyzer 2100 system (AGILENT®, Palo Alto, Calif.) before further manipulation.

Total RNA amplification for AFFYMETRIX® GENECHIP® hybridization and image acquisition. To generate sufficient labeled cRNA for microarray analysis from 25 ng of total RNA, two rounds of amplification were necessary. Use of the two-cycle AFFYMETRIX® amplification method has been successfully applied to the linear amplification of small ovarian biopsies. As compared to one-cycle amplification, the two-cycle protocol yielded high quality labeled cRNA product. In addition, the hybridization controls and percent present calls compared favorably between the two protocols suggesting that the bias, if any, introduced during linear amplification did not dramatically affect the hybridization and subsequent data analysis (Kitahara et al., Cancer Research 61: 3544-3549, 2001). For first round synthesis of double stranded cDNA 25 ng of total RNA was reverse transcribed using the Two-Cycle cDNA Synthesis Kit (AFFYMETRIX®, Santa Clara, Calif.) and oligo-dT24-T7 (SEQ ID NO. 1: 5′-GGC CAG TGA ATT GTA ATA CGA CTC ACT ATA GGG AGG CGG-3′) primer according to the manufacturer's instructions followed by amplification with the MEGAscript® T7 Kit (AMBION®, Inc., Austin, Tex.). After clean-up of the cRNA with a GENECHIP® Sample Cleanup Module IVT column (AFFYMETRIX®, Santa Clara, Calif.), second round double stranded cDNA was amplified using the IVT Labeling Kit (AFFYMETRIX®, Santa Clara, Calif.). A 15.0 μg aliquot of labeled product was fragmented by heat and ion-mediated hydrolysis at 94° C. for 35 minutes in 24 μl H₂O and 6 μl of 5× Fragmentation Buffer (AFFYMETRIX®, Santa Clara, Calif.). The fragmented cRNA was hybridized for 16 hr at 45° C. in a Hybridization Oven 640 to a U133 Plus 2.0 oligonucleotide array (AFFYMETRIX®, Santa Clara, Calif.).

Washing and staining of the arrays with phycoerythrin-conjugated streptavidin (Molecular Probes, Eugene, Oreg.) was completed in a Fluidics Station 450 (AFFYMETRIX®, Santa Clara, Calif.). The arrays were then scanned using a confocal laser GENECHIP® Scanner 3000 and GENECHIP® Operating Software (AFFYMETRIX®, Santa Clara, Calif.).

Array Analysis. Data normalization, gene filtering and class prediction analysis were done with BRB-Array Tools Version 3.5.0-Beta_{—}2 (developed by Dr. Richard Simon and Amy Peng Lam of the Biometric Research Branch of National Cancer Institute; available on the world wide web at the National Cancer Institute website). The Robust multiple-array average (RMA) method was used to normalize the array data (Irizarry et al., Biostatistics 4: 249-264, 2003). The RMA method is a three step approach that uses background correction of the PM data (Perfect Match), then applies a quantile normalization and finally summarizes the probe set information by using Tukey's median polish algorithm. Each PM data was log₂-transformed.

Gene filtering criteria was established by excluding from the analysis, genes showing minimal variation (below 50^{th} percentile) across the set of arrays, or found to be absent in more than 50% of the arrays. Class prediction was done using the Compound Covariate Predictor (Radmacher et al., J. Computational Biol. 9: 505-511, 2002), Diagonal Linear Discriminant Analysis (Dudoit et al., J. Amer. Statistical Ass. 97: 77-87, 2002), Nearest Neighbor Classification (Dudoit et al., J. Amer. Statistical Ass. 97: 77-87, 2002), and Support Vector Machines with linear kernel (Ramaswamy et al., Proc. Nat. Acad. Sci. USA 98: 15149-54, 2001) tools available as part of the BRB Array Tools software. The prediction algorithms incorporated genes that were differentially expressed among genes at the 0.001 significance level as assessed by the random variance t-test (Wright and Simon, Bioinformatics 19: 2448-2455, 2003). The prediction error of each model was determined using leave-one-out cross-validation (LOOCV) (Simon et al., J. Nat. Cancer Institute 95: 14-18, 2003). For each LOOCV training set, the entire model building process was repeated, including the gene selection process. It was also determined if the cross-validated error rate estimate for a model was significantly less than one would expect from random prediction. The class labels were randomly permuted and the entire LOOCV process was repeated. The significance level is the proportion of the random permutations that gave a cross-validated error rate no greater than the cross-validated error rate obtained with the real data. One thousand random permutations were used.

qRT-PCR. RNA from ovarian tumors analyzed by microarrays were used to validate the expression of select genes from each predictive gene signature lists. Fifty nanograms of amplified RNA were used as template to perform one-step RT-PCR (INVITROGEN®, Carlsbad, Calif.). Real-time PCR was done according to the recommendations of the manufacturer on a BIORAD® iCyler System (BIORAD®). Relative expression levels of each gene were obtained by normalization to the expression levels of three housekeeping genes (Cyclo, GusB, Gapdh). Log₂ expression values were used for correlation analyses with microarray signal intensities. Pearsons' and Spearmans' rank correlation was performed using GraphPad PRISM® 4.02 (GraphPad Software Inc., San Diego, Calif.).

Cell culture and RNA interference. Ovarian cancer cell lines were routinely maintained in medium supplemented with 10% fetal bovine serum and 2 mM L-glutamine. SKOV3 and OVCAR3 cell lines were maintained in RPMI-1640 medium, OVCA420 and OVCA432 were maintained in 105/199 medium and OVCA420 and CAOV3 cells were maintained in DMEM medium. For 3-(4,5-dimethylthiazol-2-yl)-5-(3-carboxymethoxyphenyl)-2-(4-sulfophenyl)-2H-tetrazolium (MTS) assays, transfections mediated by cationic lipid were performed in 96-well plates. Cells were seeded on a complex of the appropriate siRNA (QIAGEN® Inc., Germantown, Md.) and Oligofectamine (Invitrogen, Carlsbad, Calif.) in unsupplemented growth medium. Final amounts in each well were 50 nM siRNA, 0.5 ul Oligofectamine, and 3000 cells in 100 μL medium. The siRNA target sequences of the synthetic siRNAs were designed against the reference RNA sequence.

siRNA molecules. The REV3L target sequences of the synthetic siRNAs were designed against NM_{—}002912 which is expressly incorporated by reference in its entirety. The siREV3L.1 sequence (Qiagen cat #SI00045626) consisted of sense r(GGAUGUAGUCAAACUGCAA)dTdT (SEQ ID NO:2) and antisense r(UUGCAGUUUGACUACAUCC)dAdG (SEQ ID NO:3), designed against the target CGGGATGTAGTCAAACTGCAA (exon 18; SEQ ID NO:4). The siREV3L.2 sequence (Qiagen cat #SI00045633) consisted of sense r(CACUGGAAUUAAUGCACAA)dTdT (SEQ ID NO:5) and antisense r(UUGUGCAUUAAUUCCAGUG)dTdG (SEQ ID NO:6), designed against the target CCCACTGGAATTAATGCACAA (exon 17; SEQ ID NO:7). The POLH target sequences of the synthetic siRNAs were designed against NM_{—}006502 which is expressly incorporated by reference in its entirety. The siPOLH.2 sequence (Qiagen cat #SI00089012) consisted of sense r(CCAUUUAGGUGCUGAGUUA)dTdT (SEQ ID NO:8) and antisense r(UAACUCAGCACCUAAAUGG)dAdG (SEQ ID NO:9), designed against the target ATCCATTTAGGTGCTGAGTTA (exon 10; SEQ ID NO:10). The siPOLH.5 sequence (Qiagen cat #SI02663619) consisted of sense r(GGUUGUGAGCAUUCGUGUA)dTdT (SEQ ID NO:11) and antisense r(UACACGAAUGCUCACAACC)dTdG (SEQ ID NO:12), designed against the target CTGGTTGTGAGCATTCGTGTA (exon 11; SEQ ID NO:13). The negative control (siNeg) sequence consisted of r(UUCUCCGAACGUGUCACGU)dTdT (SEQ ID NO:14) and r(ACGUGACACGUUCGGAGAA)dTdT (SEQ ID NO:15) strands (Qiagen Inc., Germantown Md.).

MTS proliferation assay. Cell line sensitivity to chemotherapeutic reagents such as cisplatin or taxol was determined by measuring formazan production from MTS (PROMEGA®, Madison, Wis.), with drug concentrations tested in octuplicates in each experiment. For example, serial dilutions of cisplatin or taxol were made shortly before addition to cells. At 48 hours after transfection and seeding, cells were washed by aspiration of the supernatant, and 150 uL of drug-containing medium is added. Another 48 hours later, the drug solution was aspirated and 120 uL MTS-containing medium is added according to the manufacturer's protocol (PROMEGA® #G3580, Madison, Wis.). The plates were incubated at 37° C. and read at 490 nm after 3 hours. Using GraphPad PRISM® 4.02 (GraphPad Software Inc., San Diego, Calif.), the drug concentrations were log-transformed and nonlinear regression is performed on the A₄₉₀ data using the sigmoidal dose response model with variable slope. Mean EC₅₀ values, standard errors, and 95% confidence intervals were determined from the logistic fits.

### EXAMPLE 2

### Development of Chemorefractory Gene Signature

This example describes methods used to identify 105 chemorefractory specific molecules that can be used to predict chemoresponsiveness, such as chemorefraction, in subjects with ovarian cancer.

The training set to develop the predictive refractory to chemotherapy gene signature (refractory gene list) included 12 subject samples whose tumors were refractory to chemotherapy and 13 subject samples whose tumors were sensitive to chemotherapy. The list was refined to include only genes used in all LOOCV iterations. This refinement yielded a 105-gene signature list as illustrated in Table 1. The function and/or location of the respective molecules are provided in Table 2. Genes with a positive t-statistical value are up-regulated in chemorefractory ovarian tumors and genes with a negative t-statistical value are down-regulated.

TABLE 1
Chemorefractory gene signature profile.

| AFFYMETRIX ® | t- | Parametric | Fold Change | UniGene | | LocusLink | |
|---|---|---|---|---|---|---|---|
| PROBE ID | value | p-value | in Refractory | ID # | Symbol | ID number | GENE Name |
| 226538_at | 6.54 | 0.0000011 | 2.999 | Hs.432822 | MAN2A1 | 4124 | Mannosidase, alpha, |
| | | | | | | | class 2A, member 1 |
| 205105_at | 6.35 | 0.0000018 | 2.42 | Hs.432822 | MAN2A1 | 4124 | mannosidase, alpha, |
| | | | | | | | class 2A, member 1 |
| 221156_x_at | 5.89 | 0.0000053 | 1.735 | Hs.285051 | CCPG1 | 9236 | cell cycle |
| | | | | | | | progression 1 |
| 238067_at | 5.5 | 0.0000136 | 2.621 | Hs.351798 | FLJ20298 | 54885 | FLJ20298 protein |
| 226977_at | 5.38 | 0.0000183 | 3.949 | Hs.293782 | LOC492311 | 492311 | similar to bovine |
| | | | | | | | IgA regulatory |
| | | | | | | | protein |
| 226689_at | 5.25 | 0.0000253 | 2.011 | Hs.556638 | LOC493856 | 493856 | similar to RIKEN |
| | | | | | | | cDNA 1500009M05 |
| | | | | | | | gene |
| 201307_at | 5.2 | 0.0000286 | 2.094 | Hs.128199 | SEPT11 | 55752 | septin 11 |
| 216074_x_at | 5.09 | 0.0000375 | 2.084 | Hs.484047 | KIBRA | 23286 | KIBRA protein |
| 203501_at | 5.03 | 0.0000433 | 2.228 | Hs.156178 | PGCP | 10404 | plasma glutamate |
| | | | | | | | carboxypeptidase |
| 224576_at | 5.01 | 0.0000454 | 2.467 | Hs.509163 | KIAA1181 | 57222 | endoplasmic |
| | | | | | | | reticulum-golgi |
| | | | | | | | intermediate |
| | | | | | | | compartment 32 |
| | | | | | | | kDa protein |
| 225275_at | 4.91 | 0.0000582 | 3.895 | Hs.482730 | EDIL3 | 10085 | EGF-like repeats |
| | | | | | | | and discoidin I-like |
| | | | | | | | domains 3 |
| 242981_at | 4.82 | 0.0000721 | 2.209 | | | | |
| 214152_at | 4.81 | 0.0000751 | 1.683 | Hs.285051 | CCPG1 | 9236 | cell cycle |
| | | | | | | | progression 1 |
| 229285_at | 4.8 | 0.0000764 | 3.066 | Hs.518545 | RNASEL | 6041 | ribonuclease L (2′,5′- |
| | | | | | | | oligoisoadenylate |
| | | | | | | | synthetase- |
| | | | | | | | dependent) |
| 230031_at | 4.8 | 0.0000769 | 2.18 | Hs.522394 | HSPA5 | 3309 | heat shock 70 kDa |
| | | | | | | | protein 5 (glucose- |
| | | | | | | | regulated protein, |
| | | | | | | | 78 kDa) |
| 238617_at | 4.78 | 0.0000796 | 4.888 | Hs.143134 | | | CDNA FLJ38181 |
| | | | | | | | fis, clone |
| | | | | | | | FCBBF1000125 |
| 213272_s_at | 4.76 | 0.0000839 | 1.598 | Hs.258212 | LOC57146 | 57146 | Promethin |
| 212764_at | 4.76 | 0.0000851 | 2.884 | | | | |
| 235103_at | 4.74 | 0.0000898 | 1.929 | Hs.432822 | MAN2A1 | 4124 | Mannosidase, alpha, |
| | | | | | | | class 2A, member 1 |
| 225453_x_at | −4.71 | 0.0000949 | −1.594896332 | Hs.100043 | LOC115098 | 115098 | Hypothetical protein |
| | | | | | | | BC013949 |
| 227539_at | 4.71 | 0.0000956 | 2.463 | Hs.515018 | GNA13 | 10672 | Guanine nucleotide |
| | | | | | | | binding protein (G |
| | | | | | | | protein), alpha 13 |
| 233852_at | 4.7 | 0.0000985 | 1.643 | Hs.439153 | POLH | 5429 | Polymerase (DNA |
| | | | | | | | directed), eta |
| 244749_at | 4.69 | 0.0001008 | 2.037 | Hs.44698 | | | CDNA FLJ42484 |
| | | | | | | | fis, clone |
| | | | | | | | BRACE2032182 |
| 203619_s_at | −4.67 | 0.0001061 | −1.295336788 | Hs.182859 | FAIM2 | 23017 | Fas apoptotic |
| | | | | | | | inhibitory molecule 2 |
| 225171_at | 4.65 | 0.0001106 | 2.825 | Hs.486458 | ARHGAP18 | 93663 | Rho GTPase |
| | | | | | | | activating protein 18 |
| 201506_at | 4.62 | 0.0001204 | 2.474 | Hs.369397 | TGFBI | 7045 | transforming growth |
| | | | | | | | factor, beta-induced, |
| | | | | | | | 68 kDa |
| 223512_at | 4.59 | 0.0001282 | 1.769 | Hs.279582 | SARA2 | 51128 | SAR1a gene |
| | | | | | | | homolog 2 (S. |
| | | | | | | | cerevisiae) |
| 201924_at | 4.54 | 0.0001481 | 2.875 | Hs.480190 | AFF1 | 4299 | AF4/FMR2 family, |
| | | | | | | | member 1 |
| 201215_at | 4.53 | 0.0001501 | 4.923 | Hs.496622 | PLS3 | 5358 | plastin 3 (T isoform) |
| 238034_at | 4.51 | 0.0001577 | 2.058 | Hs.529890 | CANX | 821 | Calnexin |
| 206628_at | −4.5 | 0.0001627 | −1.904761905 | Hs.1964 | SLC5A1 | 6523 | solute carrier family |
| | | | | | | | 5 (sodium/glucose |
| | | | | | | | cotransporter), |
| | | | | | | | member 1 |
| 212193_s_at | 4.46 | 0.0001774 | 2.075 | Hs.292078 | LARP1 | 23367 | La ribonucleoprotein |
| | | | | | | | domain family, |
| | | | | | | | member 1 |
| 225823_at | 4.46 | 0.0001782 | 2.234 | Hs.356626 | QIL1 | 125988 | QIL1 protein |
| 211980_at | 4.46 | 0.0001784 | 3.489 | Hs.17441 | COL4A1 | 1282 | collagen, type IV, |
| | | | | | | | alpha 1 |
| 201061_s_at | 4.45 | 0.0001816 | 2.855 | Hs.253903 | STOM | 2040 | Stomatin |
| 213085_s_at | 4.45 | 0.0001854 | 2.933 | Hs.484047 | KIBRA | 23286 | KIBRA protein |
| 1558487_a_at | 4.44 | 0.0001864 | 2.5 | Hs.510745 | TMED4 | 222068 | transmembrane |
| | | | | | | | emp24 protein |
| | | | | | | | transport domain |
| | | | | | | | containing 4 |
| 227761_at | 4.44 | 0.0001886 | 2.621 | Hs.21213 | MY05A | 4644 | myosin VA (heavy |
| | | | | | | | polypeptide 12, |
| | | | | | | | myoxin) |
| 1562488_at | −4.43 | 0.0001948 | −1.449275362 | Hs.434163 | C18orf30 | 284221 | chromosome 18 |
| | | | | | | | open reading frame |
| | | | | | | | 30 |
| 1554583_a_at | −4.41 | 0.0002005 | −1.47275405 | Hs.549290 | MGC50559 | 254013 | hypothetical protein |
| | | | | | | | MGC50559 |
| 214151_s_at | 4.4 | 0.0002077 | 1.628 | Hs.285051 | CCPG1 | 9236 | cell cycle |
| | | | | | | | progression 1 |
| 209404_s_at | 4.38 | 0.000216 | 2.093 | Hs.508765 | TMED7 | 51014 | transmembrane |
| | | | | | | | emp24 protein |
| | | | | | | | transport domain |
| | | | | | | | containing 7 |
| 205407_at | 4.37 | 0.0002224 | 2.771 | Hs.388918 | RECK | 8434 | reversion-inducing- |
| | | | | | | | cysteine-rich protein |
| | | | | | | | with kazal motifs |
| 201413_at | 4.37 | 0.0002243 | 2.463 | Hs.406861 | HSD17B4 | 3295 | hydroxysteroid (17- |
| | | | | | | | beta) dehydrogenase 4 |
| 230728_at | 4.36 | 0.0002273 | 2.207 | Hs.561710 | | | Transcribed locus |
| 219973_at | 4.36 | 0.0002286 | 1.814 | Hs.22895 | ARSJ | 79642 | arylsulfatase J |
| 235352_at | 4.36 | 0.0002288 | 3 | Hs.13500 | | | CDNA FLJ3 1593 |
| | | | | | | | fis, clone |
| | | | | | | | NT2RI2002481 |
| 1558184_s_at | 4.36 | 0.0002317 | 2.072 | Hs.185796 | ZNF17 | 7565 | zinc finger protein |
| | | | | | | | 17 (HPF3, KOX 10) |
| 1564697_a_at | −4.35 | 0.0002344 | −1.515151515 | Hs.334348 | LOC400752 | 400752 | hypothetical gene |
| | | | | | | | supported by |
| | | | | | | | BC006119 |
| 1560065_at | −4.34 | 0.0002424 | −1.5625 | Hs.396644 | PAIP2 | 51247 | poly(A) binding |
| | | | | | | | protein interacting |
| | | | | | | | protein 2 |
| 238276_at | −4.33 | 0.000245 | −1.589825119 | Hs.4859 | CCNL1 | 57018 | Cyclin L1 |
| 203325_s_at | 4.33 | 0.0002496 | 1.971 | Hs.210283 | COL5A1 | 1289 | collagen, type V, |
| | | | | | | | alpha 1 |
| 203823_at | 4.32 | 0.0002532 | 1.631 | Hs.494875 | RGS3 | 5998 | regulator of G- |
| | | | | | | | protein signalling 3 |
| 209304_x_at | 4.32 | 0.0002564 | 1.926 | Hs.110571 | GADD45B | 4616 | growth arrest and |
| | | | | | | | DNA-damage- |
| | | | | | | | inducible, beta |
| 204995_at | 4.31 | 0.0002583 | 1.796 | Hs.500015 | CDK5R1 | 8851 | cyclin-dependent |
| | | | | | | | kinase 5, regulatory |
| | | | | | | | subunit 1 (p35) |
| 227221_at | 4.3 | 0.0002635 | 1.64 | Hs.371609 | | | CDNA FLJ31683 |
| | | | | | | | fis, clone |
| | | | | | | | NT2RI2005353 |
| 212833_at | 4.29 | 0.0002719 | 2.25 | Hs.75639 | LOC91137 | 91137 | hypothetical protein |
| | | | | | | | BC017169 |
| 201041_s_at | 4.28 | 0.0002791 | 3.537 | Hs.171695 | DUSP1 | 1843 | dual specificity |
| | | | | | | | phosphatase 1 |
| 242773_at | −4.27 | 0.0002878 | −1.742160279 | Hs.1964 | SLC5A1 | 6523 | solute carrier family |
| | | | | | | | 5 (sodium/glucose |
| | | | | | | | cotransporter), |
| | | | | | | | member 1 |
| 210966_x_at | 4.27 | 0.00029 | 1.899 | Hs.292078 | LARP1 | 23367 | La ribonucleoprotein |
| | | | | | | | domain family, |
| | | | | | | | member 1 |
| 226831_at | 4.27 | 0.0002905 | 2.437 | Hs.75639 | LOC91137 | 91137 | Hypothetical protein |
| | | | | | | | BC017169 |
| 1561916_at | −4.26 | 0.0002929 | −1.538461538 | Hs.371828 | | 402522 | Similar to GA |
| | | | | | | | binding protein |
| | | | | | | | transcription factor, |
| | | | | | | | alpha subunit |
| | | | | | | | (60 kD); GA-binding |
| | | | | | | | protein transcription |
| | | | | | | | factor, alpha subunit |
| | | | | | | | (60 kD); human |
| | | | | | | | nuclear respiratory |
| | | | | | | | factor-2 subunit |
| | | | | | | | alpha |
| 240036_at | −4.26 | 0.0002949 | −1.712328767 | Hs.464184 | SEC14L1 | 6397 | SEC14-like 1 (S. |
| | | | | | | | cerevisiae) |
| 202125_s_at | 4.26 | 0.0002955 | 2.354 | Hs.152774 | ALS2CR3 | 66008 | amyotrophic lateral |
| | | | | | | | sclerosis 2 (juvenile) |
| | | | | | | | chromosome region, |
| | | | | | | | candidate 3 |
| 1556687_a_at | −4.26 | 0.0002957 | −1.647446458 | Hs.534377 | CLDN10 | 9071 | claudin 10 |
| 224928_at | 4.26 | 0.0002974 | 2.521 | Hs.480792 | SET7 | 80854 | SET domain- |
| | | | | | | | containing protein 7 |
| 211569_s_at | 4.25 | 0.0003056 | 2.131 | Hs.438289 | HADHSC | 3033 | L-3-hydroxyacyl- |
| | | | | | | | Coenzyme A |
| | | | | | | | dehydrogenase, |
| | | | | | | | short chain |
| 242277_at | 4.24 | 0.0003059 | 1.914 | Hs.102471 | PHACTR2 | 9749 | Phosphatase and |
| | | | | | | | actin regulator 2 |
| 208070_s_at | 4.24 | 0.000306 | 2.839 | Hs.232021 | REV3L | 5980 | REV3-like, catalytic |
| | | | | | | | subunit of DNA |
| | | | | | | | polymerase zeta |
| | | | | | | | (yeast) |
| 205927_s_at | −4.24 | 0.00031 | −1.85528757 | Hs.1355 | CTSE | 1510 | cathepsin E |
| 242852_at | 4.23 | 0.0003199 | 1.563 | Hs.467627 | LOC285147 | 285147 | hypothetical protein |
| | | | | | | | LOC285147 |
| 207173_x_at | 4.23 | 0.0003211 | 3.602 | Hs.116471 | CDH11 | 1009 | cadherin 11, type 2, |
| | | | | | | | OB-cadherin |
| | | | | | | | (osteoblast) |
| 201159_s_at | −4.21 | 0.0003318 | −1.404494382 | Hs.532790 | NMT1 | 4836 | N- |
| | | | | | | | myristoyltransferase 1 |
| 228336_at | 4.21 | 0.0003354 | 2.083 | Hs.438851 | KIAA1935 | 114825 | KIAA1935 protein |
| 227873_at | 4.2 | 0.0003392 | 1.776 | Hs.106534 | C5orf14 | 79770 | chromosome 5 open |
| | | | | | | | reading frame 14 |
| 220347_at | −4.2 | 0.0003394 | −1.642036125 | Hs.448342 | C17orf31 | 23293 | Chromosome 17 |
| | | | | | | | open reading frame 13 |
| 225725_at | 4.2 | 0.0003407 | 2.482 | Hs.371609 | | | CDNA FLJ31683 |
| | | | | | | | fis, clone |
| | | | | | | | NT2RI2005353 |
| 230398_at | −4.2 | 0.0003408 | −1.47275405 | Hs.438292 | TNS4 | 84951 | tensin 4 |
| 202310_s_at | 4.2 | 0.0003409 | 4.886 | Hs.172928 | COL1A1 | 1277 | collagen, type I, |
| | | | | | | | alpha 1 |
| 214269_at | 4.19 | 0.0003474 | 1.507 | Hs.410970 | FLJ22269 | 84179 | hypothetical protein |
| | | | | | | | FLJ22269 |
| 201438_at | 4.19 | 0.0003518 | 5.083 | Hs.233240 | COL6A3 | 1293 | collagen, type VI, |
| | | | | | | | alpha 3 |
| 1562033_at | −4.18 | 0.0003575 | −1.492537313 | Hs.560280 | | | CDNA clone |
| | | | | | | | IMAGE: 5300069 |
| 202766_s_at | 4.18 | 0.0003631 | 3.833 | Hs.146447 | FBN1 | 2200 | fibrillin 1 (Marfan |
| | | | | | | | syndrome) |
| 228391_at | 4.18 | 0.0003634 | 2.745 | Hs.237642 | CYP4V2 | 285440 | cytochrome |
| | | | | | | | CHEMOTHERAPY |
| | | | | | | | SENSITIVITY- |
| | | | | | | | RELATED |
| | | | | | | | MOLECULE0, |
| | | | | | | | family 4, subfamily |
| | | | | | | | V, polypeptide 2 |
| 212737_at | 4.18 | 0.0003636 | 1.995 | Hs.483873 | GM2A | 2760 | GM2 ganglioside |
| | | | | | | | activator |
| 227413_at | 4.17 | 0.0003641 | 2.773 | Hs.190447 | UBLCP1 | 134510 | ubiquitin-like |
| | | | | | | | domain containing |
| | | | | | | | CTD phosphatase 1 |
| 225016_at | 4.17 | 0.0003675 | 3.57 | Hs.293274 | APCDD1 | 147495 | adenomatosis |
| | | | | | | | polyposis coli down- |
| | | | | | | | regulated 1 |
| 201944_at | 4.17 | 0.0003681 | 2.478 | Hs.69293 | HEXB | 3074 | hexosaminidase B |
| | | | | | | | (beta polypeptide) |
| 1561226_at | −4.16 | 0.0003736 | −1.589825119 | Hs.128375 | LOC401062 | 401062 | hypothetical gene |
| | | | | | | | supported by |
| | | | | | | | AK092973 |
| 225182_at | 4.16 | 0.0003747 | 2.498 | Hs.433668 | TMEM50B | 757 | transmembrane |
| | | | | | | | protein 50B |
| 238604_at | 4.16 | 0.0003795 | 2.663 | Hs.563482 | | | CDNA FLJ25559 |
| | | | | | | | fis, clone JTH02834 |
| 208005_at | −4.15 | 0.0003828 | −1.424501425 | Hs.128002 | NTN1 | 9423 | netrin 1 |
| 233135_at | −4.15 | 0.0003864 | −1.564945227 | Hs.535863 | | | CDNA clone |
| | | | | | | | IMAGE: 4820713 |
| 227947_at | 4.15 | 0.0003878 | 2.307 | Hs.102471 | PHACTR2 | 9749 | phosphatase and |
| | | | | | | | actin regulator 2 |
| 212895_s_at | 4.15 | 0.0003921 | 1.839 | Hs.159306 | ABR | 29 | active BCR-related |
| | | | | | | | gene |
| 230170_at | 4.13 | 0.0004108 | 1.551 | Hs.248156 | OSM | 5008 | oncostatin M |
| 218323_at | 4.12 | 0.0004204 | 2.009 | Hs.462742 | RHOT1 | 55288 | ras homolog gene |
| | | | | | | | family, member T1 |
| 205022_s_at | 4.12 | 0.0004224 | 1.882 | Hs.434286 | CHES1 | 1112 | checkpoint |
| | | | | | | | suppressor 1 |
| 228315_at | 4.11 | 0.0004253 | 2.33 | Hs.371609 | | | CDNA FLJ31683 |
| | | | | | | | fis, clone |
| | | | | | | | NT2RI2005353 |
| 200906_s_at | 4.11 | 0.0004269 | 2.089 | Hs.151220 | KIAA0992 | 23022 | palladin |
| 212798_s_at | 4.11 | 0.000428 | 2.375 | Hs.157378 | ANKMY2 | 57037 | ankyrin repeat and |
| | | | | | | | MYND domain |
| | | | | | | | containing 2 |
| 209348_s_at | 4.11 | 0.0004288 | 2.651 | Hs.134859 | MAF | 4094 | v-maf |
| | | | | | | | musculoaponeurotic |
| | | | | | | | fibrosarcoma |
| | | | | | | | oncogene homolog |
| | | | | | | | (avian) |
| 40420_at | 4.11 | 0.0004309 | 1.59 | Hs.519756 | STK10 | 6793 | Serine/threonine |
| | | | | | | | kinase 10 |
| 221584_s_at | 4.1 | 0.0004363 | 3.064 | Hs.144795 | KCNMA1 | 3778 | potassium large |
| | | | | | | | conductance |
| | | | | | | | calcium-activated |
| | | | | | | | channel, subfamily |
| | | | | | | | M, alpha member 1 |
| 210809_s_at | 4.1 | 0.0004368 | 7.485 | Hs.136348 | POSTN | 10631 | periostin, osteoblast |
| | | | | | | | specific factor |

TABLE 2
Function and/or location of chemorefractory specific molecules.

| | AFFYMETRIX ® | GENE |
|---|---|---|
| LOCATION/FUNCTION | Probe ID | NAME |
| cell fraction | 226538_at | MAN2A1 |
| cell fraction | 205105_at | MAN2A1 |
| cell fraction | 205407_at | RECK |
| cell fraction | 235103_at | MAN2A1 |
| cell fraction | 208005_at | NTN1 |
| Golgi stack | 226538_at | MAN2A1 |
| Golgi stack | 205105_at | MAN2A1 |
| Golgi stack | 224576_at | KIAA1181 |
| Golgi stack | 235103_at | MAN2A1 |
| Golgi stack | 223512_at | SARA2 |
| Golgi apparatus | 226538_at | MAN2A1 |
| Golgi apparatus | 205105_at | MAN2A1 |
| Golgi apparatus | 240036_at | SEC14L1 |
| Golgi apparatus | 224576_at | KIAA1181 |
| Golgi apparatus | 235103_at | MAN2A1 |
| Golgi apparatus | 223512_at | SARA2 |
| signal transducer activity | 225275_at | EDIL3 |
| signal transducer activity | 203823_at | RGS3 |
| signal transducer activity | 201506_at | TGFBI |
| signal transducer activity | 202125_s_at | ALS2CR3 |
| signal transducer activity | 1558487_a_at | TMED4 |
| integral to membrane | 226538_at | MAN2A1 |
| integral to membrane | 205105_at | MAN2A1 |
| integral to membrane | 214269_at | FLJ22269 |
| integral to membrane | 1562488_at | C18orf30 |
| integral to membrane | 209404_s_at | TMED7 |
| integral to membrane | 224576_at | KIAA1181 |
| integral to membrane | 235103_at | MAN2A1 |
| integral to membrane | 212833_at | LOC91137 |
| integral to membrane | 225182_at | TMEM50B |
| integral to membrane | 201061_s_at | STOM |
| integral to membrane | 203619_s_at | FAIM2 |
| integral to membrane | 206628_at | SLC5A1 |
| integral to membrane | 242773_at | SLC5A1 |
| integral to membrane | 207173_x_at | CDH11 |
| integral to membrane | 1558487_a_at | TMED4 |
| integral to membrane | 228391_at | CYP4V2 |
| integral to membrane | 226831_at | LOC91137 |
| intrinsic to membrane | 226538_at | MAN2A1 |
| intrinsic to membrane | 205105_at | MAN2A1 |
| intrinsic to membrane | 214269_at | FLJ22269 |
| intrinsic to membrane | 1562488_at | C18orf30 |
| intrinsic to membrane | 209404_s_at | TMED7 |
| intrinsic to membrane | 224576_at | KIAA1181 |
| intrinsic to membrane | 235103_at | MAN2A1 |
| intrinsic to membrane | 212833_at | LOC91137 |
| intrinsic to membrane | 225182_at | TMEM50B |
| intrinsic to membrane | 201061_s_at | STOM |
| intrinsic to membrane | 203619_s_at | FAIM2 |
| intrinsic to membrane | 206628_at | SLC5A1 |
| intrinsic to membrane | 242773_at | SLC5A1 |
| intrinsic to membrane | 207173_x_at | CDH11 |
| intrinsic to membrane | 1558487_a_at | TMED4 |
| intrinsic to membrane | 228391_at | CYP4V2 |
| intrinsic to membrane | 226831_at | LOC91137 |
| biological_process | 226538_at | MAN2A1 |
| biological_process | 205105_at | MAN2A1 |
| biological_process | 211980_at | COL4A1 |
| biological_process | 201307_at | septin 11 |
| biological_process | 225275_at | EDIL3 |
| biological_process | 201438_at | COL6A3 |
| biological_process | 201413_at | HSD17B4 |
| biological_process | 214269_at | FLJ22269 |
| biological_process | 202766_s_at | FBN1 |
| biological_process | 220347_at | C17orf31 |
| biological_process | 209404_s_at | TMED7 |
| biological_process | 227873_at | C5orf14 |
| biological_process | 201944_at | HEXB |
| biological_process | 240036_at | SEC14L1 |
| biological_process | 218323_at | RHOT1 |
| biological_process | 224576_at | KIAA1181 |
| biological_process | 1560065_at | PAIP2 |
| biological_process | 205407_at | RECK |
| biological_process | 227413_at | UBLCP1 |
| biological_process | 235103_at | MAN2A1 |
| biological_process | 230398_at | TNS4 |
| biological_process | 208005_at | NTN1 |
| biological_process | 210809_s_at | POSTN |
| biological_process | 238034_at | CANX |
| biological_process | 40420_at | STK10 |
| biological_process | 238276_at | CCNL1 |
| biological_process | 201041_s_at | DUSP1 |
| biological_process | 202125_s_at | ALS2CR3 |
| biological_process | 212833_at | LOC91137 |
| biological_process | 212895_s_at | ABR |
| biological_process | 227761_at | MYO5A |
| biological_process | 203501_at | PGCP |
| biological_process | 203619_s_at | FAIM2 |
| biological_process | 229285_at | RNASEL |
| biological_process | 209348_s_at | MAF |
| biological_process | 224928_at | SET7 |
| biological_process | 200906_s_at | KIAA0992 |
| biological_process | 1558487_a_at | TMED4 |
| biological_process | 228391_at | CYP4V2 |
| biological_process | 203325_s_at | COL5A1 |
| biological_process | 226831_at | LOC91137 |
| biological_process | 1558184_s_at | ZNF17 |
| biological_process | 211569_s_at | HADHSC |
| biological_process | 1556687_a_at | CLDN10 |
| biological_process | 219973_at | ARSJ |
| cellular process | 226538_at | MAN2A1 |
| cellular process | 205105_at | MAN2A1 |
| cellular process | 211980_at | COL4A1 |
| cellular process | 201307_at | septin 11 |
| cellular process | 225275_at | EDIL3 |
| cellular process | 201438_at | COL6A3 |
| cellular process | 201413_at | HSD17B4 |
| cellular process | 214269_at | FLJ22269 |
| cellular process | 220347_at | C17orf31 |
| cellular process | 209404_s_at | TMED7 |
| cellular process | 227873_at | C5orf14 |
| cellular process | 201944_at | HEXB |
| cellular process | 240036_at | SEC14L1 |
| cellular process | 218323_at | RHOT1 |
| cellular process | 224576_at | KIAA1181 |
| cellular process | 1560065_at | PAIP2 |
| cellular process | 205407_at | RECK |
| cellular process | 227413_at | UBLCP1 |
| cellular process | 235103_at | MAN2A1 |
| cellular process | 230398_at | TNS4 |
| cellular process | 208005_at | NTN1 |
| cellular process | 210809_s_at | POSTN |
| cellular process | 238034_at | CANX |
| cellular process | 40420_at | STK10 |
| cellular process | 238276_at | CCNL1 |
| cellular process | 201041_s_at | DUSP1 |
| cellular process | 202125_s_at | ALS2CR3 |
| cellular process | 212833_at | LOC91137 |
| cellular process | 212895_s_at | ABR |
| cellular process | 227761_at | MYO5A |
| cellular process | 203501_at | PGCP |
| cellular process | 203619_s_at | FAIM2 |
| cellular process | 229285_at | RNASEL |
| cellular process | 209348_s_at | MAF |
| cellular process | 224928_at | SET7 |
| cellular process | 200906_s_at | KIAA0992 |
| cellular process | 1558487_a_at | TMED4 |
| cellular process | 228391_at | CYP4V2 |
| cellular process | 203325_s_at | COL5A1 |
| cellular process | 226831_at | LOC91137 |
| cellular process | 1558184_s_at | ZNF17 |
| cellular process | 211569_s_at | HADHSC |
| cellular process | 1556687_a_at | CLDN10 |
| membrane | 226538_at | MAN2A1 |
| membrane | 205105_at | MAN2A1 |
| membrane | 203823_at | RGS3 |
| membrane | 214269_at | FLJ22269 |
| membrane | 1562488_at | C18orf30 |
| membrane | 209404_s_at | TMED7 |
| membrane | 240036_at | SEC14L1 |
| membrane | 224576_at | KIAA1181 |
| membrane | 205407_at | RECK |
| membrane | 235103_at | MAN2A1 |
| membrane | 223512_at | SARA2 |
| membrane | 202125_s_at | ALS2CR3 |
| membrane | 212833_at | LOC91137 |
| membrane | 227539_at | GNA13 |
| membrane | 225182_at | TMEM50B |
| membrane | 201061_s_at | STOM |
| membrane | 203619_s_at | FAIM2 |
| membrane | 206628_at | SLC5A1 |
| membrane | 242773_at | SLC5A1 |
| membrane | 207173_x_at | CDH11 |
| membrane | 1558487_a_at | TMED4 |
| membrane | 228391_at | CYP4V2 |
| membrane | 226831_at | LOC91137 |
| molecular_function | 211980_at | COL4A1 |
| molecular_function | 201307_at | septin 11 |
| molecular_function | 225275_at | EDIL3 |
| molecular_function | 203823_at | RGS3 |
| molecular_function | 202310_s_at | COL1A1 |
| molecular_function | 201506_at | TGFBI |
| molecular_function | 214269_at | FLJ22269 |
| molecular_function | 1554583_a_at | MGC50559 |
| molecular_function | 220347_at | C17orf31 |
| molecular_function | 212737_at | GM2A |
| molecular_function | 201215_at | PLS3 |
| molecular_function | 209404_s_at | TMED7 |
| molecular_function | 227873_at | C5orf14 |
| molecular_function | 240036_at | SEC14L1 |
| molecular_function | 221584_s_at | KCNMA1 |
| molecular_function | 201924_at | AFF1 |
| molecular_function | 218323_at | RHOT1 |
| molecular_function | 1560065_at | PAIP2 |
| molecular_function | 208005_at | NTN1 |
| molecular_function | 210809_s_at | POSTN |
| molecular_function | 210966_x_at | LARP1 |
| molecular_function | 212193_s_at | LARP1 |
| molecular_function | 202125_s_at | ALS2CR3 |
| molecular_function | 212833_at | LOC91137 |
| molecular_function | 212895_s_at | ABR |
| molecular_function | 228336_at | KIAA1935 |
| molecular_function | 225171_at | ARHGAP18 |
| molecular_function | 209348_s_at | MAF |
| molecular_function | 207173_x_at | CDH11 |
| molecular_function | 227947_at | PHACTR2 |
| molecular_function | 1558487_a_at | TMED4 |
| molecular_function | 203325_s_at | COL5A1 |
| molecular_function | 226831_at | LOC91137 |
| molecular_function | 211569_s_at | HADHSC |
| molecular_function | 242277_at | PHACTR2 |
| molecular_function | 1556687_a_at | CLDN10 |
| protein binding | 201307_at | septin 11 |
| protein binding | 225275_at | EDIL3 |
| protein binding | 203823_at | RGS3 |
| protein binding | 201506_at | TGFBI |
| protein binding | 201215_at | PLS3 |
| protein binding | 209404_s_at | TMED7 |
| protein binding | 221584_s_at | KCNMA1 |
| protein binding | 1560065_at | PAIP2 |
| protein binding | 210809_s_at | POSTN |
| protein binding | 202125_s_at | ALS2CR3 |
| protein binding | 225171_at | ARHGAP18 |
| protein binding | 207173_x_at | CDH11 |
| protein binding | 227947_at | PHACTR2 |
| protein binding | 1558487_a_at | TMED4 |
| protein binding | 242277_at | PHACTR2 |
| protein binding | 1556687_a_at | CLDN10 |
| binding | 201307_at | septin 11 |
| binding | 225275_at | EDIL3 |
| binding | 203823_at | RGS3 |
| binding | 201506_at | TGFBI |
| binding | 220347_at | C17orf31 |
| binding | 201215_at | PLS3 |
| binding | 209404_s_at | TMED7 |
| binding | 240036_at | SEC14L1 |
| binding | 221584_s_at | KCNMA1 |
| binding | 201924_at | AFF1 |
| binding | 218323_at | RHOT1 |
| binding | 1560065_at | PAIP2 |
| binding | 210809_s_at | POSTN |
| binding | 210966_x_at | LARP1 |
| binding | 212193_s_at | LARP1 |
| binding | 202125_s_at | ALS2CR3 |
| binding | 212833_at | LOC91137 |
| binding | 225171_at | ARHGAP18 |
| binding | 209348_s_at | MAF |
| binding | 207173_x_at | CDH11 |
| binding | 227947_at | PHACTR2 |
| binding | 1558487_a_at | TMED4 |
| binding | 203325_s_at | COL5A1 |
| binding | 226831_at | LOC91137 |
| binding | 242277_at | PHACTR2 |
| binding | 1556687_a_at | CLDN10 |
| cellular_component | 226538_at | MAN2A1 |
| cellular_component | 205105_at | MAN2A1 |
| cellular_component | 201307_at | septin 11 |
| cellular_component | 203823_at | RGS3 |
| cellular_component | 202310_s_at | COL1A1 |
| cellular_component | 201438_at | COL6A3 |
| cellular_component | 201413_at | HSD17B4 |
| cellular_component | 201506_at | TGFBI |
| cellular_component | 214269_at | FLJ22269 |
| cellular_component | 1554583_a_at | MGC50559 |
| cellular_component | 220347_at | C17orf31 |
| cellular_component | 205022_s_at | CHES1 |
| cellular_component | 1562488_at | C18orf30 |
| cellular_component | 212737_at | GM2A |
| cellular_component | 201215_at | PLS3 |
| cellular_component | 209404_s_at | TMED7 |
| cellular_component | 201944_at | HEXB |
| cellular_component | 240036_at | SEC14L1 |
| cellular_component | 230170_at | OSM |
| cellular_component | 233852_at | POLH |
| cellular_component | 201924_at | AFF1 |
| cellular_component | 224576_at | KIAA1181 |
| cellular_component | 1560065_at | PAIP2 |
| cellular_component | 205407_at | RECK |
| cellular_component | 230031_at | HSPA5 |
| cellular_component | 235103_at | MAN2A1 |
| cellular_component | 208005_at | NTN1 |
| cellular_component | 210809_s_at | POSTN |
| cellular_component | 223512_at | SARA2 |
| cellular_component | 238276_at | CCNL1 |
| cellular_component | 204995_at | CDK5R1 |
| cellular_component | 208070_s_at | REV3L |
| cellular_component | 202125_s_at | ALS2CR3 |
| cellular_component | 212833_at | LOC91137 |
| cellular_component | 227539_at | GNA13 |
| cellular_component | 225182_at | TMEM50B |
| cellular_component | 201061_s_at | STOM |
| cellular_component | 203501_at | PGCP |
| cellular_component | 203619_s_at | FAIM2 |
| cellular_component | 209348_s_at | MAF |
| cellular_component | 206628_at | SLC5A1 |
| cellular_component | 224928_at | SET7 |
| cellular_component | 242773_at | SLC5A1 |
| cellular_component | 207173_x_at | CDH11 |
| cellular_component | 200906_s_at | KIAA0992 |
| cellular_component | 1558487_a_at | TMED4 |
| cellular_component | 228391_at | CYP4V2 |
| cellular_component | 205927_s_at | CTSE |
| cellular_component | 226831_at | LOC91137 |
| cellular_component | 1558184_s_at | ZNF17 |
| cellular_component | 211569_s_at | HADHSC |
| physiological process | 226538_at | MAN2A1 |
| physiological process | 205105_at | MAN2A1 |
| physiological process | 211980_at | COL4A1 |
| physiological process | 201307_at | septin 11 |
| physiological process | 201438_at | COL6A3 |
| physiological process | 201413_at | HSD17B4 |
| physiological process | 214269_at | FLJ22269 |
| physiological process | 202766_s_at | FBN1 |
| physiological process | 220347_at | C17orf31 |
| physiological process | 209404_s_at | TMED7 |
| physiological process | 227873_at | C5orf14 |
| physiological process | 201944_at | HEXB |
| physiological process | 240036_at | SEC14L1 |
| physiological process | 224576_at | KIAA1181 |
| physiological process | 1560065_at | PAIP2 |
| physiological process | 205407_at | RECK |
| physiological process | 227413_at | UBLCP1 |
| physiological process | 235103_at | MAN2A1 |
| physiological process | 208005_at | NTN1 |
| physiological process | 238034_at | CANX |
| physiological process | 40420_at | STK10 |
| physiological process | 238276_at | CCNL1 |
| physiological process | 201041_s_at | DUSP1 |
| physiological process | 202125_s_at | ALS2CR3 |
| physiological process | 212833_at | LOC91137 |
| physiological process | 227761_at | MYO5A |
| physiological process | 203501_at | PGCP |
| physiological process | 203619_s_at | FAIM2 |
| physiological process | 229285_at | RNASEL |
| physiological process | 209348_s_at | MAF |
| physiological process | 224928_at | SET7 |
| physiological process | 200906_s_at | KIAA0992 |
| physiological process | 1558487_a_at | TMED4 |
| physiological process | 228391_at | CYP4V2 |
| physiological process | 203325_s_at | COL5A1 |
| physiological process | 226831_at | LOC91137 |
| physiological process | 1558184_s_at | ZNF17 |
| physiological process | 211569_s_at | HADHSC |
| physiological process | 219973_at | ARSJ |
| calcium ion binding | 225275_at | EDIL3 |
| calcium ion binding | 201215_at | PLS3 |
| calcium ion binding | 221584_s_at | KCNMA1 |
| calcium ion binding | 218323_at | RHOT1 |
| calcium ion binding | 207173_x_at | CDH11 |
| ion binding | 225275_at | EDIL3 |
| ion binding | 201215_at | PLS3 |
| ion binding | 221584_s_at | KCNMA1 |
| ion binding | 218323_at | RHOT1 |
| ion binding | 207173_x_at | CDH11 |
| cation binding | 225275_at | EDIL3 |
| cation binding | 201215_at | PLS3 |
| cation binding | 221584_s_at | KCNMA1 |
| cation binding | 218323_at | RHOT1 |
| cation binding | 207173_x_at | CDH11 |
| metal ion binding | 225275_at | EDIL3 |
| metal ion binding | 201215_at | PLS3 |
| metal ion binding | 221584_s_at | KCNMA1 |
| metal ion binding | 218323_at | RHOT1 |
| metal ion binding | 207173_x_at | CDH11 |
| cytoplasm | 226538_at | MAN2A1 |
| cytoplasm | 205105_at | MAN2A1 |
| cytoplasm | 203823_at | RGS3 |
| cytoplasm | 202310_s_at | COL1A1 |
| cytoplasm | 201438_at | COL6A3 |
| cytoplasm | 201413_at | HSD17B4 |
| cytoplasm | 212737_at | GM2A |
| cytoplasm | 209404_s_at | TMED7 |
| cytoplasm | 201944_at | HEXB |
| cytoplasm | 240036_at | SEC14L1 |
| cytoplasm | 224576_at | KIAA1181 |
| cytoplasm | 1560065_at | PAIP2 |
| cytoplasm | 230031_at | HSPA5 |
| cytoplasm | 235103_at | MAN2A1 |
| cytoplasm | 223512_at | SARA2 |
| cytoplasm | 202125_s_at | ALS2CR3 |
| cytoplasm | 203501_at | PGCP |
| cytoplasm | 1558487_a_at | TMED4 |
| cytoplasm | 228391_at | CYP4V2 |
| cytoplasm | 205927_s_at | CTSE |
| cytoplasm | 211569_s_at | HADHSC |
| Golgi apparatus | 226538_at | MAN2A1 |
| Golgi apparatus | 205105_at | MAN2A1 |
| Golgi apparatus | 240036_at | SEC14L1 |
| Golgi apparatus | 224576_at | KIAA1181 |
| Golgi apparatus | 235103_at | MAN2A1 |
| Golgi apparatus | 223512_at | SARA2 |
| cellular physiological process | 226538_at | MAN2A1 |
| cellular physiological process | 205105_at | MAN2A1 |
| cellular physiological process | 211980_at | COL4A1 |
| cellular physiological process | 201307_at | septin 11 |
| cellular physiological process | 201438_at | COL6A3 |
| cellular physiological process | 201413_at | HSD17B4 |
| cellular physiological process | 214269_at | FLJ22269 |
| cellular physiological process | 220347_at | C17orf31 |
| cellular physiological process | 209404_s_at | TMED7 |
| cellular physiological process | 227873_at | C5orf14 |
| cellular physiological process | 201944_at | HEXB |
| cellular physiological process | 240036_at | SEC14L1 |
| cellular physiological process | 224576_at | KIAA1181 |
| cellular physiological process | 1560065_at | PAIP2 |
| cellular physiological process | 205407_at | RECK |
| cellular physiological process | 227413_at | UBLCP1 |
| cellular physiological process | 235103_at | MAN2A1 |
| cellular physiological process | 208005_at | NTN1 |
| cellular physiological process | 238034_at | CANX |
| cellular physiological process | 40420_at | STK10 |
| cellular physiological process | 238276_at | CCNL1 |
| cellular physiological process | 201041_s_at | DUSP1 |
| cellular physiological process | 202125_s_at | ALS2CR3 |
| cellular physiological process | 212833_at | LOC91137 |
| cellular physiological process | 227761_at | MYO5A |
| cellular physiological process | 203501_at | PGCP |
| cellular physiological process | 203619_s_at | FAIM2 |
| cellular physiological process | 229285_at | RNASEL |
| cellular physiological process | 209348_s_at | MAF |
| cellular physiological process | 224928_at | SET7 |
| cellular physiological process | 200906_s_at | KIAA0992 |
| cellular physiological process | 1558487_a_at | TMED4 |
| cellular physiological process | 228391_at | CYP4V2 |
| cellular physiological process | 203325_s_at | COL5A1 |
| cellular physiological process | 226831_at | LOC91137 |
| cellular physiological process | 1558184_s_at | ZNF17 |
| cellular physiological process | 211569_s_at | HADHSC |
| cell | 226538_at | MAN2A1 |
| cell | 205105_at | MAN2A1 |
| cell | 201307_at | septin 11 |
| cell | 203823_at | RGS3 |
| cell | 202310_s_at | COL1A1 |
| cell | 201438_at | COL6A3 |
| cell | 201413_at | HSD17B4 |
| cell | 214269_at | FLJ22269 |
| cell | 1554583_a_at | MGC50559 |
| cell | 220347_at | C17orf31 |
| cell | 205022_s_at | CHES1 |
| cell | 1562488_at | C18orf30 |
| cell | 212737_at | GM2A |
| cell | 201215_at | PLS3 |
| cell | 209404_s_at | TMED7 |
| cell | 201944_at | HEXB |
| cell | 240036_at | SEC14L1 |
| cell | 233852_at | POLH |
| cell | 201924_at | AFF1 |
| cell | 224576_at | KIAA1181 |
| cell | 1560065_at | PAIP2 |
| cell | 205407_at | RECK |
| cell | 230031_at | HSPA5 |
| cell | 235103_at | MAN2A1 |
| cell | 208005_at | NTN1 |
| cell | 223512_at | SARA2 |
| cell | 238276_at | CCNL1 |
| cell | 204995_at | CDK5R1 |
| cell | 208070_s_at | REV3L |
| cell | 202125_s_at | ALS2CR3 |
| cell | 212833_at | LOC91137 |
| cell | 227539_at | GNA13 |
| cell | 225182_at | TMEM50B |
| cell | 201061_s_at | STOM |
| cell | 203501_at | PGCP |
| cell | 203619_s_at | FAIM2 |
| cell | 209348_s_at | MAF |
| cell | 206628_at | SLC5A1 |
| cell | 224928_at | SET7 |
| cell | 242773_at | SLC5A1 |
| cell | 207173_x_at | CDH11 |
| cell | 200906_s_at | KIAA0992 |
| cell | 1558487_a_at | TMED4 |
| cell | 228391_at | CYP4V2 |
| cell | 205927_s_at | CTSE |
| cell | 226831_at | LOC91137 |
| cell | 1558184_s_at | ZNF17 |
| cell | 211569_s_at | HADHSC |
| Golgi stack | 226538_at | MAN2A1 |
| Golgi stack | 205105_at | MAN2A1 |
| Golgi stack | 224576_at | KIAA1181 |
| Golgi stack | 235103_at | MAN2A1 |
| Golgi stack | 223512_at | SARA2 |
| intracellular | 226538_at | MAN2A1 |
| intracellular | 205105_at | MAN2A1 |
| intracellular | 201307_at | septin 11 |
| intracellular | 203823_at | RGS3 |
| intracellular | 202310_s_at | COL1A1 |
| intracellular | 201438_at | COL6A3 |
| intracellular | 201413_at | HSD17B4 |
| intracellular | 1554583_a_at | MGC50559 |
| intracellular | 220347_at | C17orf31 |
| intracellular | 205022_s_at | CHES1 |
| intracellular | 212737_at | GM2A |
| intracellular | 201215_at | PLS3 |
| intracellular | 209404_s_at | TMED7 |
| intracellular | 201944_at | HEXB |
| intracellular | 240036_at | SEC14L1 |
| intracellular | 233852_at | POLH |
| intracellular | 201924_at | AFF1 |
| intracellular | 224576_at | KIAA1181 |
| intracellular | 1560065_at | PAIP2 |
| intracellular | 230031_at | HSPA5 |
| intracellular | 235103_at | MAN2A1 |
| intracellular | 223512_at | SARA2 |
| intracellular | 238276_at | CCNL1 |
| intracellular | 204995_at | CDK5R1 |
| intracellular | 208070_s_at | REV3L |
| intracellular | 202125_s_at | ALS2CR3 |
| intracellular | 201061_s_at | STOM |
| intracellular | 203501_at | PGCP |
| intracellular | 209348_s_at | MAF |
| intracellular | 224928_at | SET7 |
| intracellular | 200906_s_at | KIAA0992 |
| intracellular | 1558487_a_at | TMED4 |
| intracellular | 228391_at | CYP4V2 |
| intracellular | 205927_s_at | CTSE |
| intracellular | 1558184_s_at | ZNF17 |
| intracellular | 211569_s_at | HADHSC |
| localization | 211980_at | COL4A1 |
| localization | 201438_at | COL6A3 |
| localization | 214269_at | FLJ22269 |
| localization | 209404_s_at | TMED7 |
| localization | 227873_at | C5orf14 |
| localization | 240036_at | SEC14L1 |
| localization | 224576_at | KIAA1181 |
| localization | 238034_at | CANX |
| localization | 202125_s_at | ALS2CR3 |
| localization | 212833_at | LOC91137 |
| localization | 227761_at | MYO5A |
| localization | 1558487_a_at | TMED4 |
| localization | 228391_at | CYP4V2 |
| localization | 203325_s_at | COL5A1 |
| localization | 226831_at | LOC91137 |
| establishment of localization | 211980_at | COL4A1 |
| establishment of localization | 201438_at | COL6A3 |
| establishment of localization | 214269_at | FLJ22269 |
| establishment of localization | 209404_s_at | TMED7 |
| establishment of localization | 227873_at | C5orf14 |
| establishment of localization | 240036_at | SEC14L1 |
| establishment of localization | 224576_at | KIAA1181 |
| establishment of localization | 238034_at | CANX |
| establishment of localization | 202125_s_at | ALS2CR3 |
| establishment of localization | 212833_at | LOC91137 |
| establishment of localization | 227761_at | MYO5A |
| establishment of localization | 1558487_a_at | TMED4 |
| establishment of localization | 228391_at | CYP4V2 |
| establishment of localization | 203325_s_at | COL5A1 |
| establishment of localization | 226831_at | LOC91137 |
| protein metabolism | 226538_at | MAN2A1 |
| protein metabolism | 205105_at | MAN2A1 |
| protein metabolism | 201307_at | septin 11 |
| protein metabolism | 1560065_at | PAIP2 |
| protein metabolism | 227413_at | UBLCP1 |
| protein metabolism | 235103_at | MAN2A1 |
| protein metabolism | 238034_at | CANX |
| protein metabolism | 40420_at | STK10 |
| protein metabolism | 201041_s_at | DUSP1 |
| protein metabolism | 203501_at | PGCP |
| protein metabolism | 229285_at | RNASEL |
| protein metabolism | 200906_s_at | KIAA0992 |
| transport | 211980_at | COL4A1 |
| transport | 201438_at | COL6A3 |
| transport | 214269_at | FLJ22269 |
| transport | 209404_s_at | TMED7 |
| transport | 227873_at | C5orf14 |
| transport | 240036_at | SEC14L1 |
| transport | 224576_at | KIAA1181 |
| transport | 202125_s_at | ALS2CR3 |
| transport | 212833_at | LOC91137 |
| transport | 227761_at | MYO5A |
| transport | 1558487_a_at | TMED4 |
| transport | 228391_at | CYP4V2 |
| transport | 203325_s_at | COL5A1 |
| transport | 226831_at | LOC91137 |
| cell communication | 225275_at | EDIL3 |
| cell communication | 201438_at | COL6A3 |
| cell communication | 218323_at | RHOT1 |
| cell communication | 230398_at | TNS4 |
| cell communication | 208005_at | NTN1 |
| cell communication | 210809_s_at | POSTN |
| cell communication | 212895_s_at | ABR |
| cell communication | 1558487_a_at | TMED4 |
| cell communication | 203325_s_at | COL5A1 |
| cell communication | 1556687_a_at | CLDN10 |
| cell fraction | 226538_at | MAN2A1 |
| cell fraction | 205105_at | MAN2A1 |
| cell fraction | 205407_at | RECK |
| cell fraction | 235103_at | MAN2A1 |
| cell fraction | 208005_at | NTN1 |
| macromolecule metabolism | 226538_at | MAN2A1 |
| macromolecule metabolism | 205105_at | MAN2A1 |
| macromolecule metabolism | 201307_at | septin 11 |
| macromolecule metabolism | 201944_at | HEXB |
| macromolecule metabolism | 1560065_at | PAIP2 |
| macromolecule metabolism | 227413_at | UBLCP1 |
| macromolecule metabolism | 235103_at | MAN2A1 |
| macromolecule metabolism | 238034_at | CANX |
| macromolecule metabolism | 40420_at | STK10 |
| macromolecule metabolism | 201041_s_at | DUSP1 |
| macromolecule metabolism | 203501_at | PGCP |
| macromolecule metabolism | 229285_at | RNASEL |
| macromolecule metabolism | 200906_s_at | KIAA0992 |
| membrane-bound organelle | 226538_at | MAN2A1 |
| membrane-bound organelle | 205105_at | MAN2A1 |
| membrane-bound organelle | 203823_at | RGS3 |
| membrane-bound organelle | 201413_at | HSD17B4 |
| membrane-bound organelle | 1554583_a_at | MGC50559 |
| membrane-bound organelle | 220347_at | C17orf31 |
| membrane-bound organelle | 205022_s_at | CHES1 |
| membrane-bound organelle | 212737_at | GM2A |
| membrane-bound organelle | 209404_s_at | TMED7 |
| membrane-bound organelle | 201944_at | HEXB |
| membrane-bound organelle | 240036_at | SEC14L1 |
| membrane-bound organelle | 233852_at | POLH |
| membrane-bound organelle | 201924_at | AFF1 |
| membrane-bound organelle | 224576_at | KIAA1181 |
| membrane-bound organelle | 230031_at | HSPA5 |
| membrane-bound organelle | 235103_at | MAN2A1 |
| membrane-bound organelle | 223512_at | SARA2 |
| membrane-bound organelle | 238276_at | CCNL1 |
| membrane-bound organelle | 204995_at | CDK5R1 |
| membrane-bound organelle | 208070_s_at | REV3L |
| membrane-bound organelle | 209348_s_at | MAF |
| membrane-bound organelle | 224928_at | SET7 |
| membrane-bound organelle | 200906_s_at | KIAA0992 |
| membrane-bound organelle | 1558487_a_at | TMED4 |
| membrane-bound organelle | 228391_at | CYP4V2 |
| membrane-bound organelle | 205927_s_at | CTSE |
| membrane-bound organelle | 1558184_s_at | ZNF17 |
| membrane-bound organelle | 211569_s_at | HADHSC |
| intracellular membrane-bound | 226538_at | MAN2A1 |
| organelle | | |
| intracellular membrane-bound | 205105_at | MAN2A1 |
| organelle | | |
| intracellular membrane-bound | 203823_at | RGS3 |
| organelle | | |
| intracellular membrane-bound | 201413_at | HSD17B4 |
| organelle | | |
| intracellular membrane-bound | 1554583_a_at | MGC50559 |
| organelle | | |
| intracellular membrane-bound | 220347_at | C17orf31 |
| organelle | | |
| intracellular membrane-bound | 205022_s_at | CHES1 |
| organelle | | |
| intracellular membrane-bound | 212737_at | GM2A |
| organelle | | |
| intracellular membrane-bound | 209404_s_at | TMED7 |
| organelle | | |
| intracellular membrane-bound | 201944_at | HEXB |
| organelle | | |
| intracellular membrane-bound | 240036_at | SEC14L1 |
| organelle | | |
| intracellular membrane-bound | 233852_at | POLH |
| organelle | | |
| intracellular membrane-bound | 201924_at | AFF1 |
| organelle | | |
| intracellular membrane-bound | 224576_at | KIAA1181 |
| organelle | | |
| intracellular membrane-bound | 230031_at | HSPA5 |
| organelle | | |
| intracellular membrane-bound | 235103_at | MAN2A1 |
| organelle | | |
| intracellular membrane-bound | 223512_at | SARA2 |
| organelle | | |
| intracellular membrane-bound | 238276_at | CCNL1 |
| organelle | | |
| intracellular membrane-bound | 204995_at | CDK5R1 |
| organelle | | |
| intracellular membrane-bound | 208070_s_at | REV3L |
| organelle | | |
| intracellular membrane-bound | 209348_s_at | MAF |
| organelle | | |
| intracellular membrane-bound | 224928_at | SET7 |
| organelle | | |
| intracellular membrane-bound | 200906_s_at | KIAA0992 |
| organelle | | |
| intracellular membrane-bound | 1558487_a_at | TMED4 |
| organelle | | |
| intracellular membrane-bound | 228391_at | CYP4V2 |
| organelle | | |
| intracellular membrane-bound | 205927_s_at | CTSE |
| organelle | | |
| intracellular membrane-bound | 1558184_s_at | ZNF17 |
| organelle | | |
| intracellular membrane-bound | 211569_s_at | HADHSC |
| organelle | | |
| cellular macromolecule metabolism | 226538_at | MAN2A1 |
| cellular macromolecule metabolism | 205105_at | MAN2A1 |
| cellular macromolecule metabolism | 1560065_at | PAIP2 |
| cellular macromolecule metabolism | 227413_at | UBLCP1 |
| cellular macromolecule metabolism | 235103_at | MAN2A1 |
| cellular macromolecule metabolism | 238034_at | CANX |
| cellular macromolecule metabolism | 40420_at | STK10 |
| cellular macromolecule metabolism | 201041_s_at | DUSP1 |
| cellular macromolecule metabolism | 203501_at | PGCP |
| cellular macromolecule metabolism | 229285_at | RNASEL |
| cellular macromolecule metabolism | 200906_s_at | KIAA0992 |
| cellular protein metabolism | 226538_at | MAN2A1 |
| cellular protein metabolism | 205105_at | MAN2A1 |
| cellular protein metabolism | 1560065_at | PAIP2 |
| cellular protein metabolism | 227413_at | UBLCP1 |
| cellular protein metabolism | 235103_at | MAN2A1 |
| cellular protein metabolism | 238034_at | CANX |
| cellular protein metabolism | 40420_at | STK10 |
| cellular protein metabolism | 201041_s_at | DUSP1 |
| cellular protein metabolism | 203501_at | PGCP |
| cellular protein metabolism | 229285_at | RNASEL |
| cellular protein metabolism | 200906_s_at | KIAA0992 |
| primary metabolism | 226538_at | MAN2A1 |
| primary metabolism | 205105_at | MAN2A1 |
| primary metabolism | 201307_at | septin 11 |
| primary metabolism | 201413_at | HSD17B4 |
| primary metabolism | 201944_at | HEXB |
| primary metabolism | 1560065_at | PAIP2 |
| primary metabolism | 227413_at | UBLCP1 |
| primary metabolism | 235103_at | MAN2A1 |
| primary metabolism | 238034_at | CANX |
| primary metabolism | 40420_at | STK10 |
| primary metabolism | 238276_at | CCNL1 |
| primary metabolism | 201041_s_at | DUSP1 |
| primary metabolism | 203501_at | PGCP |
| primary metabolism | 229285_at | RNASEL |
| primary metabolism | 209348_s_at | MAF |
| primary metabolism | 224928_at | SET7 |
| primary metabolism | 200906_s_at | KIAA0992 |
| primary metabolism | 1558184_s_at | ZNF17 |
| primary metabolism | 211569_s_at | HADHSC |
| organelle | 226538_at | MAN2A1 |
| organelle | 205105_at | MAN2A1 |
| organelle | 201307_at | septin 11 |
| organelle | 203823_at | RGS3 |
| organelle | 201413_at | HSD17B4 |
| organelle | 1554583_a_at | MGC50559 |
| organelle | 220347_at | C17orf31 |
| organelle | 205022_s_at | CHES1 |
| organelle | 212737_at | GM2A |
| organelle | 201215_at | PLS3 |
| organelle | 209404_s_at | TMED7 |
| organelle | 201944_at | HEXB |
| organelle | 240036_at | SEC14L1 |
| organelle | 233852_at | POLH |
| organelle | 201924_at | AFF1 |
| organelle | 224576_at | KIAA1181 |
| organelle | 230031_at | HSPA5 |
| organelle | 235103_at | MAN2A1 |
| organelle | 223512_at | SARA2 |
| organelle | 238276_at | CCNL1 |
| organelle | 204995_at | CDK5R1 |
| organelle | 208070_s_at | REV3L |
| organelle | 201061_s_at | STOM |
| organelle | 209348_s_at | MAF |
| organelle | 224928_at | SET7 |
| organelle | 200906_s_at | KIAA0992 |
| organelle | 1558487_a_at | TMED4 |
| organelle | 228391_at | CYP4V2 |
| organelle | 205927_s_at | CTSE |
| organelle | 1558184_s_at | ZNF17 |
| organelle | 211569_s_at | HADHSC |
| intracellular organelle | 226538_at | MAN2A1 |
| intracellular organelle | 205105_at | MAN2A1 |
| intracellular organelle | 201307_at | septin 11 |
| intracellular organelle | 203823_at | RGS3 |
| intracellular organelle | 201413_at | HSD17B4 |
| intracellular organelle | 1554583_a_at | MGC50559 |
| intracellular organelle | 220347_at | C17orf31 |
| intracellular organelle | 205022_s_at | CHES1 |
| intracellular organelle | 212737_at | GM2A |
| intracellular organelle | 201215_at | PLS3 |
| intracellular organelle | 209404_s_at | TMED7 |
| intracellular organelle | 201944_at | HEXB |
| intracellular organelle | 240036_at | SEC14L1 |
| intracellular organelle | 233852_at | POLH |
| intracellular organelle | 201924_at | AFF1 |
| intracellular organelle | 224576_at | KIAA1181 |
| intracellular organelle | 230031_at | HSPA5 |
| intracellular organelle | 235103_at | MAN2A1 |
| intracellular organelle | 223512_at | SARA2 |
| intracellular organelle | 238276_at | CCNL1 |
| intracellular organelle | 204995_at | CDK5R1 |
| intracellular organelle | 208070_s_at | REV3L |
| intracellular organelle | 201061_s_at | STOM |
| intracellular organelle | 209348_s_at | MAF |
| intracellular organelle | 224928_at | SET7 |
| intracellular organelle | 200906_s_at | KIAA0992 |
| intracellular organelle | 1558487_a_at | TMED4 |
| intracellular organelle | 228391_at | CYP4V2 |
| intracellular organelle | 205927_s_at | CTSE |
| intracellular organelle | 1558184_s_at | ZNF17 |
| intracellular organelle | 211569_s_at | HADHSC |
| metabolism | 226538_at | MAN2A1 |
| metabolism | 205105_at | MAN2A1 |
| metabolism | 201307_at | septin 11 |
| metabolism | 201413_at | HSD17B4 |
| metabolism | 227873_at | C5orf14 |
| metabolism | 201944_at | HEXB |
| metabolism | 1560065_at | PAIP2 |
| metabolism | 227413_at | UBLCP1 |
| metabolism | 235103_at | MAN2A1 |
| metabolism | 238034_at | CANX |
| metabolism | 40420_at | STK10 |
| metabolism | 238276_at | CCNL1 |
| metabolism | 201041_s_at | DUSP1 |
| metabolism | 227761_at | MYO5A |
| metabolism | 203501_at | PGCP |
| metabolism | 229285_at | RNASEL |
| metabolism | 209348_s_at | MAF |
| metabolism | 224928_at | SET7 |
| metabolism | 200906_s_at | KIAA0992 |
| metabolism | 228391_at | CYP4V2 |
| metabolism | 1558184_s_at | ZNF17 |
| metabolism | 211569_s_at | HADHSC |
| metabolism | 219973_at | ARSJ |
| extracellular region | 202310_s_at | COL1A1 |
| extracellular region | 201438_at | COL6A3 |
| extracellular region | 201506_at | TGFBI |
| extracellular region | 230170_at | OSM |
| extracellular region | 208005_at | NTN1 |
| extracellular region | 210809_s_at | POSTN |
| extracellular region | 203501_at | PGCP |
| non-membrane-bound organelle | 201307_at | septin 11 |
| non-membrane-bound organelle | 220347_at | C17orf31 |
| non-membrane-bound organelle | 201215_at | PLS3 |
| non-membrane-bound organelle | 201061_s_at | STOM |
| non-membrane-bound organelle | 209348_s_at | MAF |
| non-membrane-bound organelle | 200906_s_at | KIAA0992 |
| intracellular non-membrane-bound | 201307_at | septin 11 |
| organelle | | |
| intracellular non-membrane-bound | 220347_at | C17orf31 |
| organelle | | |
| intracellular non-membrane-bound | 201215_at | PLS3 |
| organelle | | |
| intracellular non-membrane-bound | 201061_s_at | STOM |
| organelle | | |
| intracellular non-membrane-bound | 209348_s_at | MAF |
| organelle | | |
| intracellular non-membrane-bound | 200906_s_at | KIAA0992 |
| organelle | | |
| cellular metabolism | 226538_at | MAN2A1 |
| cellular metabolism | 205105_at | MAN2A1 |
| cellular metabolism | 201413_at | HSD17B4 |
| cellular metabolism | 227873_at | C5orf14 |
| cellular metabolism | 201944_at | HEXB |
| cellular metabolism | 1560065_at | PAIP2 |
| cellular metabolism | 227413_at | UBLCP1 |
| cellular metabolism | 235103_at | MAN2A1 |
| cellular metabolism | 238034_at | CANX |
| cellular metabolism | 40420_at | STK10 |
| cellular metabolism | 238276_at | CCNL1 |
| cellular metabolism | 201041_s_at | DUSP1 |
| cellular metabolism | 227761_at | MYO5A |
| cellular metabolism | 203501_at | PGCP |
| cellular metabolism | 229285_at | RNASEL |
| cellular metabolism | 209348_s_at | MAF |
| cellular metabolism | 224928_at | SET7 |
| cellular metabolism | 200906_s_at | KIAA0992 |
| cellular metabolism | 228391_at | CYP4V2 |
| cellular metabolism | 1558184_s_at | ZNF17 |
| cellular metabolism | 211569_s_at | HADHSC |
| structural molecule activity | 211980_at | COL4A1 |
| structural molecule activity | 202310_s_at | COL1A1 |
| structural molecule activity | 208005_at | NTN1 |
| structural molecule activity | 203325_s_at | COL5A1 |
| structural molecule activity | 1556687_a_at | CLDN10 |
| extracellular matrix (sensu Metazoa) | 202310_s_at | COL1A1 |
| extracellular matrix (sensu Metazoa) | 201438_at | COL6A3 |
| extracellular matrix (sensu Metazoa) | 201506_at | TGFBI |
| extracellular matrix (sensu Metazoa) | 208005_at | NTN1 |
| extracellular matrix (sensu Metazoa) | 210809_s_at | POSTN |
| extracellular matrix | 202310_s_at | COL1A1 |
| extracellular matrix | 201438_at | COL6A3 |
| extracellular matrix | 201506_at | TGFBI |
| extracellular matrix | 208005_at | NTN1 |
| extracellular matrix | 210809_s_at | POSTN |
| nucleus | 203823_at | RGS3 |
| nucleus | 1554583_a_at | MGC50559 |
| nucleus | 220347_at | C17orf31 |
| nucleus | 205022_s_at | CHES1 |
| nucleus | 233852_at | POLH |
| nucleus | 201924_at | AFF1 |
| nucleus | 238276_at | CCNL1 |
| nucleus | 204995_at | CDK5R1 |
| nucleus | 208070_s_at | REV3L |
| nucleus | 209348_s_at | MAF |
| nucleus | 224928_at | SET7 |
| nucleus | 200906_s_at | KIAA0992 |
| nucleus | 1558184_s_at | ZNF17 |
| regulation of biological process | 1560065_at | PAIP2 |
| regulation of biological process | 205407_at | RECK |
| regulation of biological process | 238276_at | CCNL1 |
| regulation of biological process | 203619_s_at | FAIM2 |
| regulation of biological process | 209348_s_at | MAF |
| regulation of biological process | 1558487_a_at | TMED4 |
| regulation of biological process | 1558184_s_at | ZNF17 |
| regulation of cellular process | 1560065_at | PAIP2 |
| regulation of cellular process | 205407_at | RECK |
| regulation of cellular process | 238276_at | CCNL1 |
| regulation of cellular process | 203619_s_at | FAIM2 |
| regulation of cellular process | 209348_s_at | MAF |
| regulation of cellular process | 1558487_a_at | TMED4 |
| regulation of cellular process | 1558184_s_at | ZNF17 |
| transporter activity | 214269_at | FLJ22269 |
| transporter activity | 209404_s_at | TMED7 |
| transporter activity | 227873_at | C5orf14 |
| transporter activity | 240036_at | SEC14L1 |
| transporter activity | 221584_s_at | KCNMA1 |
| transporter activity | 202125_s_at | ALS2CR3 |
| transporter activity | 1558487_a_at | TMED4 |
| regulation of physiological process | 1560065_at | PAIP2 |
| regulation of physiological process | 205407_at | RECK |
| regulation of physiological process | 238276_at | CCNL1 |
| regulation of physiological process | 203619_s_at | FAIM2 |
| regulation of physiological process | 209348_s_at | MAF |
| regulation of physiological process | 1558184_s_at | ZNF17 |
| regulation of cellular physiological | 1560065_at | PAIP2 |
| process | | |
| regulation of cellular physiological | 205407_at | RECK |
| process | | |
| regulation of cellular physiological | 238276_at | CCNL1 |
| process | | |
| regulation of cellular physiological | 203619_s_at | FAIM2 |
| process | | |
| regulation of cellular physiological | 209348_s_at | MAF |
| process | | |
| regulation of cellular physiological | 1558184_s_at | ZNF17 |
| process | | |
| development | 225275_at | EDIL3 |
| development | 201438_at | COL6A3 |
| development | 202766_s_at | FBN1 |
| development | 208005_at | NTN1 |
| development | 210809_s_at | POSTN |
| cell adhesion | 225275_at | EDIL3 |
| cell adhesion | 201438_at | COL6A3 |
| cell adhesion | 210809_s_at | POSTN |
| cell adhesion | 203325_s_at | COL5A1 |
| cell adhesion | 1556687_a_at | CLDN10 |
| nucleic acid binding | 220347_at | C17orf31 |
| nucleic acid binding | 201924_at | AFF1 |
| nucleic acid binding | 210966_x_at | LARP1 |
| nucleic acid binding | 212193_s_at | LARP1 |
| nucleic acid binding | 209348_s_at | MAF |
| protein modification | 227413_at | UBLCP1 |
| protein modification | 40420_at | STK10 |
| protein modification | 201041_s_at | DUSP1 |
| protein modification | 229285_at | RNASEL |
| protein modification | 200906_s_at | KIAA0992 |
| biopolymer modification | 227413_at | UBLCP1 |
| biopolymer modification | 40420_at | STK10 |
| biopolymer modification | 201041_s_at | DUSP1 |
| biopolymer modification | 229285_at | RNASEL |
| biopolymer modification | 200906_s_at | KIAA0992 |
| biopolymer metabolism | 227413_at | UBLCP1 |
| biopolymer metabolism | 40420_at | STK10 |
| biopolymer metabolism | 201041_s_at | DUSP1 |
| biopolymer metabolism | 203501_at | PGCP |
| biopolymer metabolism | 229285_at | RNASEL |
| biopolymer metabolism | 224928_at | SET7 |
| biopolymer metabolism | 200906_s_at | KIAA0992 |
| endoplasmic reticulum | 209404_s_at | TMED7 |
| endoplasmic reticulum | 224576_at | KIAA1181 |
| endoplasmic reticulum | 230031_at | HSPA5 |
| endoplasmic reticulum | 223512_at | SARA2 |
| endoplasmic reticulum | 1558487_a_at | TMED4 |
| endoplasmic reticulum | 228391_at | CYP4V2 |
| enzyme regulator activity | 203823_at | RGS3 |
| enzyme regulator activity | 212737_at | GM2A |
| enzyme regulator activity | 212895_s_at | ABR |
| enzyme regulator activity | 227947_at | PHACTR2 |
| enzyme regulator activity | 242277_at | PHACTR2 |
| nucleobase\, nucleoside\, | 238276_at | CCNL1 |
| nucleotide and nucleic acid | | |
| metabolism | | |
| nucleobase\, nucleoside\, | 229285_at | RNASEL |
| nucleotide and nucleic acid | | |
| metabolism | | |
| nucleobase\, nucleoside\, | 209348_s_at | MAF |
| nucleotide and nucleic acid | | |
| metabolism | | |
| nucleobase\, nucleoside\, | 224928_at | SET7 |
| nucleotide and nucleic acid | | |
| metabolism | | |
| nucleobase\, nucleoside\, | 1558184_s_at | ZNF17 |
| nucleotide and nucleic acid | | |
| metabolism | | |

The performance of this refractory gene list on the original training set is shown in Table 3. The overall accuracy of the refractory gene list during LOOCV ranged from 84-88% with 75-88% of the refractory samples correctly identified and 92-100% of the sensitive samples correctly identified by the predictive algorithm. Accuracy is the proportion of true results (both true positives and true negatives) in the population. It is a parameter of the test.

TABLE 3
Performance of refractory gene list on training set.

| | | | | Misclassification |
|---|---|---|---|---|
| Predictor | OVERALL(25) | SENS(13) | RES(12) | Rate |
| CCP | 88% | 100% | 75% | p < 0.001 |
| DLDA | 88% | 100% | 75% | p < 0.001 |
| 1-NN | 88% | 92% | 83% | p = 0.001 |
| 3-NN | 84% | 92% | 75% | p = 0.001 |
| NC | 88% | 100% | 75% | p < 0.001 |
| SVM | 84% | 92% | 75% | p = 0.002 |

The refractory gene list was applied to an independent test set to further validate the predictive nature of the refractory gene list. The test set comprised of 7 subject samples whose tumors were refractory to chemotherapy and 6 subject samples whose tumors were sensitive to chemotherapy. The overall accuracy ranged from 77 to 92% with 83-100% of the sensitive samples and 71-86% of the refractory samples correctly predicted (Table 4).

TABLE 4
Prediction accuracy of refractory gene list on test samples.

| Predictor | OVERALL(n = 10) | SENS(n = 6) | REF(n = 7) |
|---|---|---|---|
| CCP | 92% | 100% | 86% |
| DLDA | 92% | 100% | 86% |
| 1-NN | 85% | 83% | 86% |
| 3-NN | 77% | 83% | 71% |
| NC | 92% | 100% | 86% |
| SVM | 92% | 83% | 86% |

The data indicate that the 105 chemorefractory specific molecules can be used to predict chemorefraction in subjects with ovarian cancer with high specificity and sensitivity.

### EXAMPLE 3

### Development of Predictive Chemoresistant Gene Signature

This example provides methods used to identify 31 chemoresistant specific molecules that can be used to predict chemoresponsiveness, such as chemoresistance, in subjects with ovarian cancer.

The training set to develop the predictive chemoresistant gene signature (resistant gene list) comprised of 10 subject samples whose tumors were resistant to chemotherapy and 13 subject samples whose tumors were sensitive to chemotherapy. The list was refined to include only genes used in all LOOCV iterations. This refinement yielded a 31-gene signature list as shown in Table 5. The function and/or location of the respective genes are provided in Table 6.

TABLE 5
Chemoresistant gene signature profile.

| | | Fold Change | UniGene | | | |
|---|---|---|---|---|---|---|
| AFFYMETRIX ® | Parametric | in | ID | GENE | LocusLINK | |
| PROBE ID | p-value | RESISTANT | Number | SYMBOL | ID | GENE Name |
| 1566512_at | 0.000246 | −1.523091423 | Hs.159711 | GNG4 | 2786 | Hypothetical protein |
| | | | | | | LOC200169 |
| 201147_s_at | 0.000315 | 2.548387097 | Hs.297324 | TIMP3 | 7078 | TIMP metallopeptidase |
| | | | | | | inhibitor 3 (Sorsby fundus |
| | | | | | | dystrophy, |
| | | | | | | pseudoinflammatory) |
| 201310_s_at | 0.000308 | 4.008130081 | Hs.483067 | C5orf13 | 9315 | chromosome 5 open reading |
| | | | | | | frame 13 |
| 201340_s_at | 4.30E−05 | 2.967159278 | Hs.104925 | ENC1 | 8507 | ectodermal-neural cortex |
| | | | | | | (with BTB-like domain) |
| 201341_at | 0.000169 | 2.049141031 | Hs.104925 | ENC1 | 8507 | ectodermal-neural cortex |
| | | | | | | (with BTB-like domain) |
| 201669_s_at | 0.000346 | 3.478952292 | Hs.519909 | MARCKS | 4082 | myristoylated alanine-rich |
| | | | | | | protein kinase C substrate |
| 201915_at | 0.000196 | 2.361522199 | Hs.529957 | SEC63 | 11231 | SEC63-like (S. cerevisiae) |
| 202052_s_at | 5.10E−05 | 2.901684115 | Hs.431400 | RAI14 | 26064 | retinoic acid induced 14 |
| 202733_at | 7.70E−05 | 2.771155596 | Hs.519568 | P4HA2 | 8974 | procollagen-proline, 2- |
| | | | | | | oxoglutarate 4-dioxygenase |
| | | | | | | (proline 4-hydroxylase), |
| | | | | | | alpha polypeptide II |
| 203370_s_at | 0.000112 | 1.422018349 | Hs.533040 | PDLM7 | 9260 | PDZ and LEVI domain 7 |
| | | | | | | (enigma) |
| 203570_at | 0.000188 | 3.120817844 | Hs.65436 | LOXL1 | 4016 | lysyl oxidase-like 1 |
| 204117_at | 0.000152 | 1.961195929 | Hs.436564 | PREP | 5550 | prolyl endopeptidase |
| 204270_at | 0.000109 | −2.019366197 | Hs.467529 | SKI | 6497 | v-ski sarcoma viral oncogene |
| | | | | | | homolog (avian) |
| 212385_at | 0.000277 | 1.860176991 | Hs.200285 | TCF4 | 6925 | Transcription factor 4 |
| 212899_at | 0.000286 | 2.495362563 | Hs.193251 | CDC2L6 | 23097 | cell division cycle 2-like 6 |
| | | | | | | (CDK8-like) |
| 213062_at | 0.00029 | 1.88091716 | Hs.351573 | NTAN1 | 123803 | N-terminal asparagine |
| | | | | | | amidase |
| 213906_at | 0.000242 | 2.266506603 | Hs.445898 | MYBL1 | 4603 | v-myb myeloblastosis viral |
| | | | | | | oncogene homolog (avian)- |
| | | | | | | like 1 |
| 218196_at | 0.000293 | 2.384955752 | Hs.226780 | OSTM1 | 28962 | osteopetrosis associated |
| | | | | | | transmembrane protein 1 |
| 219479_at | 0.000403 | 2.208633094 | Hs.408629 | KDELC1 | 79070 | KDEL (Lys-Asp-Glu-Leu) |
| | | | | | | containing 1 |
| 221021_s_at | 0.000172 | 2.121268657 | Hs.472667 | CTNNBL1 | 56259 | catenin, beta like 1 /// |
| | | | | | | catenin, beta like 1 |
| 221503_s_at | 0.000169 | 1.487778959 | Hs.527919 | KPNA3 | 3839 | karyopherin alpha 3 |
| | | | | | | (importin alpha 4) |
| 222670_s_at | 0.000362 | 2.022573363 | Hs.169487 | MAFB | 9935 | v-maf musculoaponeurotic |
| | | | | | | fibrosarcoma oncogene |
| | | | | | | homolog B (avian) |
| 224733_at | 0.000346 | 2.077308518 | Hs.298198 | CKLFSF3 | 123920 | chemokine-like factor |
| | | | | | | superfamily 3 |
| 225664_at | 0.000321 | 4.706035606 | Hs.101302 | COL12A1 | 1303 | collagen, type XII, alpha 1 |
| 227376_at | 0.000335 | 3.255005269 | Hs.21509 | | 402485 | Hypothetical LOC401328 |
| 228033_at | 0.00024 | 4.230958231 | Hs.416375 | E2F7 | 144455 | E2F transcription factor 7 |
| 229644_at | 0.000147 | 1.96031746 | Hs.436564 | PREP | 5550 | Prolyl endopeptidase |
| 238617_at | 0.000332 | −1.419282511 | Hs.143134 | | | CDNA FLJ38181 fis, clone |
| | | | | | | FCBBF1000125 |
| 242418_at | 2.50E−05 | 3.97804878 | | | | |
| 37950_at | 0.000316 | 1.550239234 | Hs.436564 | PREP | 5550 | prolyl endopeptidase |
| | 5.90E−05 | 2.331853496 | | | | |

Genes with a positive fold change are up-regulated in chemoresistant ovarian tumors and genes with a negative fold change are down-regulated.

TABLE 6
Function and/or location of chemoresistant specific molecules.

| | AFFYMETRIX ® | |
|---|---|---|
| FUNCTION/LOCATION | PROBE ID | Gene NAME |
| membrane-bound organelle | 202733_at | P4HA2 |
| membrane-bound organelle | 221021_s_at | CTNNBL1 |
| membrane-bound organelle | 222670_s_at | MAFB |
| membrane-bound organelle | 201340_s_at | ENC1 |
| membrane-bound organelle | 204270_at | SKI |
| membrane-bound organelle | 212385_at | TCF4 |
| membrane-bound organelle | 201341_at | ENC1 |
| membrane-bound organelle | 213906_at | MYBL1 |
| membrane-bound organelle | 221503_s_at | KPNA3 |
| intracellular membrane-bound | 202733_at | P4HA2 |
| organelle | | |
| intracellular membrane-bound | 221021_s_at | CTNNBL1 |
| organelle | | |
| intracellular membrane-bound | 222670_s_at | MAFB |
| organelle | | |
| intracellular membrane-bound | 201340_s_at | ENC1 |
| organelle | | |
| intracellular membrane-bound | 204270_at | SKI |
| organelle | | |
| intracellular membrane-bound | 212385_at | TCF4 |
| organelle | | |
| intracellular membrane-bound | 201341_at | ENC1 |
| organelle | | |
| intracellular membrane-bound | 213906_at | MYBL1 |
| organelle | | |
| intracellular membrane-bound | 221503_s_at | KPNA3 |
| organelle | | |
| nucleus | 221021_s_at | CTNNBL1 |
| nucleus | 222670_s_at | MAFB |
| nucleus | 201340_s_at | ENC1 |
| nucleus | 204270_at | SKI |
| nucleus | 212385_at | TCF4 |
| nucleus | 201341_at | ENC1 |
| nucleus | 213906_at | MYBL1 |
| nucleus | 221503_s_at | KPNA3 |
| molecular_function | 202733_at | P4HA2 |
| molecular_function | 221021_s_at | CTNNBL1 |
| molecular_function | 1566512_at | GNG4 |
| molecular_function | 213062_at | NTAN1 |
| molecular_function | 222670_s_at | MAFB |
| molecular_function | 203370_s_at | PDLIM7 |
| molecular_function | 224733_at | CKLFSF3 |
| molecular_function | 201340_s_at | ENC1 |
| molecular_function | 225664_at | COL12A1 |
| molecular_function | 204270_at | SKI |
| molecular_function | 201147_s_at | TIMP3 |
| molecular_function | 201915_at | SEC63 |
| molecular_function | 201341_at | ENC1 |
| molecular_function | 213906_at | MYBL1 |
| protein binding | 202733_at | P4HA2 |
| protein binding | 203370_s_at | PDLM7 |
| protein binding | 224733_at | CKLFSF3 |
| protein binding | 201340_s_at | ENC1 |
| protein binding | 225664_at | COL12A1 |
| protein binding | 204270_at | SKI |
| protein binding | 201915_at | SEC63 |
| protein binding | 201341_at | ENC1 |
| binding | 202733_at | P4HA2 |
| binding | 221021_s_at | CTNNBL1 |
| binding | 222670_s_at | MAFB |
| binding | 203370_s_at | PDLM7 |
| binding | 224733_at | CKLFSF3 |
| binding | 201340_s_at | ENC1 |
| binding | 225664_at | COL12A1 |
| binding | 204270_at | SKI |
| binding | 201915_at | SEC63 |
| binding | 201341_at | ENC1 |
| binding | 213906_at | MYBL1 |
| cellular_component | 202733_at | P4HA2 |
| cellular_component | 221021_s_at | CTNNBL1 |
| cellular_component | 1566512_at | GNG4 |
| cellular_component | 222670_s_at | MAFB |
| cellular_component | 37950_at | PREP |
| cellular_component | 224733_at | CKLFSF3 |
| cellular_component | 201340_s_at | ENC1 |
| cellular_component | 225664_at | COL12A1 |
| cellular_component | 204270_at | SKI |
| cellular_component | 201147_s_at | TIMP3 |
| cellular_component | 212385_at | TCF4 |
| cellular_component | 204117_at | PREP |
| cellular_component | 218196_at | OSTM1 |
| cellular_component | 201341_at | ENC1 |
| cellular_component | 203570_at | LOXL1 |
| cellular_component | 213906_at | MYBL1 |
| cellular_component | 201669_s_at | MARCKS |
| cellular_component | 229644_at | PREP |
| cellular_component | 221503_s_at | KPNA3 |
| intracellular | 202733_at | P4HA2 |
| intracellular | 221021_s_at | CTNNBL1 |
| intracellular | 222670_s_at | MAFB |
| intracellular | 37950_at | PREP |
| intracellular | 201340_s_at | ENC1 |
| intracellular | 225664_at | COL12A1 |
| intracellular | 204270_at | SKI |
| intracellular | 212385_at | TCF4 |
| intracellular | 204117_at | PREP |
| intracellular | 201341_at | ENC1 |
| intracellular | 213906_at | MYBL1 |
| intracellular | 201669_s_at | MARCKS |
| intracellular | 229644_at | PREP |
| intracellular | 221503_s_at | KPNA3 |
| cell | 202733_at | P4HA2 |
| cell | 221021_s_at | CTNNBL1 |
| cell | 1566512_at | GNG4 |
| cell | 222670_s_at | MAFB |
| cell | 37950_at | PREP |
| cell | 224733_at | CKLFSF3 |
| cell | 201340_s_at | ENC1 |
| cell | 225664_at | COL12A1 |
| cell | 204270_at | SKI |
| cell | 212385_at | TCF4 |
| cell | 204117_at | PREP |
| cell | 218196_at | OSTM1 |
| cell | 201341_at | ENC1 |
| cell | 213906_at | MYBL1 |
| cell | 201669_s_at | MARCKS |
| cell | 229644_at | PREP |
| cell | 221503_s_at | KPNA3 |
| organelle | 202733_at | P4HA2 |
| organelle | 221021_s_at | CTNNBL1 |
| organelle | 222670_s_at | MAFB |
| organelle | 201340_s_at | ENC1 |
| organelle | 204270_at | SKI |
| organelle | 212385_at | TCF4 |
| organelle | 201341_at | ENC1 |
| organelle | 213906_at | MYBL1 |
| organelle | 201669_s_at | MARCKS |
| organelle | 221503_s_at | KPNA3 |
| intracellular organelle | 202733_at | P4HA2 |
| intracellular organelle | 221021_s_at | CTNNBL1 |
| intracellular organelle | 222670_s_at | MAFB |
| intracellular organelle | 201340_s_at | ENC1 |
| intracellular organelle | 204270_at | SKI |
| intracellular organelle | 212385_at | TCF4 |
| intracellular organelle | 201341_at | ENC1 |
| intracellular organelle | 213906_at | MYBL1 |
| intracellular organelle | 201669_s_at | MARCKS |
| intracellular organelle | 221503_s_at | KPNA3 |
| biological_process | 202733_at | P4HA2 |
| biological_process | 221021_s_at | CTNNBL1 |
| biological_process | 222670_s_at | MAFB |
| biological_process | 212899_at | CDC2L6 |
| biological_process | 37950_at | PREP |
| biological_process | 224733_at | CKLFSF3 |
| biological_process | 201340_s_at | ENC1 |
| biological_process | 225664_at | COL12A1 |
| biological_process | 204270_at | SKI |
| biological_process | 204117_at | PREP |
| biological_process | 201341_at | ENC1 |
| biological_process | 203570_at | LOXL1 |
| biological_process | 201669_s_at | MARCKS |
| biological_process | 229644_at | PREP |
| metabolism | 202733_at | P4HA2 |
| metabolism | 222670_s_at | MAFB |
| metabolism | 212899_at | CDC2L6 |
| metabolism | 37950_at | PREP |
| metabolism | 204117_at | PREP |
| metabolism | 203570_at | LOXL1 |
| metabolism | 229644_at | PREP |
| primary metabolism | 202733_at | P4HA2 |
| primary metabolism | 222670_s_at | MAFB |
| primary metabolism | 212899_at | CDC2L6 |
| primary metabolism | 37950_at | PREP |
| primary metabolism | 204117_at | PREP |
| primary metabolism | 203570_at | LOXL1 |
| primary metabolism | 229644_at | PREP |
| development | 222670_s_at | MAFB |
| development | 201340_s_at | ENC1 |
| development | 225664_at | COL12A1 |
| development | 204270_at | SKI |
| development | 201341_at | ENC1 |
| cytoplasm | 202733_at | P4HA2 |
| cytoplasm | 37950_at | PREP |
| cytoplasm | 225664_at | COL12A1 |
| cytoplasm | 204117_at | PREP |
| cytoplasm | 229644_at | PREP |
| protein metabolism | 202733_at | P4HA2 |
| protein metabolism | 212899_at | CDC2L6 |
| protein metabolism | 37950_at | PREP |
| protein metabolism | 204117_at | PREP |
| protein metabolism | 203570_at | LOXL1 |
| protein metabolism | 229644_at | PREP |
| macromolecule metabolism | 202733_at | P4HA2 |
| macromolecule metabolism | 212899_at | CDC2L6 |
| macromolecule metabolism | 37950_at | PREP |
| macromolecule metabolism | 204117_at | PREP |
| macromolecule metabolism | 203570_at | LOXL1 |
| macromolecule metabolism | 229644_at | PREP |
| physiological process | 202733_at | P4HA2 |
| physiological process | 221021_s_at | CTNNBL1 |
| physiological process | 222670_s_at | MAFB |
| physiological process | 212899_at | CDC2L6 |
| physiological process | 37950_at | PREP |
| physiological process | 224733_at | CKLFSF3 |
| physiological process | 225664_at | COL12A1 |
| physiological process | 204117_at | PREP |
| physiological process | 203570_at | LOXL1 |
| physiological process | 201669_s_at | MARCKS |
| physiological process | 229644_at | PREP |
| cellular process | 221021_s_at | CTNNBL1 |
| cellular process | 222670_s_at | MAFB |
| cellular process | 212899_at | CDC2L6 |
| cellular process | 37950_at | PREP |
| cellular process | 225664_at | COL12A1 |
| cellular process | 204270_at | SKI |
| cellular process | 204117_at | PREP |
| cellular process | 203570_at | LOXL1 |
| cellular process | 201669_s_at | MARCKS |
| cellular process | 229644_at | PREP |
| cellular metabolism | 222670_s_at | MAFB |
| cellular metabolism | 212899_at | CDC2L6 |
| cellular metabolism | 37950_at | PREP |
| cellular metabolism | 204117_at | PREP |
| cellular metabolism | 203570_at | LOXL1 |
| cellular metabolism | 229644_at | PREP |
| cellular physiological process | 221021_s_at | CTNNBL1 |
| cellular physiological process | 222670_s_at | MAFB |
| cellular physiological process | 212899_at | CDC2L6 |
| cellular physiological process | 37950_at | PREP |
| cellular physiological process | 225664_at | COL12A1 |
| cellular physiological process | 204117_at | PREP |
| cellular physiological process | 203570_at | LOXL1 |
| cellular physiological process | 201669_s_at | MARCKS |
| cellular physiological process | 229644_at | PREP |
| biopolymer metabolism | 212899_at | CDC2L6 |
| biopolymer metabolism | 37950_at | PREP |
| biopolymer metabolism | 204117_at | PREP |
| biopolymer metabolism | 203570_at | LOXL1 |
| biopolymer metabolism | 229644_at | PREP |
| cellular macromolecule metabolism | 212899_at | CDC2L6 |
| cellular macromolecule metabolism | 37950_at | PREP |
| cellular macromolecule metabolism | 204117_at | PREP |
| cellular macromolecule metabolism | 203570_at | LOXL1 |
| cellular macromolecule metabolism | 229644_at | PREP |
| cellular protein metabolism | 212899_at | CDC2L6 |
| cellular protein metabolism | 37950_at | PREP |
| cellular protein metabolism | 204117_at | PREP |
| cellular protein metabolism | 203570_at | LOXL1 |
| cellular protein metabolism | 229644_at | PREP |
| membrane | 1566512_at | GNG4 |
| membrane | 224733_at | CKLFSF3 |
| membrane | 218196_at | OSTM1 |
| membrane | 201669_s_at | MARCKS |
| membrane | 221503_s_at | KPNA3 |

The performance of this resistant gene signature list on the original training set is shown in Table 7. The overall accuracy of the resistant gene signature list during LOOCV was at 96% for all predictor algorithms used with 90% of the resistant samples correctly identified and 100% of the sensitive samples correctly identified.

TABLE 7
Performance of resistant gene list on training set.

| | | | | Misclassification |
|---|---|---|---|---|
| Predictor | OVERALL(23) | SENS(13) | RES(10) | Rate |
| CCP | 96% | 100% | 90% | p < 5e−04 |
| DLDA | 96% | 100% | 90% | p < 5e−04 |
| 1-NN | 96% | 100% | 90% | p < 5e−04 |
| 3-NN | 96% | 100% | 90% | p < 5e−04 |
| NC | 96% | 100% | 90% | p < 5e−04 |
| SVM | 96% | 100% | 90% | p < 5e−04 |

This resistance-associated gene list was then applied to an independent test set to further validate the predictive nature of the gene list. The test set comprised of 4 subject samples whose tumors were resistant to chemotherapy and 6 subject samples whose tumors were sensitive to chemotherapy. The overall accuracy ranged from 80 to 90% with 83-100% of the sensitive samples and 75% of the resistant samples correctly predicted (Table 8).

TABLE 8
Prediction accuracy of resistant gene list on test samples.

| Predictor | OVERALL(n = 10) | SENS(n = 6) | RES(n = 4) |
|---|---|---|---|
| CCP | 80% | 83% | 75% |
| DLDA | 90% | 100% | 75% |
| 1-NN | 80% | 83% | 75% |
| 3-NN | 90% | 100% | 75% |
| NC | 80% | 83% | 75% |
| SVM | 80% | 83% | 75% |

These studies suggest that the 31 chemoresistant specific molecules can be used to predict chemoresistance in subjects with ovarian cancer with high specificity and sensitivity.

### EXAMPLE 4

### Array Validation

This example provides further support for the use of the specific chemotherapy sensitivity-related molecules provided in Examples 2 and 3 to predict a subject's responsiveness to chemotherapy.

Real-time quantitative RT-PCR (qRT-PCR) was performed to validate the results of the cDNA microarray analysis. A subset of genes was selected from each of the classifier lists.

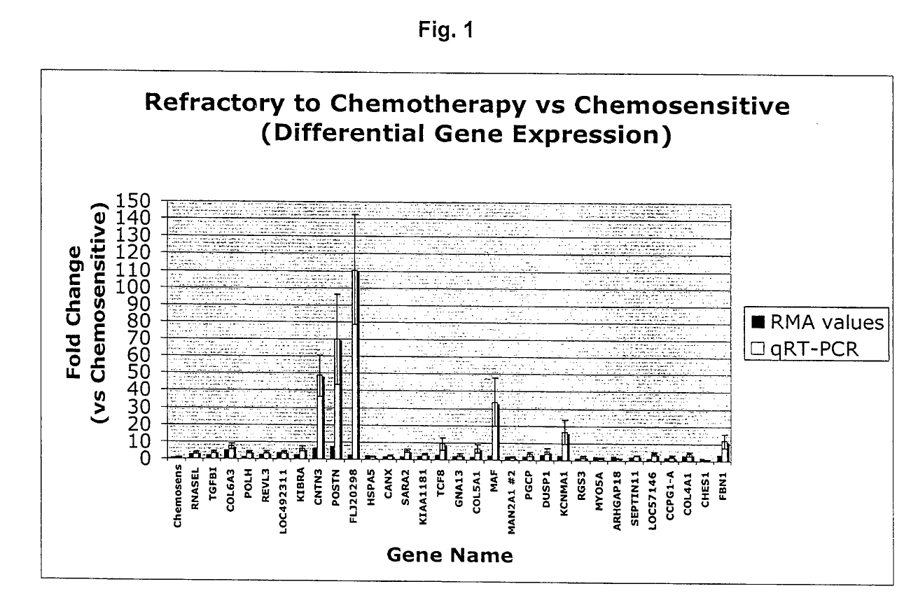

FIG. 1 shows the comparative fold change relative expression levels between the microarray data and real-time qRT-PCR data of selected genes from the refractory gene signature list. Significant correlation was observed between microarray expression data and qRT-PCR expression values. Table 9 shows positive Pearson and Spearman rank correlations for 25/34 (74%) selected refractory genes and 27/34 (79%) selected refractory genes.

TABLE 9
Correlation of microarray expression data with qRT-PCR expression
values: chemosensitive/refractory to chemotherapy tumor samples.

| GENE | Pearsons' r | p-value | Spearmans' r | p-value |
|---|---|---|---|---|
| TGFBI | 0.7631 | <0.0001 | 0.732 | <0.0001 |
| RNASEL | 0.8326 | <0.0001 | 0.8072 | <0.0001 |
| POSTN | 0.9453 | <0.0001 | 0.8957 | <0.0001 |
| MAF | 0.7845 | <0.0001 | 0.4362 | 0.0293 |
| KIBRA | 0.8068 | <0.0001 | 0.7436 | <0.0001 |
| GNA13 | 0.8034 | <0.0001 | 0.4712 | 0.0174 |
| FBN1 | 0.8478 | <0.0001 | 0.7968 | <0.0001 |
| EDIL3 | 0.9359 | <0.0001 | 0.7877 | <0.0001 |
| CTSE | 0.7749 | <0.0001 | 0.502 | 0.0106 |
| COL6A3 | 0.8277 | <0.0001 | 0.7809 | <0.0001 |
| COL5A1 | 0.8604 | <0.0001 | 0.5323 | 0.0062 |
| CNTN3 | 0.7593 | <0.0001 | 0.7745 | <0.0001 |
| LOC492311 | 0.6775 | 0.0002 | 0.6882 | 0.0001 |
| HSPA5 | 0.672 | 0.0002 | 0.6482 | 0.0005 |
| FLJ20298 | 0.6858 | 0.0002 | 0.7599 | <0.0001 |
| TCF8 | 0.642 | 0.0005 | 0.7744 | <0.0001 |
| POLH | 0.6275 | 0.0008 | 0.8311 | <0.0001 |
| DUSP1 | 0.6107 | 0.0012 | 0.4485 | 0.0245 |
| PGCP | 0.5676 | 0.0031 | 0.516 | 0.0083 |
| COL4A1 | 0.5184 | 0.0079 | 0.6355 | 0.0006 |
| RGS3 | 0.4968 | 0.0115 | 0.2535 | 0.2214 |
| CANX | 0.4929 | 0.0123 | 0.5541 | 0.0041 |
| MAN2A1 | 0.488 | 0.0133 | 0.4705 | 0.0176 |
| KCNMA1 | 0.46 | 0.0207 | 0.0446 | 0.8323 |
| REVL3 | 0.4514 | 0.0235 | 0.6528 | 0.0004 |
| KIAA1181 | 0.359 | 0.078 | 0.6168 | 0.001 |
| SARA2 | 0.3562 | 0.0806 | 0.5005 | 0.0108 |
| CDK5R1 | 0.2377 | 0.2634 | 0.5633 | 0.0042 |
| LOC57146 | 0.2019 | 0.3332 | 0.4508 | 0.0237 |
| MYO5A | 0.2505 | 0.2271 | 0.2462 | 0.2356 |
| CCPG1 | 0.1482 | 0.4796 | 0.2369 | 0.2542 |
| CHES1 | −0.0826 | 0.6946 | 0.2616 | 0.2065 |
| ARHGAP18 | −0.0440 | 0.8348 | 0.117 | 0.5775 |
| SEPTIN11 | −0.0109 | 0.959 | 0.2312 | 0.2662 |

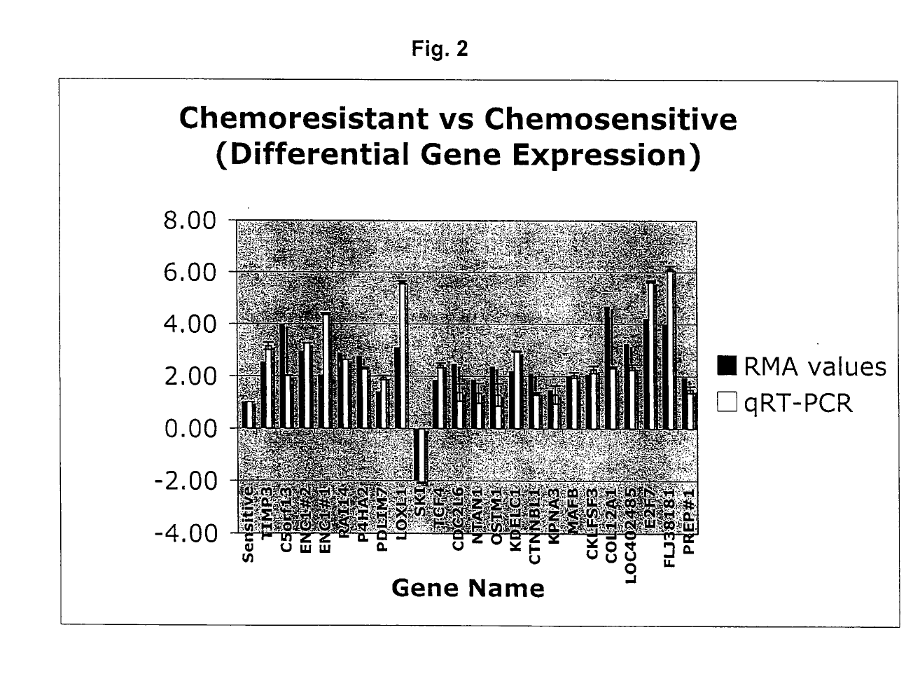

FIG. 2 provides the comparative fold change relative expression levels between the microarray data and real-time qRT-PCR data of selected genes from the resistant gene signature list. Significant correlation was observed between microarray expression data and qRT-PCR expression values. Table 10 shows positive Pearson and Spearman rank correlations for 17/23 (74%) selected chemoresistant genes and 22/23 (96%) selected chemoresistant genes.

TABLE 10
Correlation of microarray expression data with qRT-PCR expression
values: chemosensitive/resistant tumor samples.

| GENE | Pearsons' r | p-value | Spearmans' r | p-value |
|---|---|---|---|---|
| TIMP3 | 0.7617 | <0.0001 | 0.9245 | <0.0001 |
| TCF4 | 0.7902 | <0.0001 | 0.8218 | <0.0001 |
| KDELC1 | 0.7269 | <0.0001 | 0.8759 | <0.0001 |
| E2F7 | 0.7854 | <0.0001 | 0.4349 | 0.0381 |
| LOXL1 | 0.6739 | 0.0004 | 0.8024 | <0.0001 |
| PREP#1 | 0.6694 | 0.0005 | 0.7105 | 0.0001 |
| LOC402485 | 0.668 | 0.0005 | 0.8386 | <0.0001 |
| SKI | 0.6322 | 0.0012 | 0.6273 | 0.0014 |
| MAFB | 0.6297 | 0.0013 | 0.8254 | <0.0001 |
| P4HA2 | 0.6055 | 0.0022 | 0.7527 | <0.0001 |
| CKLFSF3 | 0.5765 | 0.004 | 0.6561 | 0.0007 |
| FLJ38181 | 0.5509 | 0.0064 | 0.8393 | <0.0001 |
| COL12A1 | 0.5413 | 0.0076 | 0.7601 | <0.0001 |
| C5orf13 | 0.5393 | 0.0079 | 0.7679 | <0.0001 |
| ENC1#2 | 0.5244 | 0.0102 | 0.8497 | <0.0001 |
| KPNA3 | 0.5038 | 0.0142 | 0.5425 | 0.0075 |
| RAI14 | 0.4719 | 0.023 | 0.7182 | 0.0001 |
| ENC1#1 | 0.4341 | 0.0385 | 0.6739 | 0.0004 |
| CDC2L6 | 0.351 | 0.1006 | 0.6317 | 0.0012 |
| CTNNBL1 | 0.3438 | 0.1082 | 0.4557 | 0.0289 |
| OSTM1 | 0.2753 | 0.2037 | 0.5078 | 0.0134 |
| PDLIM7 | 0.2703 | 0.2123 | 0.4408 | 0.0353 |
| NTAN1 | 0.2327 | 0.2853 | 0.4242 | 0.0436 |
| PREP#3 | −0.1336 | 0.5535 | 0.08077 | 0.7209 |

These studies provide further support for the use of the specific chemotherapy sensitivity-related molecules provided in Examples 2 and 3 to predict a subject's responsiveness to chemotherapy.

### EXAMPLE 5

### Effect of POLH and/or REV3L siRNAs on Cisplatin Sensitivity

This example describes the effect of pretreating ovarian tumor cells with POLH and/or REV3L siRNAs prior to chemotherapy to increase sensitivity to cisplatin. Although specific siRNAs are described, one skilled in the art will appreciate that others can be used.

As described above in Example 1, A2780CP20 ovarian cancer cell lines were transfected with siPOLH-2 or siPOLH-5, treated with cisplatin starting 48 hours later, and assayed with MTS 48 hours thereafter. Viable cell number data was acquired by reading the fluorescence emissions at 490 nm. Using GraphPad Prism 4.02, cisplatin drug concentrations were log-transformed and nonlinear regression performed on the A490 data using the sigmoidal dose response model with variable slope to generate the IC₅₀ curves. IC₅₀ values and 95% confidence intervals were determined from the logistic fits.

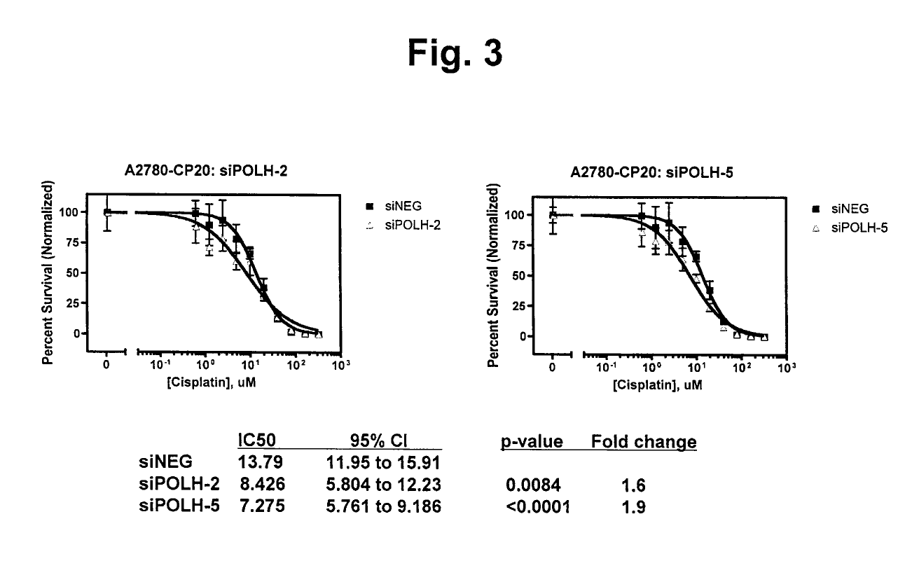

As illustrated in FIG. 3, siPOLH-2 or siPOLH-5 pretreatment of A2780CP20 ovarian cancer cells significantly increased cell sensitivity to cisplatin when compared to cells treated with siNEG (p=0.0084 for siPOLH-2 and <0.0001 for siPOLH-5). For example, the IC₅₀ for cisplatin following siPOLH-2 pretreatment was 8.426 μM and that following siPOLH-5 pretreatment was 7.275 μM. Further, a 1.6 fold change in cisplatin sensitivity was detected for siPOLH-2 pretreatment and a 1.9 fold change in such sensitivity for siPOLH-5 pretreatment.

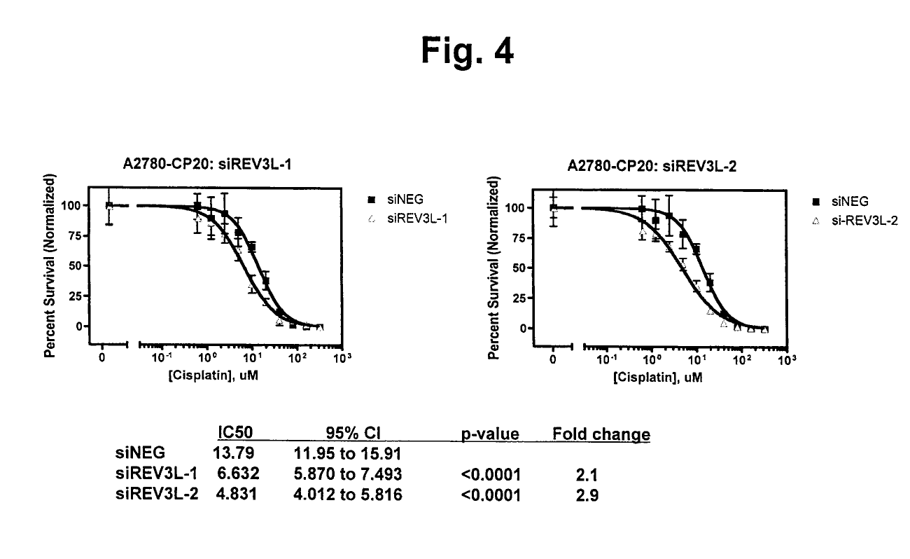

FIG. 4 demonstrates the effect of RNAi against REV3L on cisplatin resistance. A2780CP20 ovarian cancer cell lines were transfected with siREV3L-1 or siREV3L-2, treated with cisplatin starting 48 hours later, and assayed with MTS 48 hours thereafter (as described above, including Example 1). Viable cell number data was acquired by reading the fluorescence emissions at 490 nm. Using GraphPad Prism 4.02, cisplatin drug concentrations were log-transformed and nonlinear regression performed on the A490 data using the sigmoidal dose response model with variable slope to generate the IC₅₀ curves. IC₅₀ values and 95% confidence intervals were determined from the logistic fits. As illustrated in FIG. 4, siREV3L-1 or siREV3L-2 pretreatment of A2780CP20 ovarian cancer cells significantly increased cell sensitivity to cisplatin when compared to cells treated with siNEG (p<0.0001 for both siREV3L-1 or siREV3L-2). For example, the IC₅₀ for cisplatin following siREV3L-1 pretreatment was 6.632 μM and that following siPOLH-5 pretreatment was 4.831 μM. Further, a 2.1 fold change in cisplatin sensitivity was detected for siREV3L-1 pretreatment and a 2.9 fold change in such sensitivity for siREV3L-2 pretreatment.

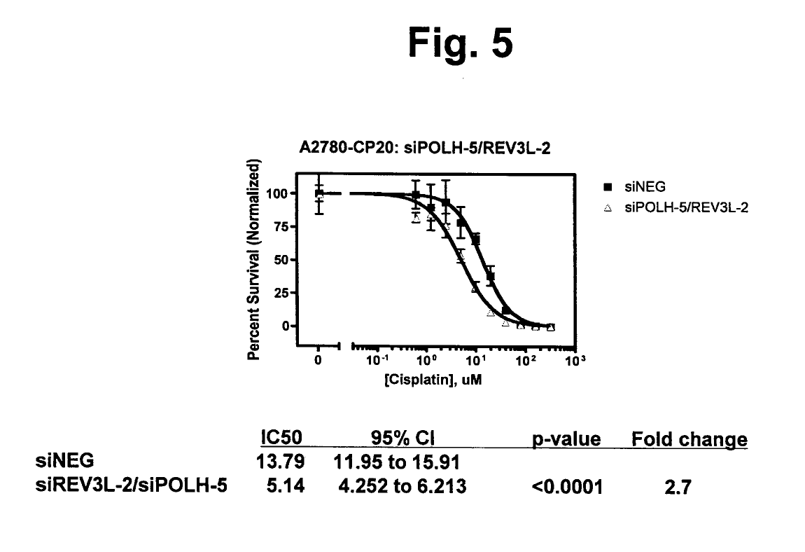

FIG. 5 illustrates the effect of pretreatment with RNAi against POLH and REV3L on cisplatin resistance. A2780CP20 ovarian cancer cell lines were cotransfected with siPOLH-5 and siREV3L-2, treated with cisplatin starting 48 hours later, and assayed with MTS 48 hours thereafter. Viable cell number data was acquired by reading the fluorescence emissions at 490 nm. Using GraphPad Prism 4.02, cisplatin drug concentrations were log-transformed and nonlinear regression performed on the A490 data using the sigmoidal dose response model with variable slope to generate the IC₅₀ curves. IC₅₀ values and 95% confidence intervals were determined from the logistic fits.

As illustrated in FIG. 5, siREV3L-2 and siPOLH-5 pretreatment of A2780CP20 ovarian cancer cells significantly increased cell sensitivity to cisplatin when compared to cells treated with siNEG (p<0.0001). For example, the IC₅₀ for cisplatin following pretreatment was 5.14 μM. Further, a 2.7 fold change in cisplatin sensitivity was detected with siREV3L-2/siPLH-5 pretreatment.

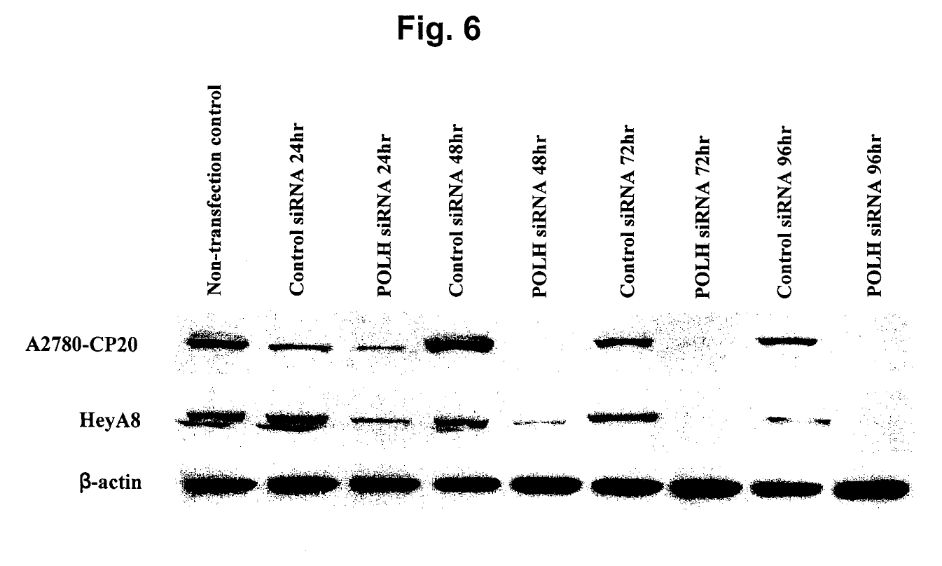

FIG. 6 illustrates the ability of POLH siRNA to inhibit POLH RNA expression following 24 hours, 48 hours, 72 hours or 96 hours treatment with siPOLH-5 RNA. Cell lysates were collected and examined by Western blot analysis for POLH. After treating cells with 5 ul of 1 ug/ul POLH-siRNA, lysates were collected at 24, 48, 72 and 96 hours and then analyzed for down-regulation of POLH.

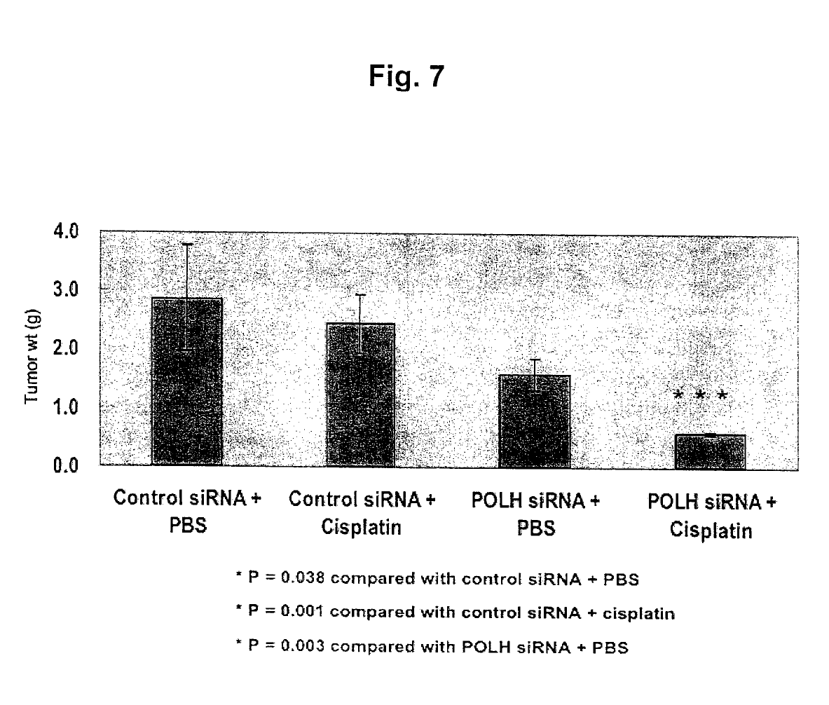

FIG. 7 is a bar graph illustrating the ability of POLH siRNA and cisplatin therapy to significantly reduce tumor weight. As illustrated in FIG. 7, tumor weight was significantly reduced by treating A2780CP20 with POLH siRNA (150 ug/kg) prior to treatment with cisplatin (160 ug per week). Nude mice were injected i.p. with A2780-CP20 and randomly allocated to one of the following groups, with therapy beginning 1 week after tumor cell injection: control siRNA in a neutral liposome 1,2-dioleoyl-sn-glycero-3-phosphatidylcholine (DOPC)+PBS, control siRNA in DOPC+cisplatin, POLH siRNA in DOPC+PBS, and POLH siRNA in DOPC+cisplatin. The animals were sacrificed when control mice became moribund (4-5 weeks after starting therapy) and necropsy was done. (mean±SE)

These studies demonstrate that ovarian cancer cell sensitivity to cisplatin can be increased by pretreating cells with POLH and/or REV3L siRNAs. It is expected that similar results can be derived with siRNAs for any of the genes in Tables 1 or 5 with a positive t-value.

### EXAMPLE 6

### Predicting Chemotherapy Sensitivity

This example describes methods that can be used to predict chemotherapy sensitivity in a subject with cancer, such as ovarian cancer.

According to the teachings herein, a subject's responsiveness to chemotherapy can be predicted by detecting differential expression of at least six chemotherapy sensitivity-related molecules in a sample obtained from the subject with ovarian cancer, such as papillary serous ovarian carcinoma. In an example, the at least six chemotherapy sensitivity-related molecules are represented by any combination of the molecules listed in any of Tables 1 and 5. The presence of differential expression of at least six chemotherapy sensitivity-related molecules indicates that the ovarian cancer has a decreased sensitivity to chemotherapy treatment. The expression product can be RNA or protein. An RNA expression product can be detected by a microarray or PCR by methods described above (see, for example, Example 1). A protein expression product can be detected by standard Western blot or immunoassay techniques that are known to one of skill in the art. However, the disclosure is not limited to particular methods of detection.

### EXAMPLE 7

### Identification of Chemotherapy Sensitivity-Related Molecule Inhibitors to Alter Chemoresponsiveness

This example describes methods that can be used to identify chemotherapy sensitivity-related molecule inhibitors that can be used to target specific genes whose increased expression is associated with the chemoresistant/chemorefractory phenotype, such as COL5A1, COL1A1, DUSP1, REV3L, RNASEL, and POLH with positive t-values in Table 1.

Based upon the teaching disclosed herein, iSynthetic siRNA molecules are generated against selected target genes, such as any of the chemorefractory or chemoresistant genes identified in Examples 2 through 4 whose increased expression is associated with chemorefraction or chemoresistance. Knockdown efficiency of the siRNA molecules can be assessed by comparing target siRNA knockdown to the control siRNA molecules (siNEG). In an example, a significant knockdown efficiency is at least 20%. As provided in Example 1, select ovarian cancer cell lines are transfected with target gene siRNA or control siNEG molecules, and the IC₅₀ values to chemotherapeutic reagents such as cisplatin or taxol are determined. The IC₅₀ values are compared (between target gene siRNA and siNEG molecules) to determine whether the gene targeted for knockdown affects the sensitivity of the ovarian cancer cell line to the chemotherapeutic reagent (e.g., cisplatin or taxol).

In additional examples, two or more siRNAs (that target two or more genes) are transfected into select ovarian cancer cells and the IC₅₀ values to chemotherapeutic reagents are determined. The IC₅₀ values are compared (between target gene siRNA individually and in combination) to determine whether the knockdown effect on chemotherapy drug sensitivity is cumulative or additive.

siRNAs that are determined to have a knockdown efficiency of at least 20% are chosen for further study. For example, the effect of these siRNA(s) on the ability of an animal model of chemoresistant or chemorefractory ovarian cancer (such as, orthotopic models using resistant cell lines) to respond to chemotherapy is determined.

### EXAMPLE 8

### Inhibition of Chemoresistance

This example describes methods that can be used to significantly reduce chemorefraction/chemoresistance in a subject with ovarian cancer, such as papillary serous ovarian carcinoma.

Based upon the teaching disclosed herein, chemorefraction/chemoresistance can be reduced or inhibited by administering a therapeutically effective amount of a composition, wherein the composition comprises a specific binding agent that preferentially binds to one or more chemotherapy sensitivity-related molecules provided in Tables 1 and 5 that are up-regulated in chemorefractory or chemoresistant ovarian tumors, thereby reducing or inhibiting chemorefraction/chemoresistance in the subject.

In an example, a subject who has been diagnosed with ovarian cancer is identified and then determined if chemoresistant or chemorefractory by any of the methods disclosed herein. Following subject selection, a therapeutic effective dose of the composition including the specific binding agent is administered to the subject. For example, a therapeutic effective dose of a specific binding agent to one or more of the disclosed chemotherapy sensitivity-related molecules is administered to the subject to inhibit chemorefraction/chemoresistance. In an example, the specific binding agent is a siRNA. In a further example, the specific binding agent is an antibody. The amount of the composition administered to prevent, reduce, inhibit, and/or treat chemorefraction/chemoresistance or a condition associated with it depends on the subject being treated, the severity of the disorder, and the manner of administration of the therapeutic composition. Ideally, a therapeutically effective amount of an agent is the amount sufficient to prevent, reduce, and/or inhibit, and/or treat the condition (e.g., chemorefraction/chemoresistance) in a subject without causing a substantial cytotoxic effect in the subject.

In one specific example, siRNAs are administered at according to the teachings of Soutschek et al. (Nature Vol. 432: 173-178, 2004) or Karpilow et al. (Pharma Genomics 32-40, 2004) both of which are herein incorporated by reference in their entireties. In one example, siRNAs are incorporated into neutral liposomes, such as DOPC, and injected intraperitoneal or intravenously. For example, a siRNA is administered at 150 μg/kg twice weekly for 2 to 3 weeks. In another specific example, naked antibodies are administered at 5 mg per kg every two weeks or 10 mg per kg every two weeks depending upon the chemorefraction/chemoresistance. In an example, the antibodies are administered continuously. In another example, antibodies or antibody fragments conjugated to cytotoxic agents (immunotoxins) are administered at 50 μg per kg given twice a week for 2 to 3 weeks. In other examples, the subject is administered the therapeutic composition that a binding agent specific for one or more of the disclosed chemotherapy sensitivity-related molecules daily for a period of at least 30 days, such as at least 2 months, at least 4 months, at least 6 months, at least 12 months, at least 24 months, or at least 36 months.

Subjects will monitored by methods known to those skilled in the art to determine ovarian tumor responsiveness to the siRNA or antibody treatment. The subject will be monitored by non invasive techniques such as CT or MRI imaging to assess tumor response. It is contemplated that additional agents can be administered, such as antineoplastic agents in combination with or following treatment with the siRNA or antibodies.

While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims. We therefore claim as our invention all that comes within the scope and spirit of these claims.

1. A method of determining if a subject with ovarian cancer is sensitive to treatment with a chemotherapeutic agent, comprising:
detecting expression of at least six chemotherapy sensitivity-related molecules in a sample obtained from the subject, wherein the at least six chemotherapy sensitivity-related molecules are represented by any combination of molecules listed in any of Tables 1 and 5, and wherein the presence of differential expression of the at least six chemotherapy sensitivity-related molecules as compared to a reference value indicates that the ovarian cancer has a decreased sensitivity to the chemotherapeutic agent.

2. The method of claim 1, wherein the method comprises determining if the ovarian cancer is refractory and wherein the at least six chemotherapy sensitivity-related molecules are represented by any combination of molecules listed in Table 1.

3. The method of claim 2, wherein the at least six chemotherapy sensitivity-related molecules consist of COL5A1, COL1A1, DUSP1, REV3L, RNASEL, and POLH.

4. The method of claim 2, wherein the method has a specificity of at least 83% and a sensitivity of at least 71%.

5. The method of claim 2, wherein the at least six chemotherapy sensitivity-related molecules consist of eighty of the chemotherapy sensitivity-related molecules listed in Table 1.

6. The method of claim 2, wherein the method comprises detecting differential expression of one-hundred and five chemotherapy sensitivity-related molecules listed in Table 1.

7. The method of claim 1, wherein the method comprises determining if the ovarian cancer is resistant and wherein the at least six chemotherapy sensitivity-related molecules are represented by any combination of molecules listed in Table 5.

8. The method of claim 7, wherein the method has a specificity of at least 83% and a sensitivity of at least 77%.

9. The method of claim 1, wherein the at least six chemotherapy sensitivity-related molecules are RNA.

10. The method of claim 1, wherein the chemotherapy sensitivity-related molecules are protein.

11. The method of claim 1, wherein the subject is a human.

12. The method of claim 1, wherein the ovarian cancer is papillary serous ovarian cancer.

13. The method of claim 1, wherein the chemotherapeutic agent comprises a platinum-based chemotherapeutic agent.

14. The method of claim 13, wherein the platinum-based chemotherapeutic agent comprises cisplatin.

15. The method of claim 14, wherein the chemotherapeutic agent further comprises paclitaxel.

16. The method of claim 1, wherein detecting whether there is differential expression of at six chemotherapy sensitivity-related molecules comprises determining whether a gene expression profile from the subject indicates chemoresponsiveness.

17. The method of claim 16, wherein the gene expression profile is generated using an array of molecules comprising a chemoresponsiveness expression profile.

18. The method of claim 2, further comprising administering to the subject a therapeutically effective amount of a treatment to increase the ovarian cancer sensitivity to the chemotherapeutic agent if the presence of differential expression indicates that the ovarian cancer is refractory to the chemotherapeutic agent.

19. The method of claim 18, wherein the treatment comprises administration of a therapeutically effective amount of a composition, comprising one or more specific binding agents that preferentially bind to one or more chemotherapy sensitivity-related molecules listed in Table 1, thereby increasing the ovarian cancer's sensitivity to the chemotherapeutic agent.

20. The method of claim 19, wherein the one or more specific binding agents preferentially bind to RNASEL, POLH, COL5A1, DUSP1, REV3L, and COL1A1.

21. The method of claim 19, wherein the one or more specific binding agents are inhibitors of one or more of the chemotherapy-sensitivity related molecules.

22. The method of claim 21, wherein the inhibitors are one or more siRNAs comprising at least 95% sequence identity to any one of SEQ ID NOs: 2, 3, 5, 6, 8, 9, 11, or 12.

23. The method of claim 7, further comprising administering to the subject a therapeutically effective amount of a treatment to increase ovarian cancer sensitivity to the chemotherapeutic agent if the presence of differential expression indicates that the ovarian cancer is resistant to the chemotherapeutic agent.

24. The method of claim 23, wherein the treatment comprises administration of a therapeutically effective amount of a composition, comprising one or more specific binding agents that preferentially binds to one or more chemotherapy sensitivity-related molecules listed in Table 5, thereby increasing the ovarian cancer's sensitivity to the chemotherapeutic agent.

25. The method of claim 24, wherein the specific binding agents are inhibitors of one or more of the chemotherapy-sensitivity related molecules.

26. The method of claim 25, wherein the inhibitors are siRNA.

27. The method of claim 1, wherein detecting expression of at least six chemotherapy sensitivity-related molecules in a sample obtained from the subject is performed by using a reverse-transcription-polymerase chain reaction (RT-PCR).

28. The method of claim 27, wherein the RT-PCR comprises quantitative RT-PCR.

29. A method of evaluating chemoresponsiveness in a subject with ovarian cancer, comprising:
applying isolated nucleic acid molecules obtained from a biological sample including ovarian cancer cells to an array, wherein the array comprises oligonucleotides complementary to all chemotherapy sensitivity-related genes listed in Table 1 and/or Table 5;
incubating the isolated nucleic acid molecules with the array for a time sufficient to allow hybridization between the isolated nucleic acid molecules and oligonucleotide probes, thereby forming isolated nucleic acid molecule:oligonucleotide complexes;
analyzing the isolated nucleic acid molecule:oligonucleotide complexes to determine if expression of the isolated nucleic acid molecules is altered, wherein the presence of differential expression in at least six of the genes indicates that the ovarian cancer cells have a decreased sensitivity to chemotherapy treatment.

30. The method of claim 29, wherein the array comprises oligonucleotides complementary to all chemotherapy sensitivity-related genes listed in Table 5, and wherein the presence of differential expression in at least six of the chemotherapy sensitivity-related molecules genes indicates that the ovarian cancer cells are resistant to chemotherapy treatment.

31. The method of claim 29, wherein the array comprises oligonucleotides complementary to all chemotherapy sensitivity-related genes listed in Table 1, and wherein the presence of differential expression of at least six of the chemotherapy sensitivity-related molecules genes indicates that the ovarian cancer cells are refractory to chemotherapy treatment.

32. The method of claim 29, wherein analyzing the isolated nucleic acid molecule:oligonucleotide complexes to determine if expression of the isolated nucleic acid molecules is altered is performed by using a reverse-transcription-polymerase chain reaction (RT-PCR).

33. The method of claim 32, wherein the RT-PCR comprises quantitative RT-PCR.

34.-40. (canceled)

41. A kit, consisting essentially of agents specific for chemotherapy sensitivity-related molecules listed in Tables 1, 5 or a combination thereof.

42. The kit of claim 41, consisting of agents specific for chemotherapy sensitivity related molecules listed in Table 1 or Table 5 and one to ten controls.

43. (canceled)

44. (canceled)


SEQUENCE LISTING (15 sequences)

SEQ ID NO: 1 - DNA, 39 bp, Artificial Sequence
  synthetic oligonucleotide; oligo-dt24-T7
ggccagtgaattgtaatacgactcactatagggaggcgg

SEQ ID NO: 2 - DNA, 21 bp, artificial sequence
  small inhibitory RNA; sense sequence of siREV3l.1
ggauguagucaaacugcaatt

SEQ ID NO: 3 - DNA, 21 bp, artificial sequence
  small inhibitory RNA; siREV3L..1 antisense sequence
uugcaguuugacuacauccag

SEQ ID NO: 4 - DNA, 21 bp, homo sapien
cgggatgtagtcaaactgcaa

SEQ ID NO: 5 - DNA, 21 bp, Artificial sequence
  small inhibitory RNA; siREV3L.2 sense sequence
cacuggaauuaaugcacaatt

SEQ ID NO: 6 - DNA, 21 bp, artificial sequence
  small inhibitory RNA; siREV3L.2 antisense sequence
uugugcauuaauuccagugtg

SEQ ID NO: 7 - DNA, 21 bp, homo sapien
cccactggaattaatgcacaa

SEQ ID NO: 8 - DNA, 21 bp, artificial sequence
  small inhibitory RNA; siRNA for POLH-2 sense sequence
ccauuuaggugcugaguuatt

SEQ ID NO: 9 - DNA, 21 bp, artificial sequence
  small inhibitory RNA; POLH-2 antisense sequence
uaacucagcaccuaaauggag

SEQ ID NO: 10 - DNA, 21 bp, homo sapien
atccatttaggtgctgagtta

SEQ ID NO: 11 - DNA, 21 bp, Artificial sequence
  small inhibitory RNA; siPOLH-5 sense sequence
gguugugagcauucguguatt

SEQ ID NO: 12 - DNA, 21 bp, Artificial sequence
  small inhibitory RNA; siPOLH-5 antisense sequence
uacacgaaugcucacaacctg

SEQ ID NO: 13 - DNA, 21 bp, homo sapien
ctggttgtgagcattcgtgta

SEQ ID NO: 14 - DNA, 21 bp, artificial sequence
  small inhibitory RNA; siNegative control sense sequence
uucuccgaacgugucacgutt

SEQ ID NO: 15 - DNA, 21 bp, artificial sequence
  small inhibitory RNA; siNegative control antisense sequence
acgugacacguucggagaatt